(19) United States
(12) Patent Application Publication
(10) Pub. No.: US 2008/0267981 A1
(43) Pub. Date: Oct. 30, 2008

(54) Compositions and Methods for Delivery of Antitumor Agents

(75) Inventors: Kim D. Janda, La Jolla, CA (US); Peter Wirsching, Del Mar, CA (US); Dale L. Boger, La Jolla, CA (US)

Correspondence Address: THE SCRIPPS RESEARCH INSTITUTE, OFFICE OF PATENT COUNSEL, TPC-8, 10550 NORTH TORREY PINES ROAD, LA JOLLA, CA, 92037, US

(73) Assignee: The Scripps Research Institute, La Jolia, CA (US)

(21) Appl. No.: 11/630,101

(22) PCT Filed: Jun. 29, 2005
(86) PCT No.: PCT/EP05/07007

§ 371 Date: Nov. 30, 2007

Related U.S. Application Data
(60) Provisional application No. 60/584,226, filed on Jun. 30, 2004.

(51) Int. Cl. A61K 39/395; C07K 16/00; C07K 16/46; C07D 209/04; C07D 209/90; A61P 35/00; C07D 401/00

(52) U.S. Cl. 424/181.1; 424/178.1; 530/391.7; 548/492; 548/433; 548/427; 548/420; 548/421

(57) ABSTRACT

Methods for treating a neoplastic disease with an antibody-cytotoxin conjugate molecule, methods of synthesizing an antibody-cytotoxin conjugate molecule are provided. Compounds that are useful as antibody-cytotoxin conjugate molecule or useful in the synthesis of these molecules are also provided.

This application is based on U.S. Provisional Application No. 60/584,226, filed Jun. 30, 2004, the contents of which are hereby incorporated by reference.

This work was supported by the Skaggs Institute for Chemical Biology, the NIH (grants NAID-AI47127, NCI-CA41986 and RO1-HL63651), the California Cancer Research Program (grant 00-00757V-20012), the California Breast Cancer Research Program (grant 4JB-001) and the Louis R. Jabinson Fellowship (Louis R. Jabinson Investigatorship Fund for Graduate Education). The government may have certain rights in this invention.

### FIELD

The invention generally relates to methods for treating a neoplastic disease with an antibody-cytotoxin conjugate molecule, methods of synthesizing an antibody-cytotoxin conjugate molecule, and compounds that are useful as antibody-cytotoxin conjugate molecules or useful in the synthesis of these molecules.

### BACKGROUND

Targeted treatment of tumors has advanced considerably in the last two decades, primarily due to the establishment of monoclonal antibody (mAb) technology. Kohler et al., Nature, 256: 495-497, 1975. An early fundamental application was the development of radiolabeled mAbs, some of which have attained clinical use for imaging and cancer therapy. Kousparou et al., J Int Soc Tumor Target, 1: 55-69, 2000; Buchsbaum et al. Antibody Immunoconjugate Radiopharm, 4: 245-272, 1991. Significantly, mAb-drug conjugates are another potential class of anticancer agents that have been extensively investigated. Safavy et al., In Drug Targeting in Cancer Therapy, M. Page, ed., 257-275, 2002; Stan et al., Cancer Res, 59:115-121, 1999; Florent et al., J Med Chem, 41: 3572-3581, 1998. However, although isolated examples of success have been reported, considerable advances are necessary in order to address the complex issue of cancer treatment.

A central goal has been the search for human mAbs or peptides that can be specifically internalized by tumor cells upon binding to overexpressed cell-surface receptors or ligands. Nielsen et al, Pharm. Sci. Technol. Today, 3: 282-291, 2000; Trail et al., Cancer Immunol Immunother, 52: 328-337, 2003; Gao et al, J Immunol Methods, 274: 185-197, 2003; Gao et al., Bioorg Med Chem, 10: 4057-4065, 2002. This line of research presents opportunities to use protein vectors to deliver drug payloads that can increase the efficacy and lessen side-effects of cancer chemotherapy. One demonstration of the clinical potential for such a strategy invoked the cell internalizing anti-CD33 antibody P67.6 conjugated to calicheamicin for use against acute myeloid leukemia that has resulted in the FDA approved drug Mylotarg™. Hamann et al., Bioconjug Chem, 13: 40-46, 2002.

Integrin (α₃β₁, also known as the VLA-3 membrane receptor, is expressed by both fetal and adult tissues mediating adhesive, migratory and invasive cell interactions with the extracellular matrix. Elices et al., J Cell Biol, 112:169-181, 1991. Elevated expression of α₃β₁ has been observed in several types of metastatic cancer types and has been associated with increased migration and invasion. Notably, expression of this integrin is upregulated in malignant melanoma and correlates well with the degree of migration and dermal invasiveness. Melchiori et al., Exp Cell Res, 219: 233-242, 1995; Laidler et al., Acta Biochim Pol 47: 1159-1170, 2000; Elshaw et al., Br J Opthalmol, 85: 732-738, 2001; Yoshinaga et al., Melanoma Res, 3:435-441, 1993. The α₃β₁ integrin is also expressed by invasive clones of human PC-3 prostate carcinoma cells, but not by the noninvasive parent cell population. Dedhar et al., Clin Exp Metastasis, 11: 391-400, 1993; Romanov et al., Prostate, 39: 108-118, 1999. Similarly, the invasive properties of different squamous cell cancers have been correlated to overexpression of several integrins including α₃β₁. Dyce et al., Laryngoscope, 112: 2025-2032, 2002; Ghosh et al., Cancer, 95: 2524-2533, 2002. It has also been shown that functional inhibition of α₃β₁ in malignant glioma cells can block their invasive ability. Fukushima et al., Int J Cancer, 76: 63-72, 1998. The α₃β₁ is also associated with mammary carcinoma cell metastasis, invasion, and collagen degradation activity. Morini et al., Int J Cancer, 87: 336-342, 2000. Finally, expression of (α₃β₁ in murine hepatocellular carcinoma (HCC) has been associated with the occurrence of intrahepatic metastasis, which is considered to be a major modality in recurrence. Tsuchiya et al., Int J Oncol, 20: 319-324, 2002. Given the often distinct levels of expression between malignant cancer cells and normal cells, α₃β₁ can be considered a viable target for a specific antibody-based antineoplastic treatment designed to kill cancer cells and control metastatic dissemination.

Selective control of metastasis by targeting α₃β₁ has been shown to be successful in the treatment of intrahepatic metastasis of hepatocellular carcinoma (HCC) using an RGD (Arginine-Glycine-Aspartate) pseudopeptide. Tsuchiya et al., Int J Oncol, 20: 319-324, 2002. Also, squamous cell carcinoma of the head and neck has been treated by selective gene delivery via an α₃β₁ integrin-targeted adenoviral vector. Kasono et al., Clin Cancer Res, 5: 2571-2579, 1999. Several murine mAbs are known to target either the α₃ or β₁ subunits of α₃β₁, however, none are known to be internalized by tumor cells nor have they ever been used as anticancer therapeutics. Morimoto et al., J Immunol, 134: 3762-3769, 1985; Wayner et al., J Cell Biol, 105: 1873-1884, 1987; Bartolazzi et al., Anticancer Res, 13: 1-11, 1993. Significantly, the typical murine origin of most mAbs is a detriment for human clinical application. Tjandra et al., Immunol Cell Biol, 68: 367-376, 1990; Schroff et al., Cancer Res, 45: 879-885, 1985; Goldman-Leikin et al., Exp Hematol, 16: 861-864, 1988; Heriyn et al., J Immunol Methods, 85: 27-38, 1985. In addition, another barrier can be the effective use of a mAb as whole immunoglobulin G (IgG), generally attributed to the high molecular weight, which hinders efficient penetration of solid tumors. For instance, studies have indicated that less than 1% of an infused radiolabeled IgG can reach its target tumor mass. Jain, Cancer Res, 50: 814s-819s, 1990; Pimm et al., In Monoclonal Antibodies for Cancer Detection and Therapy, V. S. Byers, ed., 97-128, 1985. One method to circumvent this problem is the use of a mAb in the scFv format. Compared to whole IgG, and the fragments Fab and F(ab′)2, scFvs have been shown to permeate more rapidly and deeper into tumors in addition to demonstrating very rapid plasma and body clearance (<30 min). Chester et al., Trends Biotechnol, 13: 294-300, 1995; Hand et al., Cancer, 73: 1105-1113, 1994; Yokota et al., Cancer Res, 52: 3402-3408, 1992; Milenic et al., Cancer Res, 51: 6363-6371, 1991; Colcher et al., J Natl Cancer Inst, 82: 1191-1197, 1990. Therefore, in many cases, a preferred therapeutic strategy can be the use of a human scFv conjugated with an anticancer agent.

CC-1065 and duocarmycin are two antitumor antibiotics possessing sequence selective DNA alkylation properties. Chidester et al., J Am Chem Soc, 103: 7629-7635, 1981; Takahashi et al., J Antibiot (Tokyo), 41: 1915-1917, 1988; Ichimura et al., J Antibiot (Tokyo), 43: 1037-1038, 1990; Yasuzawa et al., Chem Pharm Bull (Tokyo), 43: 378-391, 1995; Boger et al., Angew Chem, Int Ed Engl, 35: 1439-1474, 1996. The development of these anticancer molecules for single-agent therapies has not been pursued because of delayed toxicities that limit the therapeutic dose range for treatment. For instance, despite its high potency and broad spectrum of antitumor activity, CC-1065 is problematic because it has been shown to cause delayed death in experimental animals. Chidester et al., J Am Chem Soc, 103: 7629-7635, 1981. However, these drugs can be well suited for antibody-targeted chemotherapy, where restricted antigen expression makes the potency of the cytotoxic agent crucial and targeting can avert some toxic effects. Liu et al., Exp Opin Invest Drugs, 6: 169-172, 1997; Chari et al., Cancer Res, 55: 4079-4084, 1995. Great efforts have been made to specifically target the high cytotoxicity of these compounds to the tumor mass sparing normal healthy cells. Investigations have included TAP (tumor-activated prodrug) and ADEPT (antibody-directed enzyme prodrug therapy) approaches. Zhao et al., Abstr Pap Am Chem Soc, 224: 147-MEDI Part 142, 2002; Suzawa et al., Bioorg Med Chem, 8: 2175-2184, 2000; Wang et al., BMC Chem Biol, 1: 4, 2001; Tietze et al., Chembiochem, 2: 758-765, 2001; Tietze et al., Bioorg Med Chem 9: 1929-1939, 2001. Both methods are intended to reduce the cytotoxicity of CC-1065 or duocarmycin analogs by conjugating these molecules to substrates of enzymes at the tumor site. In the first study, the targeted enzyme was naturally present in the tumor environment, while in the second study the enzyme was brought to the tumor site upon conjugation to a tumor-specific antibody. Despite their elegance, the main drawbacks of these approaches are the residual cytotoxicity of the prodrugs and the release of the free drug outside the tumor cell. To date, no attempts to deliver duocarmycin analogs specifically into tumor cells by conjugating this drug to antibody fragments have been reported. Duocarmycin, J. Antibiotics, 43:1037, 1990.

Therefore, in spite of the advances in the art, there continues to be a need for the development of improved therapeutic agents, for example for the treatment of neoplastic disease, e.g., cancer and tumors in mammals and humans, in particular. More specifically, the therapeutic agent can be a cytotoxin and related prodrug which can be conjugated to an antibody, that exhibit high specificity of action, reduced toxicity, and improved stability in blood relative to known compounds of similar structure.

### SUMMARY

The invention is generally related to methods for treating a neoplastic disease with an antibody-cytotoxin conjugate molecule, methods of synthesizing an antibody-cytotoxin conjugate molecule, and compounds that are useful as antibody-cytotoxin conjugate molecule or useful in the synthesis of these molecules. Benefits of the present invention can be obtained using antibody-drug conjugates to deliver chemotherapeutic agents more selectively to tumor cells, typically via recognition of a cell-surface epitope. The present invention provides a viable antibody-based therapeutic approach to acquire human antibodies that target, and perhaps internalize, receptors or ligands upregulated on tumor cells as compared to normal cells. One such receptor is integrin α₃β₁ that is overexpressed on some malignant cancer cells. A human single-chain Fv antibody (scFv), denoted Pan 10, specific for integrin ocsPi that is internalized by human pancreatic cancer cells has been identified. The methods of the present invention utilize antibodies to direct potent cytotoxic drugs directly to tumors in a highly selective way, thus reducing indiscriminate cell destruction. These methods will potentially enhance the efficacy of, and also reduce the side effects frequently associated with, chemotherapeutic agents.

The present invention provides chemical introduction of reactive thiol groups onto Pan 10, the specific conjugation of the modified scFv to maleimide-derivatized analogs of the potent cytotoxic agent duocarmycin S A, and the properties of the resultant conjugates. The findings provide evidence that Pan 10-drug conjugates maintain the internalizing capacity of the parent scFv and exhibit cytotoxic activity in vitro at nanomolar concentrations. Pan 10-drug conjugates can be promising candidates for targeted chemotherapy of malignant diseases including melanoma, prostate carcinoma, glioma and other neoplasias involving overexpression of integrin

In one embodiment, a method for treating a neoplastic disease in a mammal comprises providing an antibody-cytotoxin conjugate with an acid-stable covalent linkage between an antibody and a cytotoxin, administering the antibody-cytotoxin conjugate to the mammal, and internalizing the antibody-cytotoxin conjugate within a cell of the mammal to treat the neoplastic disease within the cell of the mammal.

In a detailed embodiment, the cytotoxin is an antitumor antibiotic, duocarmycin, duocarmycin A, duocarmycin SA, or an analog thereof. In a detailed embodiment, the acid-stable linkage is an amide linkage. In a further detailed embodiment, the amide linkage is an N-substituted amide linkage. In a detailed embodiment, the antibody specifically binds to an activated integrin receptor. In a further detailed embodiment, the activated integrin receptor is differentially produced on a cell in a metastatic state as compared to a similar, non-metastatic cell. In a further detailed embodiment, the activated integrin receptor is an α₃β₁ integrin receptor or an α₃β₁ integrin receptor. In a further detailed embodiment, the antibody is a single chain Fv antibody.

In a further embodiment, the neoplastic disease is selected from solid tumor, hematological malignancy, leukemia, colorectal cancer, benign or malignant breast cancer, uterine cancer, uterine leiomyomas, ovarian cancer, endometrial cancer, polycystic ovary syndrome, endometrial polyps, squamous cell carcinoma, squamous cell carcinoma of the head and neck, hepatocellular carcinoma, intrahepatic metastasis of hepatocellular carcinoma, prostate cancer, prostatic hypertrophy, pituitary cancer, adenomyosis, adenocarcinoma, pancreatic adenocarcinoma, meningioma, melanoma, bone cancer, multiple myeloma, CNS cancer, glioma, or astroblastoma.

In another embodiment, a method for treating a neoplastic disease in a mammal comprises providing an antibody-cytotoxin conjugate with an acid-labile covalent linkage between an antibody and a cytotoxin, administering to the mammal the antibody-cytotoxin conjugate, and internalizing the antibody-cytotoxin conjugate within a cell of the mammal to treat the neoplastic disease within the cell of the mammal. In a detailed embodiment, the cytotoxin is an antitumor antibiotic, duocarmycin, duocarmycin A, duocarmycin SA, or an analog thereof. In a detailed embodiment, the acid-labile covalent linkage is a hydrazone linkage. In a detailed embodiment, the antibody specifically binds to an activated integrin receptor. In a further detailed embodiment, the activated integrin receptor is differentially produced on a cell in a metastatic state as compared to a similar, non-metastatic cell. In a further detailed embodiment, the activated integrin receptor is an α₃β₁ integrin receptor or an α₃β₁ integrin receptor. In a further detailed embodiment, the antibody is a single chain Fv antibody.

In a further embodiment, the method for treating a neoplastic disease in a mammal comprises internalizing the antibody-antitumor antibiotic conjugate within a cell of the mammal with cleavage of an acid-labile hydrazone linkage.

In another embodiment, a method of synthesizing an antibody-cytotoxin conjugate molecule comprises introducing into a single vessel an antibody, a thiolating reagent, and a maleimide-derivatized cytotoxin, contacting the antibody with the thiolating reagent to form a thiolated antibody, and contacting the thiolated antibody with the maleimide-derivatized cytotoxin to form an antibody-cytotoxin conjugate molecule. In a detailed aspect, the maleimide-derivatized cytotoxin comprises an acid-labile hydrazone linkage between maleimide and the cytotoxin. In a further detailed aspect, the maleimide-derivatized cytotoxin comprises an amide bond linkage between maleimide and the cytotoxin. In a further detailed aspect, the cytotoxin is an antitumor antibiotic, duocarmycin, duocarmycin A, duocarmycin SA, or an analog thereof. In a further detailed embodiment, the antitumor antibiotic is a carbonyl-substituted CBI-indole analog of duocarmycin SA. In a further detailed aspect, the antitumor antibiotic is an amide-substituted CBI-indole analog of duocarmycin SA. In a further detailed aspect, the thiolating reagent is 2-iminothiolane. In a further detailed aspect, the antibody is a single chain Fv antibody.

In a further detailed embodiment, the maleimide-derivatized cytotoxin is l-[3-(N′-{l-[2-(l-Chloromethyl-5-hydroxy-l,2-dihydro-3H-benzo[e]indole-3-carbonyl)-1H-indol-5-yl]-ethylidene}-hydrazino)-3-oxo-1-propyl]maleimide. In a further detailed embodiment, the maleimide-derivatized cytotoxin is 3-[5-[1-{3-[3-(2,5-dioxo-2,5-dihydropyrrol-1-yl)propionylamino]propyl}indole-2-carbonyl]aminoindole-2-carbonyl]-(1-chloromethyl)-5-hydroxy-1,2-dihydro-3H-benzo[e]indole.

In another embodiment, a compound of Formula I:

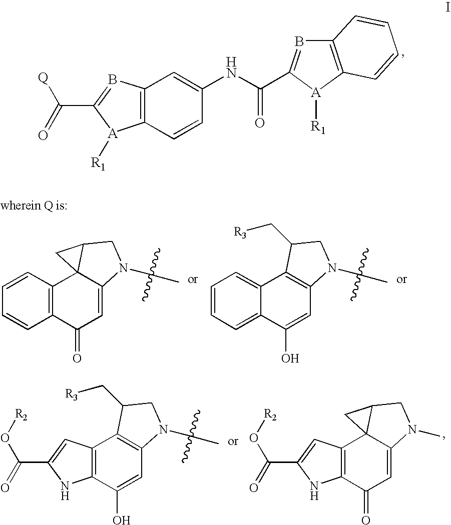

each A is independently NR₁, O or S, provided that at least one A is NR₁;

each B is independently C or N;

R₁ is independently H or —(CH₂)ₙ—N(H)R₄, provided that one R₁ is H and the other is —(CH₂)ₙ—N(H)R₅;

R₂ is alkyl;

R₃ is halogen;

R₄ is H or —C(═O)—(CH₂)ₘ—N-maleimide;

m is 2, 3, 4, 5 or 6; and

n is 2, 3, 4, 5 or 6;

or a stereoisomer, prodrug, pharmaceutically acceptable salt, hydrate, solvate, acid salt hydrate, N-oxide or isomorphic crystalline form thereof.

In a detailed embodiment, R₂ is C₁ to C₆ alkyl. In a further detailed embodiment, halogen is Cl, Br, or F.

In a further embodiment, a pharmaceutical composition comprises at least one pharmaceutically acceptable carrier or excipient and an effective amount of the compound of Formula I, wherein the maleimide moiety is conjugated to a single chain Fv antibody. In a detailed aspect, the single chain Fv antibody is an antibody to an integrin receptor. In a further detailed aspect, the integrin receptor is an-α₃β₁ integrin receptor or an αvβ3 integrin receptor. In a further detailed embodiment, a method comprises administering to a mammal the composition of Formula I.

In a further detailed embodiment, a method for alleviating a disease state in a mammal believed to be responsive to treatment with an antibody conjugated to a amide-substituted CBI-indole analog of duocarmycin SA comprises the step of administering to the mammal a therapeutic amount of the composition of Formula I. In a further detailed embodiment, the disease state is neoplastic disease.

In another embodiment, a compound is 3-[5-(1-(3-aminopropyl)indole-2-carbonyl)aminoindole-2-carbonyl]-1-(chloromethyl)-5-hydroxy-1,2-dihydro-3H-benz[e]indole.

In another embodiment, a compound is 3-[5-(1-(3-aminopropyl)indole-2-carbonyl)aminoindole-2-carbonyl]-1-(chloromethyl)-5-hydroxy-1,2-dihydro-3H-benz[e]indole.

In another embodiment, a compound is 3-[5-[1-{3-[3-(2,5-dioxo-2,5-dihydropyrrol-1-yl)propionylamino]propyl}indole-2-carbonyl]aminoindole-2-carbonyl]-(1-chloromethyl)-5-hydroxy-1,2-dihydro-3H-benzo[e]indole.

In another embodiment, a compound of Formula II:

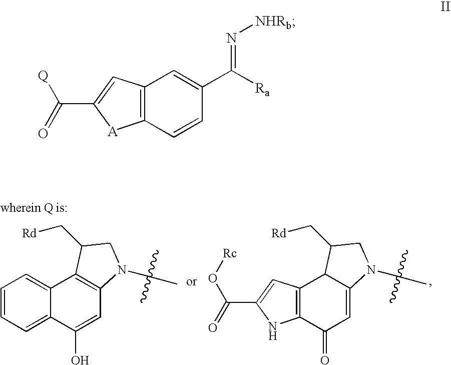

A is NH, O or S;

Rₐ is H or alkyl;

R_{b} is H, alkyl or —C(═O)—(CH₂)ᵣ—N-maleimide;

R_{c} is alkyl;

R_{d} is halogen; and

r is 2, 3, 4, 5 or 6;

or a stereoisomer, prodrug, pharmaceutically acceptable salt, hydrate, solvate, acid salt hydrate, N-oxide or isomorphic crystalline form thereof.

In another embodiment, a compound is 1-[3-(N′-{1-[2-(1-Chloromethyl-5-hydroxy-1,2-dihydro-3H-benzo[e]indole-3-carbonyl)-1H-indol-5-yl]-ethylidene}-hydrazino)-3-oxo-1-propyl]maleimide.

In a further embodiment, a pharmaceutical composition comprises at least one pharmaceutically acceptable carrier or excipient and an effective amount of the compound of Formula II, wherein the maleimide moiety is conjugated to a single chain Fv antibody. In a detailed aspect, the single chain Fv antibody is an antibody to an integrin receptor. In a further detailed aspect, the integrin receptor is an-α₃β₁ integrin receptor or an αvβ3 integrin receptor.

In a further embodiment, a method comprises administering to a mammal the composition of Formula II.

In a further embodiment, a method for alleviating a disease state in a mammal believed to be responsive to treatment with an antibody conjugated to a carbonyl-substituted CBI-indole analog of duocarmycin SA comprises the step of administering to the mammal a therapeutic amount of the composition of Formula II. In a detailed aspect, the disease state is neoplastic disease.

### BRIEF DESCRIPTION OF THE DRAWINGS

FIG. 1 Duocarmycin SA, CBI indole analogs and maleimide derivatives.

FIGS. 2A-2B SDS-PAGE of purified Pan10 and Pan10 conjugates. Lane 1: Invitrogen prestained markers; Lane 2: Pan10 after separate thiolation and conjugation; Lane 3: Pan10 after one-pot thiolation and conjugation; Lane 4: unmodified Pan10.

Bands density analysis (AlphaEaseFC StandAlone Software). The shaded boxes contain data relative to the band corresponding to the monomeric scFv in each lane.

FIG. 3 Confocal microscopy, overlaid 488 nm and 568 nm images of SW1990 and HdFa cells treated with Pan10-FM. (A) SW1990 cells after 2 h incubation, (B) HdFa cells after 2 h, (C) SW1990 cells after 3 h and (D) HdFa cells after 3 hours.

FIG. 4 Inverted microscope images of SW1990. (A) Untreated cells. (B) Cells treated with Pan10-FM. (C) Cells treated with Pan10-4, (D) cells treated with Pan10-3. The enlarged images of two of the cells treated with scFv-drug conjugates show extensive vacuolization.

FIG. 5 Schematic for the synthesis of Boc-protected 1.

### DETAILED DESCRIPTION

The invention is generally related to methods for treating a neoplastic disease with an antibody-cytotoxin conjugate molecule, methods of synthesizing an antibody-cytotoxin conjugate molecule, and compounds that are useful as antibody-cytotoxin conjugate molecule or useful in the synthesis of these molecules. Chemically modified anti-integrin α₃β₁ scFv Pan10 containing free thiols can be conjugated to maleimide-derivatized analogs of the potent cytotoxic agent duocarmycin SA. Antibody Pan10 conjugates conserve the ability to penetrate cells expressing integrin α₃β₁. In particular Pan10-drug conjugates show excellent cytotoxic effects on pancreatic carcinoma cells in vitro. This first step is important considering the unique advantage of the scFv conjugates compared to the free drugs described herein, which are extremely potent but not clinically viable anticancer agents. The conjugates can deliver these drug molecules more specifically to the interior of cancer cells which overexpress integrin α₃β₁ and efficient delivery of antibody drug conjugates should allow for reduced therapeutic drug exposure and enhanced efficacy. Using such a strategy, experiments will further elaborate the potential for scFv-drug designs in cancer treatment.

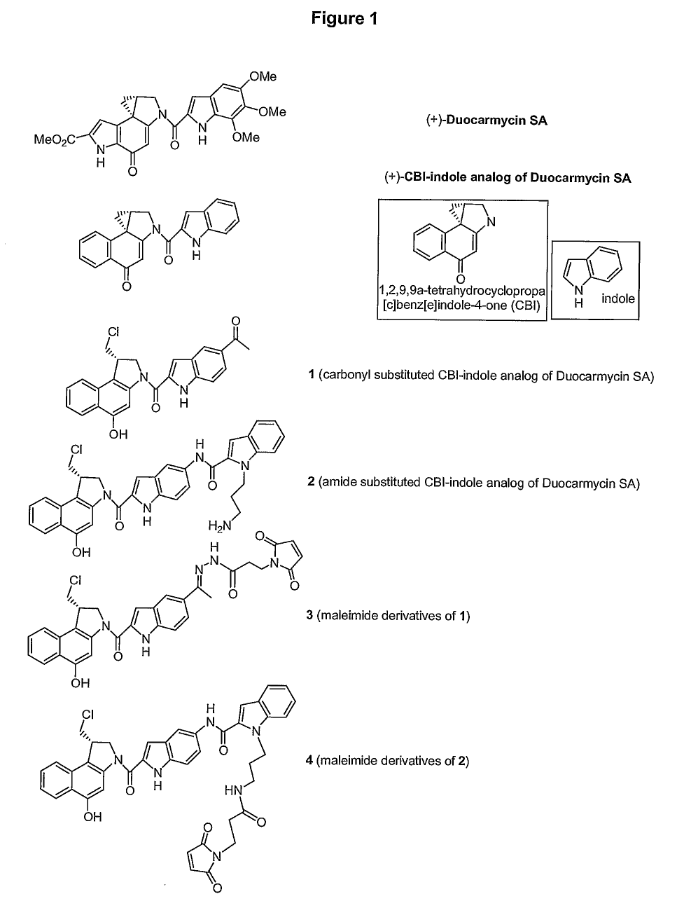

Biopanning of a human scFv-phage display library based on the selection requirement of internalization by the SW1990 human pancreatic adenocarcinoma cell line has been described. Gao et al., J Immunol Methods, 274: 185-197, 2003. A single chain Fv antibody (scFv), denoted Pan10, has been produced which upon immunoprecipitation, mass spectrometric analysis and database searching was found to target membrane receptor integrin α₃β₁. Because of the specific Pan10 interaction with α₃β₁ and the internalization capability, the Pan10 scFv can be a vector for conjugation with potent duocarmycin-SA analogs 3-(5-acetylindole-2-carbonyl)-1-(S)-(chloromethyl)-5-hydroxy-1,2-dihydro-3H-benz[e]indole (compound 1, FIG. 1) and 3-[5-(1-(3-aminopropyl)indole-2-carbonyl)aminoindole-2-carbonyl]-1-(chloromethyl)-5-hydroxy-1,2-dihydro-3H-benz[e]indole (compound 2, FIG. 1) to promote the destruction of malignant tumor cells overexpressing integrin α₃β₁. Parrish et al., Bioorg Med Chem, 11: 3815-3838, 2003. Maleimide-derivatized cytotoxins include, but are not limited to, 1-[3-(N′-{1-[2-(1-chloromethyl-5-hydroxy-1,2-dihydro-3H-benzo[e]indole-3-carbonyl)-1H-indol-5-yl]-ethylidene}-hydrazino)-3-oxo-1-propyl]maleimide (compound 3, FIG. 1), or 3-[5-[1-{3-[3-(2,5-dioxo-2,5-dihydropyrrol-1-yl)propionylamino]propyl}indole-2-carbonyl]aminoindole-2-carbonyl]-(1-chloromethyl)-5-hydroxy-1,2-dihydro-3H-benzo[e]indole (compound 4, FIG. 1). Hence, efforts have focused upon the following tasks, including: (a) The conjugation of antitumor drug(s) to scFv Pan10 without compromising target affinity and internalization properties. (b) The design of linkers promoting efficient attachment of the drug(s) to the scFv without compromising the cytotoxic activity of the drug(s). (c) The search for a reliable cell-based assay designed to evaluate the biological activity of Pan10-drug conjugates. The compounds and methods of the present invention provide a therapeutic application of scFv-mediated, tumor-targeted delivery of anti-cancer compounds.

It is to be understood that this invention is not limited to particular methods, reagents, compounds compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a cell” includes a combination of two or more cells, and the like.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described below are those well known and commonly employed in the art. Standard techniques are used for nucleic acid and peptide synthesis. Generally, enzymatic reactions and purification steps are performed according to the manufacturer's specifications. The techniques and procedures are generally performed according to conventional methods in the art and various general references (see generally, Sambrook et al. MOLECULAR CLONING: A LABORATORY MANUAL, 2d ed. (1989) Cold Spring Harbor Laboratory Press, Cold Spring Harbor, N.Y., which is incorporated herein by reference), which are provided throughout this document. The nomenclature used herein and the laboratory procedures in analytical chemistry, and organic synthetic described below are those well known and commonly employed in the art. Standard techniques, or modifications thereof, are used for chemical syntheses and chemical analyses. The term “therapeutic agent” is intended to mean a compound that, when present in a therapeutically effective amount, produces a desired therapeutic effect on a mammal. For treating carcinomas, it is desirable that the therapeutic agent also be capable of entering the target cell. Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“Neoplastic disease” refers to a disease resulting from uncontrolled growth of cells. Types of malignant neoplastic disease include, but are not limited to, carcinomas, sarcomas, leukemias, and lymphomas.

“Neoplastic cells” and “neoplasia” refer to cells which exhibit relatively autonomous growth, so that they exhibit an aberrant growth phenotype characterized by a significant loss of control of cell proliferation. Neoplastic cells comprise cells which can be actively replicating or in a temporary non-replicative resting state (G1 or G0); similarly, neoplastic cells can comprise cells which have a well-differentiated phenotype, a poorly-differentiated phenotype, or a mixture of both type of cells. Thus, not all neoplastic cells are necessarily replicating cells at a given timepoint. The set defined as neoplastic cells consists of cells in benign neoplasms and cells in malignant (or frank) neoplasms. Frankly neoplastic cells are frequently referred to as cancer (discussed supra), typically termed carcinoma if originating from cells of endodermal or ectodermal histological origin, or sarcoma if originating from cell types derived from mesoderm.

Elevated expression of integrin α3β1 has been observed in several types of metastatic neoplastic disease, for example malignant melanoma, bladder cancer, ocular melanocyte and uveal melanoma, and prostate carcinoma. Elevated expression of integrin αvβ3 has been observed in neoplastic disease, for example, malignant breast carcinoma. Further examples of neoplastic disease that can be treated by compositions of the present invention include, but are not limited to, solid tumor, hematological malignancy, leukemia, colorectal cancer, benign or malignant breast cancer, uterine cancer, uterine leiomyomas, ovarian cancer, endometrial cancer, polycystic ovary syndrome, endometrial polyps, squamous cell carcinoma, squamous cell carcinoma of the head and neck, hepatocellular carcinoma, intrahepatic metastasis of hepatocellular carcinoma, prostate cancer, prostatic hypertrophy, pituitary cancer, adenomyosis, adenocarcinoma, pancreatic adenocarcinoma, meningioma, melanoma, bone cancer, multiple myeloma, CNS cancer, glioma, or astroblastoma.

The term “cytotoxin” is intended to mean a therapeutic agent having the desired effect of being cytotoxic to cancer cells. Exemplary cytotoxins include, by way of example and not limitation, combretastatins, duocarmycins, the CC-1065 anti-tumor antibiotics, anthracyclines, and related compounds. Other cytotoxins include mycotoxins, ricin and its analogues, calicheamycins, doxirubicin and maytansinoids.

“Acid stable covalent linkage” refers to a covalent linkage between an antibody and a cytotoxin that is stable in a intracellular environment, when an antibody-cytotoxin conjugate enters a cell, for example, by receptor mediated endocytosis. The acid stable covalent linkage is usually not cleaved when the antibody-cytotoxin conjugate enters the cell. An amide linkage between antibody and cytotoxin is an example of an acid stable covalent linkage.

“Acid labile covalent linkage” refers to a cleavable covalent linkage between an antibody and a cytotoxin that is not stable in a intracellular environment, when an antibody-cytotoxin conjugate enters a cell, for example, by receptor mediated endocytosis. A hydrazone linkage between antibody and cytotoxin is an example of an acid labile covalent linkage.

The term “marker” is intended to mean a compound useful in the characterization of tumors or other medical condition, for example, diagnosis, progression of a tumor, and assay of the factors secreted by tumor cells. Markers are considered a subset of “diagnostic agents.”

“Inhibitors,” “activators,” and “modulators” of activated integrin receptor on metastatic cells are used to refer to inhibitory, activating, or modulating molecules, respectively, identified using in vitro and in vivo assays for integrin receptor binding or signaling, e.g., ligands, agonists, antagonists, and their homologs and mimetics.

The term “modulator” includes inhibitors and activators. Inhibitors are agents that, e.g., bind to, partially or totally block stimulation, decrease, prevent, delay activation, inactivate, desensitize, or down regulate the activity of activated integrin receptors, e.g., antagonists. Activators are agents that, e.g., bind to, stimulate, increase, open, activate, facilitate, enhance activation, sensitize or up regulate the activity of activated integrin receptors, e.g., agonists. Modulators include agents that, e.g., alter the interaction of activated integrin receptor with: proteins that bind activators or inhibitors, receptors, including proteins, peptides, lipids, carbohydrates, polysaccharides, or combinations of the above, e.g., lipoproteins, glycoproteins, and the like. Modulators include genetically modified versions of naturally-occurring activated integrin receptor ligands, e.g., with altered activity, as well as naturally occurring and synthetic ligands, antagonists, agonists, small chemical molecules and the like.

Such assays for inhibitors and activators include, e.g., applying putative modulator compounds to a cell expressing an activated integrin receptor and then determining the functional effects on integrin receptor signaling, as described herein. Samples or assays comprising activated integrin receptor that are treated with a potential activator, inhibitor, or modulator are compared to control samples without the inhibitor, activator, or modulator to examine the extent of inhibition. Control samples (untreated with inhibitors) can be assigned a relative integrin receptor activity value of 100%. Inhibition of activated integrin receptor is achieved when the integrin receptor activity value relative to the control is about 80%, optionally 50% or 25-0%. Activation of integrin receptor is achieved when the integrin receptor activity value relative to the control is 110%, optionally 150%, optionally 200-500%, or 1000-3000% higher.

The term “targeting group” is intended to mean a moiety that is (1) able to direct the entity to which it is attached (e.g., therapeutic agent or marker) to a target cell, for example to a specific type of tumor cell or (2) is preferentially activated at a target tissue, for example a tumor. The targeting group can be a small molecule, which is intended to include both non-peptides and peptides. The targeting group can also be a macromolecule, which includes saccharides, lectins, receptors, ligand for receptors, proteins such as BSA, antibodies, and so forth.

The term “cleavable group” or “cleavable linkage” is intended to mean a moiety that is unstable in vivo. Preferably the “cleavable group” or “cleavable linkage” allows for activation of the marker or therapeutic agent by cleaving the marker or agent from the rest of the conjugate. Operatively defined, the linker is preferably cleaved in vivo by the biological environment. The cleavage can come from any process without limitation, e.g., enzymatic, reductive, pH, and the like. Preferably, the cleavable group is selected so that activation occurs at the desired site of action, which can be a site in or near the target cells (e.g., carcinoma cells) or tissues such as at the site of therapeutic action or marker activity. Such cleavage is enzymatic and exemplary enzymatically cleavable groups include natural amino acids or peptide sequences that end with a natural amino acid, and are attached at their carboxyl terminus to the linker. While the degree of cleavage rate enhancement is not critical to the invention, preferred examples of cleavable linkers are those in which at least about 10% of the cleavable groups are cleaved in the blood stream within 24 hours of administration, most preferably at least about 35%. Preferred cleavable groups are peptide bonds, ester linkages, and disulfide linkages.

The symbol,

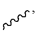

whether utilized as a bond or displayed perpendicular to a bond indicates the point at which the displayed moiety is attached to the remainder of the molecule, solid support, and the like.

The term “alkyl”, by itself or as part of another substituent, means, unless otherwise stated, a straight or branched chain, or cyclic hydrocarbon radical, or combination thereof, which can be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl”, unless otherwise noted, is also meant to include those derivatives of alkyl defined in more detail below, such as “heteroalkyl.” Alkyl groups, which are limited to hydrocarbon groups are termed “homoalkyl”.

The term “alkylene” by itself or as part of another substituent means a divalent radical derived from an alkane, as exemplified, but not limited, by CH₂CH₂CH₂CH₂, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “heteroalkyl”, by itself or in combination with another term, means, unless otherwise stated, a stable straight or branched chain, or cyclic hydrocarbon radical, or combinations thereof, consisting of the stated number of carbon atoms and at least one heteroatom selected from the group consisting of O, N, Si and S, and wherein the nitrogen, carbon and sulfur atoms can optionally be oxidized and the nitrogen heteroatom can optionally be quaternized. The heteroatom(s) O, N and S and Si can be placed at any interior position of the heteroalkyl group or at the position at which the alkyl group is attached to the remainder of the molecule. Examples include, but are not limited to, CH₂CH₂OCH₃, CH₂CH₂NHCH₃, CH₂CH₂N(CH₃)CH₃, —CH₂SCH₂—CH₃, CH₂CH₂, S(O)CH₃, CH₂CH₂S(O)₂CH₃, CH═CHOCH₃, Si(CH₃)₃, —CH₂CH═NOCH₃, and CH═CHN(CH₃)CH₃. Up to two heteroatoms can be consecutive, such as, for example, CH₂NHOCH₃ and CH₂OSi(CH₃)₃. Similarly, the term “heteroalkylene” by itself or as part of another substituent means a divalent radical derived from heteroalkyl, as exemplified, but not limited by, CH₂CH₂SCH₂CH₂ and CH₂SCH₂CH₂NHCH₂. For heteroalkylene groups, heteroatoms can also occupy either or both of the chain termini (e.g., alkyleneoxy, alkylenedioxy, alkyleneamino, alkylenediamino, and the like). The terms “heteroalkyl” and “heteroalkylene” encompass poly(ethylene glycol) and its derivatives (see, for example, Shearwater Polymers Catalog, 2001). Still further, for alkylene and heteroalkylene linking groups, no orientation of the linking group is implied by the direction in which the formula of the linking group is written. For example, the formula C(O)₂R′ represents both —C(O)₂R′ and R′C(O)₂.

The term “lower” in combination with the terms “alkyl” or “heteroalkyl” refers to a moiety having from 1 to 6 carbon atoms.

The terms “alkoxy”, “alkylamino” and “alkylthio” (or thioalkoxy) are used in their conventional sense, and refer to those alkyl groups attached to the remainder of the molecule via an oxygen atom, an amino group, or a sulfur atom, respectively.

In general, an “acyl substituent” is also selected from the group set forth above. As used herein, the term “acyl substituent” refers to groups attached to, and fulfilling the valence of a carbonyl carbon that is either directly or indirectly attached to the polycyclic nucleus of the compounds of the present invention.

The terms “cycloalkyl” and “heterocycloalkyl”, by themselves or in combination with other terms, represent, unless otherwise stated, cyclic versions of substituted or unsubstituted “alkyl” and substituted or unsubstituted “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropy-ridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The heteroatoms and carbon atoms of the cyclic structures are optionally oxidized.

The terms “halo” or “halogen”, by themselves or as part of another substituent, mean, unless otherwise stated, a fluorine, chlorine, bromine, or iodine atom. Additionally, terms such as “haloalkyl”, are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a substituted or unsubstituted polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen, carbon and sulfur atoms are optionally oxidized, and the nitrogen atom(s) are optionally quaternized. A heteroaryl group can be attached to the remainder of the molecule through a heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. “Aryl” and “heteroaryl” also encompass ring systems in which one or more non-aromatic ring systems are fused, or otherwise bound, to an aryl or heteroaryl system.

For brevity, the term “aryl” when used in combination with other terms (e.g., aryloxy, arylthioxy, and arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl”, “heteroalkyl”, “aryl” and “heteroaryl”) include both substituted and unsubstituted forms of the indicated radical. Preferred substituents for each type of radical are provided below.

Substituents for the alkyl, and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generally referred to as “alkyl substituents” and “heteroalkyl substituents”, respectively, and they can be one or more of a variety of groups selected from, but not limited to: OR′, ═O, ═NR′, ═NOR′, NR′R″, SR′, -halogen, SiR′R″R′″, OC(O)R′, C(O)R′, CO₂R′, —CONR′R″, OC(O)NR′R″, NR″C(O)R′, NR′—C(O)NR″R′″, NR″C(O)₂R′, NR—C(NR′R″R′″)═NR″″, NRC(NR′R″)═NR′″, S(O)R′, S(O)₂R′, S(O)₂NR′R″, NRSO₂R′, CN and NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., CF₃ and CH₂CF₃) and acyl (e.g., C(O)CH₃, C(O)CF₃, C(O)CH₂OCH₃, and the like).

Similar to the substituents described for the alkyl radical, the aryl substituents and heteroaryl substituents are generally referred to as “aryl substituents” and “heteroaryl substituents”, respectively and are varied and selected from, for example: halogen, OR′, ═O, ═NR′, ═NOR′, NR′R″, SR′, -halogen, SiR′R″R′″, OC(O)R′, C(O)R′, —CO₂R′, CONR′R″, OC(O)NR′R″, NR″C(O)R′, NR′C(O)NR″R′″, NR″C(O)₂R′, NR—C(NR′R″)═NR′″, S(O)R′, S(O)₂R′, S(O)₂NR′R″, NRSO₂R′, CN and NO₂, R′, N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, (C₁-C₈)alkyl and heteroalkyl, unsubstituted aryl and heteroaryl, (unsubstituted aryl)-(C₁-C₄)alkyl, and (unsubstituted aryl)oxy-(C₁-C₄)alkyl. When a compound of the invention includes more than one R group, for example, each of the R groups is independently selected as are each R′, R″, R′″ and R″″ groups when more than one of these groups is present.

Two of the aryl substituents on adjacent atoms of the aryl or heteroaryl ring can optionally be replaced with a substituent of the formula -T-C(O)(CRR′)_{q}U, wherein T and U are independently NR, O, CRR′ or a single bond, and q is an integer of from 0 to 3. Alternatively, two of the substituents on adjacent atoms of the aryl or heteroaryl ring can optionally be replaced with a substituent of the formula -A-(CH₂)ᵣ—B, wherein A and B are independently CRR′, O, NR, S, S(O), S(O)₂, S(O)₂NR′ or a single bond, and r is an integer of from 1 to 4. One of the single bonds of the new ring so formed can optionally be replaced with a double bond. Alternatively, two of the substituents on adjacent atoms of the aryl or heteroaryl ring can optionally be replaced with a substituent of the formula (CRR′)ₛX(CR″R′″)_{d}, where s and d are independently integers of from 0 to 3, and X is O, NR′, S, S(O), S(O)₂, or S(O)₂NR′. The substituents R, R′, R″ and R′″ are preferably independently selected from hydrogen or substituted or unsubstituted (C₁-C₆)alkyl.

As used herein, the term “heteroatom” includes oxygen (O), nitrogen (N), sulfur (S) and silicon (Si).

The neutral forms of the compounds are preferably regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention can exist in multiple crystalline or amorphous forms. In general, all physical forms are equivalent for the uses contemplated by the present invention and are intended to be within the scope of the present invention.

The compounds of the present invention can also contain unnatural proportions of atomic isotopes at one or more of the atoms that constitute such compounds. For example, the compounds can be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

The term “attaching moiety” or “moiety for attaching a targeting group” refers to a moiety which allows for attachment of a targeting group to the linker. Typical attaching groups include, by way of illustration and not limitation, alkyl, aminoalkyl, aminocarbonylalkyl, carboxyalkyl, hydroxyalkyl, alkyl-maleimide, alkyl-N-hydroxylsuccinimide, poly(ethylene glycol)-maleimide and poly(ethylene glycol)-N-hydroxylsuccinimide, all of which can be further substituted. The linker can also have the attaching moiety be actually appended to the targeting group.

As used herein, the term “leaving group” refers to a portion of a substrate that is cleaved from the substrate in a reaction.

“Solid support”, as used herein refers to a material that is substantially insoluble in a selected solvent system, or which can be readily separated (e.g., by precipitation) from a selected solvent system in which it is soluble. Solid supports useful in practicing the present invention can include groups that are activated or capable of activation to allow selected species to be bound to the solid support. A solid support can also be a substrate, for example, a chip, wafer or well, onto which an individual, or more than one compound, of the invention is bound.

“Reactive functional group”, as used herein refers to groups including, but not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those used to prepare bioconjugates, e.g., N-hydroxysuccinimide esters, maleimides and the like (see, for example, Hermanson, BIOCONJUGATE TECHNIQUES, Academic press, San Diego, 1996). Methods to prepare each of these functional groups are well known in the art and their application to or modification for a particular purpose is within the ability of one of skill in the art (see, for example, Sandier and Karo, eds. ORGANIC FUNCTIONAL GROUP PREPARATIONS, Academic Press, San Diego, 1989).

Certain compounds of the present invention possess asymmetric carbon atoms (optical centers) or double bonds; the racemates, diastereomers, geometric isomers and individual isomers are encompassed within the scope of the present invention. The compounds of the invention are prepared as a single isomer (e.g., enantiomer, cis-trans, positional, diastereomer) or as a mixture of isomers. In a preferred embodiment, the compounds are prepared as substantially a single isomer. Methods of preparing substantially isomerically pure compounds are known in the art. For example, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Alternatively, the final product or intermediates along the synthetic route can be resolved into a single stereoisomer. Techniques for inverting or leaving unchanged a particular stereocenter, and those for resolving mixtures of stereoisomers are well known in the art and it is well within the ability of one of skill in the art to choose and appropriate method for a particular situation. See, generally, Fumiss et al. (eds.), VOGEL'S ENCYCLOPEDIA OF PRACTICAL ORGANIC CHEMISTRY 5^{th} ED., Longman Scientific and Technical Ltd., Essex, 1991, pp. 809-816; and Heller, Acc. Chem. Res. 23: 128 (1990).

Immunotoxins

The invention is directed to methods of synthesizing an antibody-cytotoxin conjugate molecule, and compounds that are useful as antibody-cytotoxin conjugate molecules, or immunotoxins, or useful in the synthesis of these molecules. This invention is also directed to immunochemical derivatives of the antibodies, such as immunotoxins. Antibodies which carry the appropriate effector functions, such as with their constant domains, are also used to induce lysis through the natural complement process, and to interact with antibody dependent cytotoxic cells normally present. For example, purified, sterile filtered antibodies are optionally conjugated to a cytotoxin such as duocarmycin for use in cancer therapy. The methods of this invention, for example, are suitable for obtaining humanized antibodies for use as immunotoxins for use in cancer therapy.

The methods of the present invention provide antibody-cytotoxin conjugate molecules that utilize antibodies to direct potent cytotoxic drugs directly to tumors in a highly selective way, thus reducing indiscriminate cell destruction. These methods will potentially enhance the efficacy of, and also reduce the side effects frequently associated with, chemotherapeutic agents.

The cytotoxic moiety of the immunotoxin can be a cytotoxic drug or an enzymatically active toxin of bacterial, fungal, plant or animal origin, or an enzymatically active fragment of such a toxin. Enzymatically active toxins and fragments thereof include, but are not limited to, duocarmycin and analogs thereof. Enzymatically active toxins and fragments thereof further include, but are not limited to, diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecens.

“Antibody-cytotoxin conjugate” is formed when antibodies are conjugated to small molecule anticancer drugs such as cis-platin or 5-fluorouracil. Conjugates of the monoclonal antibody and such cytotoxic moieties can be made using a variety of bifunctional protein coupling agents. Examples of such reagents are SPDP, IT, bifunctional derivatives of imidoesters such as dimethyl adipimidate HCl, active esters such as disuccinimidyl suberate, aldehydes such as glutaraldehyde, bis-azido compounds such as bis (p-azidobenzoyl) hexanediamine, bis-diazonium derivatives such as bis-(p-diazoniumbenzoyl)-ethylenediamine, diisocyantes such as tolylene 2,6-diisocyanate are bis-active fluorine compounds such as 1,5-difluoro-2,4-dinitrobenzene. The lysing portion of a toxin can be joined to the Fab fragment of the antibodies.

Immunotoxins can be made in a variety of ways, as discussed herein. Commonly known crosslinking reagents can be used to yield stable conjugates.

Advantageously, monoclonal antibodies specifically binding the domain of the antigen which is exposed on the infected cell surface, are conjugated to ricin A chain. Most advantageously the ricin A chain is deglycosylated and produced through recombinant means. An advantageous method of making the ricin immunotoxin is described in Vitetta et al., Science 238:1098 (1987).

When used to kill infected human cells in vitro for diagnostic purposes, the conjugates will typically be added to the cell culture medium at a concentration of at least about 10 nM. The formulation and mode of administration for in vitro use are not critical. Aqueous formulations that are compatible with the culture or perfusion medium will normally be used. Cytotoxicity can be read by conventional techniques.

Cytotoxic radiopharmaceuticals for treating infected cells can be made by conjugating radioactive isotopes (e.g., I, Y, Pr) to the antibodies. Advantageously alpha particle-emitting isotopes are used. The term “cytotoxic moiety” as used herein is intended to include such isotopes.

In a further embodiment toxin-conjugates are made with Fab or F(ab′)₂ fragments. Because of their relatively small size these fragments can better penetrate tissue to reach infected cells.

In another embodiment, fusogenic liposomes are filled with a cytotoxic drug and the liposomes are coated with antibodies specifically binding the particular antigen.

Antibodies and their Uses

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding.

The present invention further relates to antibodies and T-cell antigen receptors (TCR) which specifically bind the polypeptides of the present invention. The antibodies of the present invention include IgG (including IgG₁, IgG₂, IgG₃, and IgG₄), IgA (including IgA₁ and IgA₂), IgD, IgE, or IgM, and IgY. As used herein, the term “antibody” (Ab) is meant to include whole antibodies, including single-chain whole antibodies, and antigen-binding fragments thereof. Most preferably the antibodies are human antigen binding antibody fragments of the present invention and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. The antibodies can be from any animal origin including birds and mammals. Preferably, the antibodies are human, murine, rabbit, goat, guinea pig, camel, horse, or chicken.

Antigen-binding antibody fragments, including single-chain antibodies, can comprise the variable region(s) alone or in combination with the entire or partial of the following: hinge region, CH₁, CH₂, and CH₃ domains. Also included in the invention are any combinations of variable region(s) and hinge region, CH₁, CH₂, and CH₃ domains. The present invention further includes monoclonal, polyclonal, chimeric, humanized, and human monoclonal and human polyclonal antibodies which specifically bind the polypeptides of the present invention. The present invention further includes antibodies which are anti-idiotypic to the antibodies of the present invention.

The antibodies of the present invention can be monospecific, bispecific, trispecific or of greater multispecificity. Multispecific antibodies can be specific for different epitopes of a polypeptide of the present invention or can be specific for both a polypeptide of the present invention as well as for heterologous compositions, such as a heterologous polypeptide or solid support material. See, e.g., WO 93/17715; WO 92/08802; WO 91/00360; WO 92/05793; Tutt et al., J. Immunol. 147: 60-69, 1991; U.S. Pat. Nos. 5,573,920, 4,474,893, 5,601,819, 4,714,681, 4,925,648, each incorporated herein by reference in their entirety and for all purposes; Kostelny et al., J. Immunol. 148: 1547-1553, 1992.

An intact “antibody” comprises at least two heavy (H) chains and two light (L) chains inter-connected by disulfide bonds. Each heavy chain is comprised of a heavy chain variable region (abbreviated herein as HCVR or V_{H}) and a heavy chain constant region. The heavy chain constant region is comprised of three domains, CH₁, CH₂ and CH₃. Each light chain is comprised of a light chain variable region (abbreviated herein as LCVR or V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxyl-terminus in the following order: FR₁, CDR₁, FR₂, CDR₂, FR₃, CDR₃, FR₄. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system. The term antibody includes antigen-binding portions of an intact antibody that retain capacity to bind activated integrin receptor. Examples of binding include (i) a Fab fragment, a monovalent fragment consisting of the V_{L}, V_{H}, C_{L} and CH₁ domains; (ii) a F(ab′)₂ fragment, a bivalent fragment comprising two Fab fragments linked by a disulfide bridge at the hinge region; (iii) a Fd fragment consisting of the V_{H} and CH₁ domains; (iv) a Fv fragment consisting of the V_{L} and V_{H} domains of a single arm of an antibody, (v) a dAb fragment (Ward et al., Nature 341: 544-546, 1989), which consists of a V_{H} domain; and (vi) an isolated complementarity determining region (CDR).

An “isolated” antibody is one that has been identified and separated and for recovered from a component of its natural environment. Contaminant components of its natural environment are materials that would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient too obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant 15 cells since at least one component of the antibody's natural environment will not be present. Ordinarily, however, an isolated antibody will be prepared by at least one purification step.

“Single chain antibodies” or “single chain Fv (scFv)” refers to an antibody fusion molecule of the two domains of the Fv fragment, V_{L} and V_{H}. Although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); See, e.g., Bird et al., Science 242: 423-426, 1988; and Huston et al., Proc. Natl. Acad. Sci. USA, 85: 5879-5883, 1988). Such single chain antibodies are included by reference to the term “antibody” fragments, and can be prepared by recombinant techniques or enzymatic or chemical cleavage of intact antibodies.

“Human sequence antibody” includes antibodies having variable and constant regions (if present) derived from human germline immunoglobulin sequences. The human sequence antibodies of the invention can include amino acid residues not encoded by human germline immunoglobulin sequences (e.g., mutations introduced by random or site-specific mutagenesis in vitro or by somatic mutation in vivo). Such antibodies can be generated in non-human transgenic animals, e.g., as described in PCT Publication Nos. WO 01/14424 and WO 00/37504. However, the term “human sequence antibody”, as used herein, is not intended to include antibodies in which CDR sequences derived from the germline of another mammalian species, such as a mouse, have been grafted onto human framework sequences (e.g., humanized antibodies).

Also, recombinant immunoglobulins can be produced. See, Cabilly, U.S. Pat. No. 4,816,567 incorporated herein by reference in its entirety and for all purposes; and Queen et al., Proc. Nat'l Acad. Sci. USA 86: 10029-10033, 1989.

“Monoclonal antibody” refer to a preparation of antibody molecules of single molecular composition. A monoclonal antibody composition displays a single binding specificity and affinity for a particular epitope. Accordingly, the term “human monoclonal antibody” refers to antibodies displaying a single binding specificity which have variable and constant regions (if present) derived from human germline immunoglobulin sequences. In one embodiment, the human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

The term “monoclonal antibody” is not limited to antibodies produced through hybridoma technology. The term “monoclonal antibody” refers to an antibody that is derived from a single clone, including any eukaryotic, prokaryotic, or phage clone, and not the method by which it is produced. Monoclonal antibodies can be prepared using a wide variety of techniques known in the art including the use of hybridoma, recombinant, and phage display technology.

“Polyclonal antibody” refers to a preparation of more than 1 (two or more) different antibodies to a cell surface receptor, e.g., human activated integrin receptor. Such a preparation includes antibodies binding to a range of different epitopes.

“Chimeric antibodies” are those in which the Fc constant region of a monoclonal antibody from one species (typically a mouse) is replaced, using recombinant DNA techniques, with an Fc region from an antibody of another species (typically a human). For example, a cDNA encoding a murine monoclonal antibody is digested with a restriction enzyme selected specifically to remove the sequence encoding the Fc constant region, and the equivalent portion of a cDNA encoding a human Fc constant region is substituted (see Robinson et al., PCT/US86/02269; Akira et al., European Patent Application 184,187; Taniguchi, European Patent Application 171,496; Morrison et al., European Patent Application 173,494; Neuberger et al., WO 86/01533; Cabilly et al. U.S. Pat. No. 4,816,567; Cabilly et al., European Patent Application 125,023; Better et al. (1988) Science 240:1041-1043; Liu et al. (1987) Proc Natl Acad Sci USA 84:3439-3443; Liu et al. (1987) J Immunol 139:3521-3526; Sun et al. (1987) Proc Natl Acad Sci USA 84:214-218; Nishimura et al. (1987) Cancer Res 47:999-1005; Wood et al. (1985) Nature 314:446-449; and Shaw et al. (1988) J Natl Cancer Inst 80:1553-1559).

A CDR-grafted antibody is an antibody in which at least one CDR of a so-called “acceptor” antibody is replaced by a CDR “graft” from a so-called “donor” antibody possessing a desirable antigen specificity. Generally the donor and acceptor antibodies are monoclonal antibodies from different species; typically the acceptor antibody is a human antibody (to minimize its antigenicity in a human), in which case the resulting CDR-grafted antibody is termed a “humanized” antibody. The graft may be of a single CDR (or even a portion of a single CDR) within a single VH or VL of the acceptor antibody, or can be of multiple CDRs (or portions thereof) within one or both of the VH and VL. Frequently all three CDRs in all variable domains of the acceptor antibody will be replaced with the corresponding donor CDRs, though one need replace only as many as necessary to permit adequate binding of the resulting CDR-grafted antibody to MetAp3. Methods for generating CDR-grafted and humanized antibodies are taught by Queen et al. U.S. Pat. No. 5,585,089, U.S. Pat. No. 5,693,761 and U.S. Pat. No. 5,693,762; and Winter U.S. Pat. No. 5,225,539, the contents of all of which are hereby incorporated by reference.

This process typically does not alter the acceptor antibody's FRs flanking the grafted CDRs. However, one can sometimes improve antigen binding affinity of the resulting CDR grafted antibody by replacing certain residues of a given FR to make the FR more similar to the corresponding FR of the donor antibody. Preferred locations of the substitutions include amino acid residues adjacent to the CDR, or which are capable of interacting with a CDR (see, e.g., U.S. Pat. No. 5,585,089, especially columns 12-16). Or one can start with the donor FR and modify it to be more similar to the acceptor FR or a human consensus FR. Techniques for making these modifications are known in the art. Particularly if the resulting FR fits a human consensus FR for that position, or is at least 90% or more identical to such a consensus FR, doing so may not increase the antigenicity of the resulting modified antibody significantly compared to the same antibody with a fully human FR. As used herein, the term “consensus sequence” refers to the sequence formed from the most frequently occurring amino acids (or nucleotides) in a family of related sequences (See e.g., Winnaker, From Genes to Clones (Verlagsgesellschaft, Weinheim, Germany 1987). In a family of proteins, each position in the consensus sequence is occupied by the amino acid occurring most frequently at that position in the family. If two amino acids occur equally frequently, either can be included in the consensus sequence. A “consensus FR” refers to a FR in a consensus immunoglobulin sequence.

Antibodies to activated integrin receptor can bind to an epitope on human activated integrin receptor so as to inhibit activated integrin receptor from interacting with a counterreceptor or co-receptor. These and other antibodies suitable for use in the present invention can be prepared according to methods that are well known in the art and/or are described in the references cited here. In preferred embodiments, anti-activated integrin receptor antibodies used in the invention are “human antibodies”—e.g., antibodies isolated from a human—or they are “human sequence antibodies” (defined supra).

Antibodies of the present invention can be described or specified in terms of the epitope(s) or portion(s) of a polypeptide of the present invention which are recognized or specifically bound by the antibody. The epitope(s) or polypeptide portion(s) can be specified as described herein, e.g., by N-terminal and C-terminal positions, by size in contiguous amino acid residues. Antibodies which specifically bind any epitope or polypeptide of the present invention can also be excluded. Therefore, the present invention includes antibodies that specifically bind polypeptides of the present invention, and allows for the exclusion of the same.

“Epitope” refers to a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

Preferred epitopes of α₃β₁ are located on the surface of the folded protein, e.g., hydrophilic regions, as well as regions with high antigenicity. For example, an Emini surface probability analysis of the human α₃β₁ sequence can be used to indicate regions with a particularly high probability of being localized to the surface of the protein and thus likely to constitute epitopes useful for targeting antibody production.

Antibodies of the present invention can also be described or specified in terms of their cross-reactivity. Antibodies that do not bind any other analog, ortholog, or homolog of the polypeptides of the present invention are included. Antibodies that do not bind polypeptides with less than 95%, less than 90%, less than 85%, less than 80%, less than 75%, less than 70%, less than 65%, less than 60%, less than 55%, and less than 50% identity (as calculated using methods known in the art and described herein) to a polypeptide of the present invention are also included in the present invention. Further included in the present invention are antibodies which only bind polypeptides encoded by polynucleotides which hybridize to a polynucleotide of the present invention under stringent hybridization conditions (as described herein). Antibodies of the present invention can also be described or specified in terms of their binding affinity. Preferred binding affinities include those with a dissociation constant or K_{d} less than 5×10⁻⁶M, 10⁻⁶M, 5×10⁻⁷M, 10⁻⁷M, 5×10⁻⁸M, 10⁻⁸M, 5×10⁻⁹M, 10⁻⁹M, 5×10⁻¹⁰M, 10⁻¹⁰M, 5×10⁻¹¹M, 10⁻¹¹M, 5×10⁻¹²M, 10⁻¹²M, 5×10⁻¹³M, 10⁻¹³M, 5×10⁻¹⁴M, 10⁻¹⁴M, 5×10⁻¹⁵M, and 10⁻¹⁵M.

Antibodies to activated integrin receptors of the invention have uses that include, but are not limited to, methods known in the art to purify, detect, and target the polypeptides of the present invention including both in vitro and in vivo diagnostic and therapeutic methods. For example, the antibodies have use in immunoassays for qualitatively and quantitatively measuring levels of the polypeptides of the present invention in biological samples. See, e.g., Harlow & Lane, supra, incorporated herein by reference in its entirety and for all purposes.

The antibodies of the present invention can be used either alone or in combination with other compositions. The antibodies can further be recombinantly fused to a heterologous polypeptide at the N- or C-terminus or chemically conjugated (including covalently and non-covalently conjugations) to polypeptides or other compositions. For example, antibodies of the present invention can be recombinantly fused or conjugated to molecules useful as labels in detection assays and effector molecules such as heterologous polypeptides, drugs, or toxins. See, e.g., WO 92/08495; WO 91/14438; WO 89/12624; U.S. Pat. No. 5,314,995; and EP 0 396 387, each incorporated herein by reference in their entirety and for all purposes.

The present invention further includes compositions comprising the polypeptides of the present invention fused or conjugated to antibody domains other than the variable regions. For example, the polypeptides of the present invention can be fused or conjugated to an antibody Fc region, or portion thereof. The antibody portion fused to a polypeptide of the present invention can comprise the hinge region, CH₁ domain, CH₂ domain, and CH₃ domain or any combination of whole domains or portions thereof. The polypeptides of the present invention can be fused or conjugated to the above antibody portions to increase the in vivo half life of the polypeptides or for use in immunoassays using methods known in the art. The polypeptides can also be fused or conjugated to the above antibody portions to form multimers. For example, Fc portions fused to the polypeptides of the present invention can form dimers through disulfide bonding between the Fc portions. Higher multimeric forms can be made by fusing the polypeptides to portions of IgA and IgM. Methods for fusing or conjugating the polypeptides of the present invention to antibody portions are known in the art. See e.g., U.S. Pat. Nos. 5,336,603, 5,622,929, 5,359,046, 5,349,053, 5,447,851, 5,112,946; EP 0 307 434, EP 0 367 166; WO 96/04388, WO 91/06570, each incorporated herein by reference in their entirety and for all purposes; Ashkenazi et al., PNAS, 88: 10535-10539, 1991; Zheng et al., J. Immunol., 154: 5590-5600, 1995; and Vil et al., PNAS, 89: 11337-11341, 1992.

The invention further relates to antibodies which act as agonists or antagonists of the polypeptides of the present invention. For example, the present invention includes antibodies which disrupt the receptor/ligand interactions with the polypeptides of the invention either partially or fully. Included are both receptor-specific antibodies and ligand-specific antibodies. Included are receptor-specific antibodies which do not prevent ligand binding but prevent receptor activation. Receptor activation (i.e., signaling) can be determined by techniques described herein or otherwise known in the art. Also include are receptor-specific antibodies which both prevent ligand binding and receptor activation.

Likewise included are neutralizing antibodies which bind the ligand and prevent binding of the ligand to the receptor, as well as antibodies which bind the ligand, thereby preventing receptor activation, but do not prevent the ligand from binding the receptor. By “neutralizing antibody” is meant an antibody molecule that is able to eliminate or significantly reduce an effecter function of a target antigen to which is binds. Accordingly, a “neutralizing” anti-target antibody is capable of eliminating or significantly reducing an effecter function, such as enzyme activity, ligand binding, or intracellular signaling

Further included are antibodies which activate the receptor. These antibodies can act as agonists for either all or less than all of the biological activities affected by ligand-mediated receptor activation. The antibodies can be specified as agonists or antagonists for biological activities comprising specific activities disclosed herein. The above antibody agonists can be made using methods known in the art. See e.g., WO 96/40281; U.S. Pat. No. 5,811,097, each incorporated herein by reference in their entirety and for all purposes; Deng et al., Blood 92: 1981-1988, 1998; Chen et al., Cancer Res., 58: 3668-3678, 1998; Harrop et al., J. Immunol. 161: 1786-1794, 1998; Zhu et al., Cancer Res., 58: 3209-3214, 1998; Yoon et al., J. Immunol., 160: 3170-3179, 1998; Prat et al., J. Cell. Sci., 111: 237-247, 1998; Pitard et al., J. Immunol. Methods, 205: 177-190, 1997; Liautard et al., Cytokinde, 9: 233-241, 1997; Carlson et al., J. Biol. Chem., 272: 11295-11301, 1997; Taryman et al., Neuron, 14: 755-762, 1995; Muller et al., Structure, 6: 1153-1167, 1998; Bartunek et al., Cytokinem, 8: 14-20, 1996. As discussed above, antibodies to activated integrin receptors on metastatic cells can, in turn, be utilized to generate anti-idiotype antibodies that “mimic” polypeptides of the invention using techniques well known to those skilled in the art. (See, e.g., Greenspan et al., FASEB J. 7: 437-444, 1989 and Nissinoff, J. Immunol. 147: 2429-2438, 1991). For example, antibodies which bind to and competitively inhibit polypeptide multimerization and/or binding of a polypeptide of the invention to ligand can be used to generate anti-idiotypes that “mimic” the polypeptide multimerization and/or binding domain and, as a consequence, bind to and neutralize polypeptide and/or its ligand. Such neutralizing anti-idiotypes or Fab fragments of such anti-idiotypes can be used in therapeutic regimens to neutralize polypeptide ligand. For example, such anti-idiotypic antibodies can be used to bind a polypeptide of the invention and/or to bind its ligands/receptors, and thereby block its biological activity.

Antibody Preparation and Generation

Methods of generating antibodies or antibody fragments of the invention typically include immunizing a subject (generally a non-human subject such as a mouse or rabbit) with purified α₃β₁ or with a cell expressing α₃β₁. Any immunogenic portion of this polypeptide can be employed as the immunogen.

Typically, the immunogen will be at least 8 amino acyl residues in length, and preferably at least 10. Multimers of a given epitope are sometimes more effective than a monomer. If needed, the immunogenicity of the polypeptide can be increased by fusion or conjugation to a hapten such as keyhole limpet hemocyanin (KLH). Many such haptens are known in the art. Alternatively or in addition, one can combine the polypeptide with a conventional adjuvant such as Freund's complete or incomplete adjuvant to increase the subject's immune reaction to the polypeptide. These techniques are standard in the art.

Following appropriate immunization, polyclonal antibody that binds to α₃β₁ can be prepared from the subject's serum, or hybridomas expressing monoclonal antibodies can be prepared from the subject's spleen using routine methods. See, e.g., Milstein et al. (Galfre and Milstein, Methods Enzymol (1981) 73:3-46). Screening the hybridomas using standard methods will produce monoclonal antibodies of varying specificity (i.e., for different epitopes) and affinity. A selected monoclonal antibody with the desired properties can be used as expressed by the hybridoma, it can be bound to a molecule such as polyethylene glycol (PEG) to alter its properties, or a cDNA encoding it can be isolated, sequenced and manipulated in various ways. Examples of such manipulations were discussed above in the context of CDR grafting and FR modifications. Other manipulations include substituting or deleting particular amino acyl residues that contribute to instability of the antibody during storage or after administration to a patient, and affinity maturation techniques to improve affinity of the antibody for MetAp3.

Monoclonal antibodies also can be produced using recombinant techniques known in the art. For example, a population of nucleic acids that encode regions of antibodies can be isolated. PCR utilizing primers derived from sequences encoding conserved regions of antibodies is used to amplify sequences encoding portions of antibodies from the population and then reconstruct DNAs encoding antibodies or fragments thereof, such as variable domains, from the amplified sequences. Such amplified sequences also can be fused to DNAs encoding other proteins—for example, a bacteriophage coat, or a bacterial cell surface protein—for expression and display of the fusion polypeptides on phage or bacteria. Amplified sequences can then be expressed and further selected or isolated based, for example, on the affinity of the expressed antibody or fragment thereof for an antigen or epitope. Other methods for producing hybridomas and monoclonal antibodies are well known to those of skill in the art.

Hybridoma techniques include those known in the art and taught in Harlow & Lane, supra; Hammerling et al., Monoclonal Antibodies And T-Cell Hybridomas, 563-681, 1981, said references incorporated by reference in their entireties. Fab and F(ab′)₂ fragments can be produced by proteolytic cleavage, using enzymes such as papain (to produce Fab fragments) or pepsin (to produce F(ab′)₂ fragments).

Alternatively, antibodies to activated integrin receptor can be produced through the application of recombinant DNA and phage display technology or through synthetic chemistry using methods known in the art. For example, the antibodies of the present invention can be prepared using various phage display methods known in the art. In phage display methods, functional antibody domains are displayed on the surface of a phage particle which carries polynucleotide sequences encoding them. Phage with a desired binding property are selected from a repertoire or combinatorial antibody library (e.g., human or murine) by selecting directly with antigen, typically antigen bound or captured to a solid surface or bead. Phage used in these methods are typically filamentous phage including fd and M13 with Fab, Fv or disulfide stabilized Fv antibody domains recombinantly fused to either the phage gene III or gene Vil protein. Examples of phage display methods that can be used to make the antibodies of the present invention include those disclosed in Brinkman et al., J. Immunol. Methods 182: 41-50, 1995; Ames et al., J. Immunol. Methods 184: 177-186, 1995; Kettleborough et al., Eur. J. Immunol. 24: 952-958, 1994; Persic et al., Gene 187: 9-18, 1997; Burton et al., Advances in Immunology 57: 191-280, 1994; PCT/GB91/01134; WO 90/02809; WO 91/10737; WO 92/01047; WO 92/18619; WO 93/11236; WO 95/15982; WO 95/20401; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727 and 5,733,743, each incorporated herein by reference in their entirety and for all purposes.

As described in the above references, after phage selection, the antibody coding regions from the phage can be isolated and used to generate whole antibodies, including human antibodies, or any other desired antigen binding fragment, and expressed in any desired host including mammalian cells, insect cells, plant cells, yeast, and bacteria. For example, techniques to recombinantly produce Fab, Fab′ and F(ab′)₂ fragments can also be employed using methods known in the art such as those disclosed in WO 92/22324; Mullinax et al., BioTechniques 12: 864-869, 1992; and Sawai et al., AJRI 34: 26-34, 1995; and Better et al., Science 240: 1041-1043, 1988.

Examples of techniques which can be used to produce single-chain Fvs and antibodies include those described in U.S. Pat. Nos. 4,946,778 and 5,258,498, each incorporated herein by reference in their entirety and for all purposes; Huston et al., Methods in Enzymology, 203: 46-88, 1991; Shu, L. et al., PNAS 90: 7995-7999, 1993; and Skerra et al., Science 240: 1038-1040, 1988. For some uses, including in vivo use of antibodies in humans and in vitro detection assays, it can be preferable to use chimeric, humanized, or human antibodies. Methods for producing chimeric antibodies are known in the art. See e.g., Morrison, Science 229: 1202, 1985; Oi et al., BioTechniques 4: 214, 1986; Gillies et al., J. Immunol. Methods, 125: 191-202, 1989; and U.S. Pat. No. 5,807,715. Antibodies can be humanized using a variety of techniques including CDR-grafting (EP 0 239 400; WO 91/09967; U.S. Pat. Nos. 5,530,101; and 5,585,089), veneering or resurfacing (EP 0 592 106; EP 0 519 596; Padlan E. A., Molecular Immunology, 28: 489-498, 1991; Studnicka et al., Protein Engineering 7: 805-814, 1994; Roguska et al., PNAS 91: 969-973, 1994), and chain shuffling (U.S. Pat. No. 5,565,332). Human antibodies can be made by a variety of methods known in the art including phage display methods described above. See also, U.S. Pat. Nos. 4,444,887, 4,716,111, 5,545,806, and 5,814,318; and WO 98/46645, WO 98/50433, WO 98/24893, WO 98/16654, WO 96/34096, WO 96/33735, and WO 91/10741, each incorporated herein by reference in their entirety and for all purposes.

Further included in the present invention are antibodies recombinantly fused or chemically conjugated (including both covalently and non-covalently conjugations) to a polypeptide of the present invention. The antibodies can be specific for antigens other than polypeptides of the present invention. For example, antibodies can be used to target the polypeptides of the present invention to particular cell types, either in vitro or in vivo, by fusing or conjugating the polypeptides of the present invention to antibodies specific for particular cell surface receptors. Antibodies fused or conjugated to the polypeptides of the present invention can also be used in in vitro immunoassays and purification methods using methods known in the art. See e.g., Harbor et al. supra and WO 93/21232; EP 0 439 095; Naramura et al., Immunol. Lett. 39: 91-99, 1994; U.S. Pat. No. 5,474,981, incorporated herein by reference in its entirety and for all purposes; Gillies et al., PNAS 89: 1428-1432, 1992; Fell et al., J. Immunol. 146: 2446-2452, 1991.

sCFV Phage Libraries

One approach for a phage display library is to identify an antibody composition, useful as an antibody-cytotoxin conjugate molecule for treatment of a neoplastic disease that specifically binds to a cell surface receptor on a metastatic cell, for example, an activated integrin receptor. scFv phage-libraries have been used. (see, e.g., Huston et al., Proc. Natl. Acad. Sci. U.S.A., 85: 5879-5883, 1988; Chaudhary et al., Proc. Natl. Acad. Sci. U.S.A., 87: 1066-1070, 1990. Various embodiments of scFv libraries displayed on bacteriophage coat proteins have been described. Refinements of phage display approaches are also known, for example as described in WO96/06213 and WO92/01047 (Medical Research Council et al.) and WO97/08320 (Morphosys), which are incorporated herein by reference. The display of Fab libraries is also known, for instance as described in WO92/01047 (CAT/MRC) and WO91/17271 (Affymax).

Hybrid antibodies or hybrid antibody fragments that are cloned into a display vector can be selected against the appropriate antigen associated with a metastatic cell, e.g., a cell surface receptor or an activated cell surface receptor on a metastatic tumor cell, in order to identify variants that maintained good binding activity, because the antibody or antibody fragment will be present on the surface of the phage or phagemid particle. See for example Barbas III et al., Phage Display, A Laboratory Manual, Cold Spring Harbor Laboratory Press, Cold Spring Harbor, N.Y., 2001, the contents of which are incorporated herein by reference. For example, in the case of Fab fragments, the light chain and heavy chain Fd products are under the control of a lac promoter, and each chain has a leader signal fused to it in order to be directed to the periplasmic space of the bacterial host. It is in this space that the antibody fragments will be able to properly assemble. The heavy chain fragments are expressed as a fusion with a phage coat protein domain which allows the assembled antibody fragment to be incorporated into the coat of a newly made phage or phagemid particle. Generation of new phagemid particles requires the addition of helper phage which contain all the necessary phage genes. Once a library of antibody fragments is presented on the phage or phagemid surface, a process termed panning follows. This is a method whereby i) the antibodies displayed on the surface of phage or phagemid particles are bound to the desired antigen; ii) non-binders are washed away; iii) bound particles are eluted from the antigen; and iv) eluted particles are exposed to fresh bacterial hosts in order to amplify the enriched pool for an additional round of selection. Typically three or four rounds of panning are performed prior to screening antibody clones for specific binding. In this way phage/phagemid particles allow the linkage of binding phenotype (antibody) with the genotype (DNA) making the use of antibody display technology very successful. However, other vector formats could be used for this humanization process, such as cloning the antibody fragment library into a lytic phage vector (modified T7 or Lambda Zap systems) for selection and/or screening.

After selection of desired hybrid antibodies and/or hybrid antibody fragments, it is contemplated that they can be produced in large volume by any technique known to those skilled in the art, e.g., prokaryotic or eukaryotic cell expression and the like. For example, hybrid antibodies or fragments can be produced by using conventional techniques to construct an expression vector that encodes an antibody heavy chain in which the CDRs and, if necessary, a minimal portion of the variable region framework, that are required to retain original species antibody binding specificity (as engineered according to the techniques described herein) are derived from the originating species antibody and the remainder of the antibody is derived from a target species immunoglobulin which can be manipulated as described herein, thereby producing a vector for the expression of a hybrid antibody heavy chain.

In a detailed embodiment, a single-chain Fv (scFv) antibody library can be prepared from the peripheral blood lymphocytes of 5, 10, 15, or 20 or more patients with various cancer diseases. Completely human high-affinity scFv antibodies can then be selected by using synthetic sialyl Lewisx and Lewisx BSA conjugates. In one study, these human scFv antibodies were specific for sialyl Lewisx and Lewisx, as demonstrated by ELISA, BIAcore, and flow cytometry binding to the cell surface of pancreatic adenocarcinoma cells. Nucleotide sequencing revealed that at least four unique scFv genes were obtained. The K_{d} values ranged from 1.1 to 6.2×10⁻⁷ M that were comparable to the affinities of mAbs derived from the secondary immune response. These antibodies could be valuable reagents for probing the structure and function of carbohydrate antigens and in the treatment of human tumor diseases. Mao et al., Proc. Natl. Acad. Sci. U.S.A. 96: 6953-6958, 1999.

In a further detailed embodiment, phage displayed combinatorial antibody libraries can be used to generate and select a wide variety of antibodies to an appropriate antigen associated with a metastatic cell, e.g., a cell surface receptor or an activated cell surface receptor on a metastatic tumor cell. The phage coat proteins pVII and pIX can be used to display the heterodimeric structure of the antibody Fv region. Aspects of this technology have been extended to construct a large, human single-chain Fv (scFv) library of 4.5×10⁹ members displayed on pIX of filamentous bacteriophage. Furthermore, the diversity, quality, and utility of the library were demonstrated by the selection of scFv clones against six different protein antigens. Notably, more than 90% of the selected clones showed positive binding for their respective antigens after as few as three rounds of panning. Analyzed scFvs were also found to be of high affinity. For example, kinetic analysis (BIAcore) revealed that scFvs against staphylococcal enterotoxin B and cholera toxin B subunit had a nanomolar and subnanomolar dissociation constant, respectively, affording affinities comparable to, or exceeding that, of mAbs obtained from immunization. High specificity was also attained, not only between very distinct proteins, but also in the case of more closely related proteins, e.g., Ricinus communes (“ricin”) agglutinins (RCA₆₀ and RCA₁₂₀), despite >80% sequence homology between the two. The results suggested that the performance of pIX-display libraries can potentially exceed that of the pill-display format and make it ideally suited for panning a wide variety of target antigens. Gao et al., Proc. Natl. Acad. Sci. U.S.A. 99: 12612-12616, 2001.

Specific binding between an antibody or other binding agent and an antigen means a binding affinity of at least 10⁻⁶ M. Preferred binding agents bind with affinities of at least about 10⁻⁷ M, and preferably 10⁻⁸ M to 10⁻⁹ M, 10⁻¹⁰ M, 10⁻¹¹ M, or 10⁻¹² M.

Immune Response

“Immune cell response” refers to the response of immune system cells to external or internal stimuli (e.g., antigen, cell surface receptors, activated integrin receptors, cytokines, chemokines, and other cells) producing biochemical changes in the immune cells that result in immune cell migration, killing of target cells, phagocytosis, production of antibodies, other soluble effectors of the immune response, and the like.

“Immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of cancerous cells, metastatic tumor cells, malignant melanoma, invading pathogens, cells or tissues infected with pathogens, or, in cases of autoimmunity or pathological inflammation, normal human cells or tissues.

“Lymphocyte” as used herein has the normal meaning in the art, and refers to any of the mononuclear, nonphagocytic leukocytes, found in the blood, lymph, and lymphoid tissues, e.g., B and T lymphocytes.

“T lymphocyte response” and “T lymphocyte activity” are used here interchangeably to refer to the component of immune response dependent on T lymphocytes (e.g., the proliferation and/or differentiation of T lymphocytes into helper, cytotoxic killer, or suppressor T lymphocytes, the provision of signals by helper T lymphocytes to B lymphocytes that cause or prevent antibody production, the killing of specific target cells by cytotoxic T lymphocytes, and the release of soluble factors such as cytokines that modulate the function of other immune cells).

Components of an immune response can be detected in vitro by various methods that are well known to those of ordinary skill in the art. For example, (1) cytotoxic T lymphocytes can be incubated with radioactively labeled target cells and the lysis of these target cells detected by the release of radioactivity, (2) helper T lymphocytes can be incubated with antigens and antigen presenting cells and the synthesis and secretion of cytokines measured by standard methods (Windhagen A; et al., Immunity, 2: 373-80, 1995), (3) antigen presenting cells can be incubated with whole protein antigen and the presentation of that antigen on MHC detected by either T lymphocyte activation assays or biophysical methods (Harding et al., Proc. Natl. Acad. Sci., 86: 4230-4, 1989), (4) mast cells can be incubated with reagents that cross-link their Fc-epsilon receptors and histamine release measured by enzyme immunoassay (Siraganian et al., TIPS, 4: 432-437, 1983).

Similarly, products of an immune response in either a model organism (e.g., mouse) or a human patient can also be detected by various methods that are well known to those of ordinary skill in the art. For example, (1) the production of antibodies in response to vaccination can be readily detected by standard methods currently used in clinical laboratories, e.g., an ELISA; (2) the migration of immune cells to sites of inflammation can be detected by scratching the surface of skin and placing a sterile container to capture the migrating cells over scratch site (Peters et al., Blood, 72: 1310-5, 1988); (3) the proliferation of peripheral blood mononuclear cells in response to mitogens or mixed lymphocyte reaction can be measured using ³H-thymidine; (4) the phagocytic capacity of granulocytes, macrophages, and other phagocytes in PBMCs can be measured by placing PMBCs in wells together with labeled particles (Peters et al., Blood, 72: 1310-5, 1988); and (5) the differentiation of immune system cells can be measured by labeling PBMCs with antibodies to CD molecules such as CD4 and CD8 and measuring the fraction of the PBMCs expressing these markers.

For convenience, immune responses are often described in the present invention as being either “primary” or “secondary” immune responses. A primary immune response, which is also described as a “protective” immune response, refers to an immune response produced in an individual as a result of some initial exposure (e.g., the initial “immunization”) to a particular antigen, e.g., cell surface receptor, or activated integrin receptor. Such an immunization can occur, for example, as the result of some natural exposure to the antigen (for example, from initial infection by some pathogen that exhibits or presents the antigen) or from antigen presented by cancer cells of some tumor in the individual (for example, malignant melanoma). Alternatively, the immunization can occur as a result of vaccinating the individual with a vaccine containing the antigen. For example, the vaccine can be a cancer vaccine comprising one or more antigens from a cancer cell e.g., malignant melanoma.

A primary immune response can become weakened or attenuated over time and can even disappear or at least become so attenuated that it cannot be detected. Accordingly, the present invention also relates to a “secondary” immune response, which is also described here as a “memory immune response.” The term secondary immune response refers to an immune response elicited in an individual after a primary immune response has already been produced. Thus, a secondary or immune response can be elicited, e.g., to enhance an existing immune response that has become weakened or attenuated, or to recreate a previous immune response that has either disappeared or can no longer be detected. An agent that can be administrated to elicit a secondary immune response is after referred to as a “booster” since the agent can be said to “boost” the primary immune response.

As an example, and not by way of limitation, a secondary immune response can be elicited by re-introducing to the individual an antigen that elicited the primary immune response (for example, by re-administrating a vaccine). However, a secondary immune response to an antigen can also be elicited by administrating other agents that can not contain the actual antigen. For example, the present invention provides methods for potentiating a secondary immune response by administrating an antibody to activated integrin receptor to an individual. In such methods the actual antigen need not necessarily be administered with the antibody to activated integrin receptor and the composition containing the antibody need not necessarily contain the antigen. The secondary or memory immune response can be either a humoral (antibody) response or a cellular response. A secondary or memory humoral response occurs upon stimulation of memory B cells that were generated at the first presentation of the antigen. Delayed type hypersensitivity (DTH) reactions are a type of cellular secondary or memory immune response that are mediated by CD4⁺ cells. A first exposure to an antigen primes the immune system and additional exposure(s) results in a DTH.

“Immunologically cross-reactive” or “immunologically reactive” refers to an antigen which is specifically reactive with an antibody which was generated using the same (“immunologically reactive”) or different (“immunologically cross-reactive”) antigen. Generally, the antigen is activated integrin receptor, or more typically an α₃β₁ integrin receptor or subsequence thereof.

“Immunologically reactive conditions” refers to conditions which allow an antibody, generated to a particular epitope of an antigen, to bind to that epitope to a detectably greater degree than the antibody binds to substantially all other epitopes, generally at least two times above background binding, preferably at least five times above background. Immunologically reactive conditions are dependent upon the format of the antibody binding reaction and typically are those utilized in immunoassay protocols. See, Harlow & Lane, Antibodies, A Laboratory Manual, Cold Spring Harbor Publications, New York, 1988 (Harlow & Lane) for a description of immunoassay formats and conditions.

“Cell surface receptor” refers to molecules and complexes of molecules capable of receiving a signal and the transmission of such a signal across the plasma membrane of a cell. An example of a “cell surface receptor” of the present invention is an activated integrin receptor, for example, an activated α₃β₁ integrin receptor on a metastatic cell.

“Nonspecific T cell activation” refers to the stimulation of T cells independent of their antigenic specificity.

“Effector cell” refers to an immune cell which is involved in the effector phase of an immune response, as opposed to the cognitive and activation phases of an immune response. Exemplary immune cells include a cell of a myeloid or lymphoid origin, e.g., lymphocytes (e.g., B cells and T cells including cytolytic T cells (CTLs)), killer cells, natural killer cells, macrophages, monocytes, eosinophils, neutrophils, polymorphonuclear cells, granulocytes, mast cells, and basophils. Effector cells express specific Fe receptors and carry out specific immune functions. An effector cell can induce antibody-dependent cell-mediated cytotoxicity (ADCC), e.g., a neutrophil capable of inducing ADCC. For example, monocytes, macrophages, neutrophils, eosinophils, and lymphocytes which express FcαR are involved in specific killing of target cells and presenting antigens to other components of the immune system, or binding to cells that present antigens. An effector cell can also phagocytose a target antigen, target cell, metastatic cancer cell, or microorganism.

“Target cell” refers to any undesirable cell in a subject (e.g., a human or animal) that can be targeted by the Ab or Ab composition of the invention. The target cell can be a cell expressing or overexpressing human activated integrin receptor. Cells expressing human activated integrin receptor can include tumor cells, e.g., malignant melanoma.

Targets of interest for antibody compositions metastatic cancer cells, e.g., malignant melanoma, include, but are not limited to, cell surface receptors, growth factor receptors, antibodies, including anti-idiotypic antibodies and autoantibodies present in cancer, such as metastatic cancer and malignant melanoma. Other targets are adhesion proteins such as integrins, selectins, and immunoglobulin superfamily members. Springer, Nature, 346: 425-433, 1990; Osborn, Cell, 62: 3, 1990; Hynes, Cell, 69: 11, 1992. Other targets of interest are growth factor receptors (e.g., FGFR, PDGFR, EGF, her/neu, NGFR, and VEGF) and their ligands. Other targets are G-protein receptors and include substance K receptor, the angiotensin receptor, the α- and β-adrenergic receptors, the serotonin receptors, and PAF receptor. See, e.g., Gilman, Ann. Rev. Biochem. 56: 625-649, 1987. Other targets include ion channels (e.g., calcium, sodium, potassium channels, channel proteins that mediate multidrug resistance), muscarinic receptors, acetylcholine receptors, GABA receptors, glutamate receptors, and dopamine receptors (see Harpold, U.S. Pat. No. 5,401,629 and U.S. Pat. No. 5,436,128). Other targets are cytokines, such as interleukins IL-1 through IL-13, tumor necrosis factors α- and β, interferons α-, β- and γ, tumor growth factor Beta (TGF-β), colony stimulating factor (CSF) and granulocyte monocyte colony stimulating factor (GM-CSF). See Human Cytokines: Handbook for Basic & Clinical Research (Aggrawal et al. eds., Blackwell Scientific, Boston, Mass., 1991). Other targets are hormones, enzymes, and intracellular and intercellular messengers, such as adenyl cyclase, guanyl cyclase, and phospholipase C. Drugs are also targets of interest. Target molecules can be human, mammalian or bacterial. Other targets are antigens, such as proteins, glycoproteins and carbohydrates from microbial pathogens, both viral and bacterial, and tumors. Still other targets are described in U.S. Pat. No. 4,366,241, incorporated herein by reference in its entirety and for all purposes. Some agents screened by the target merely bind to a target. Other agents agonize or antagonize the target.

Cancer and Cancer Treatment

“Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize), as well as any of a number of characteristic structural and/or molecular features. A “cancerous” or “malignant cell” is understood as a cell having specific structural properties, lacking differentiation and being capable of invasion and metastasis. Examples of cancers are, breast, lung, brain, bone, liver, kidney, colon, and prostate cancer. (see DeVita, V. et al. (eds.), 2001, Cancer Principles and Practice of Oncology, 6th. Ed., Lippincott Williams & Wilkins, Philadelphia, Pa.; this reference is herein incorporated by reference in its entirety for all purposes).

“Cancer-associated” refers to the relationship of a nucleic acid and its expression, or lack thereof, or a protein and its level or activity, or lack thereof, to the onset of malignancy in a subject cell. For example, cancer can be associated with expression of a particular gene that is not expressed, or is expressed at a lower level, in a normal healthy cell. Conversely, a cancer-associated gene can be one that is not expressed in a malignant cell (or in a cell undergoing transformation), or is expressed at a lower level in the malignant cell than it is expressed in a normal healthy cell.

In the context of the cancer, the term “transformation” refers to the change that a normal cell undergoes as it becomes malignant. In eukaryotes, the term “transformation” can be used to describe the conversion of normal cells to malignant cells in cell culture.

“Proliferating cells” are those which are actively undergoing cell division and growing exponentially. “Loss of cell proliferation control” refers to the property of cells that have lost the cell cycle controls that normally ensure appropriate restriction of cell division. Cells that have lost such controls proliferate at a faster than normal rate, without stimulatory signals, and do not respond to inhibitory signals.

“Advanced cancer” means cancer that is no longer localized to the primary tumor site, or a cancer that is Stage III or IV according to the American Joint Committee on Cancer (AJCC).

“Well tolerated” refers to the absence of adverse changes in health status that occur as a result of the treatment and would affect treatment decisions.

“Metastatic” or “metastatic state” refers to tumor cells, e.g., human melanoma cells, that are able to establish secondary tumor lesions in the lungs, liver, bone or brain—for example, in immune deficient mice upon injection into the mammary fat pad and/or the circulation of the immune deficient mouse.

“Non-metastatic” or “non-metastatic state” refers to tumor cells, e.g., human melanoma cells that are unable to establish secondary tumor lesions in the lungs, liver, bone or brain or other target organs of melanoma metastasis—for example, in immune deficient mice upon injection into the mammary fat pad and/or the circulation. The human tumor cells used herein and addressed herein as non-metastatic are able to establish primary tumors upon injection into the mammary fat pad of the immune deficient mouse, but they are unable to disseminate from those primary tumors.

“Differentially produced” refers to a compound, e.g., an integrin receptor, produced by a cell that is produced at an altered level in a metastatic cell compared to a non-metastatic cell. The altered level can be lower or higher when comparing metastatic to non-metastatic cells. The altered levels can be detectable and can be the basis for therapeutic treatment of a neoplastic disease in a mammalian subject.

“Differentially produced” refers to both quantitative as well as qualitative differences in the temporal and tissue expression patterns of a gene or a protein. For example, a differentially produced gene can have its expression activated or completely inactivated in normal versus disease conditions. Such a qualitatively regulated gene can exhibit an expression pattern within a given tissue or cell type that is detectable in either control or disease conditions, but is not detectable in both. Differentially produced genes can represent “profile genes,” or “target genes” and the like.

Similarly, a differentially produced protein can have its expression activated or completely inactivated in normal versus disease conditions. Such a qualitatively regulated protein can exhibit an expression pattern within a given tissue or cell type that is detectable in either control or disease conditions, but is not detectable in both. Moreover, differentially produced genes can represent “profile proteins”, “target proteins” and the like.

Methods for treating a neoplastic disease provide for treatment with an antibody-cytotoxin conjugate molecule of the present invention. Blockade of activated integrin receptor by antibody compositions can enhance the memory or secondary immune response to cancerous cells in the patient, thereby facilitating cancer treatment. Antibodies to activated integrin receptor within an antibody-cytotoxin conjugate molecule can be combined with an immunogenic agent, such as cancerous cells, purified tumor antigens (including recombinant proteins, peptides, and carbohydrate molecules), cells, and cells transfected with genes encoding immune stimulating cytokines and cell surface antigens, or used alone, to stimulate immunity.

An antibody to activated integrin receptor when combined in an antibody-cytotoxin conjugate molecule is effective when following a vaccination protocol. Many experimental strategies for vaccination against tumors have been devised (see Rosenberg, S., ASCO Educational Book Spring: 60-62, 2000; Logothetis, C., ASCO Educational Book Spring: 300-302, 2000; Khayat, D., ASCO Educational Book Spring: 414-428, 2000; Foon, K., ASCO Educational Book Spring: 730-738, 2000; see also Restifo, N. et al., Cancer: Principles and Practice of Oncology, 61: 3023-3043, 1997. In one of these strategies, a vaccine is prepared using autologous or allogeneic tumor cells. These cellular vaccines have been shown to be most effective when the tumor cells are transduced to express GM-CSF. GM-CSF has been shown to be a potent activator of antigen presentation for tumor vaccination. Dranoff et al., Proc. Natl. Acad. Sci. U.S.A., 90: 3539-43, 1993.

Antibodies to activated integrin receptor can boost GMCSF-modified tumor cell vaccines, and have improved efficacy of vaccines in a number of experimental tumor models such as mammary carcinoma (Hurwitz et al., 1998, supra), primary prostate cancer (Hurwitz et al., Cancer Research, 60: 2444-8, 2000) and melanoma (van Elsas et al., J. Exp. Med., 190: 355-66, 1999). In these instances, non-immunogenic tumors, such as the B 16 melanoma, have been rendered susceptible to destruction by the immune system. The tumor cell vaccine can also be modified to express other immune activators such as IL2, and costimulatory molecules, among others.

The study of gene expression and large scale gene expression patterns in various tumors has led to the definition of so called “tumor specific antigens” (Rosenberg, Immunity, 10: 281-7, 1999). In many cases, these tumor specific antigens are differentiation antigens expressed in the tumors and in the cell from which the tumor arose, for example melanocyte antigens gp100, MAGE antigens, Trp-2. More importantly, many of these antigens can be shown to be the targets of tumor specific T cells found in the host. Antibodies to activated integrin receptor can be used as a boosting agent in conjunction with vaccines based on recombinant versions of proteins and/or peptides found to be expressed in a tumor in order to potentiate a secondary or memory immune response to these proteins. These proteins are normally viewed by the immune system as self antigens and are therefore tolerant to them. The tumor antigen can also include the protein telomerase, which is required for the synthesis of telomeres of chromosomes and which is expressed in more than 85% of human cancers and in only a limited number of somatic tissues (Kim et al., Science, 266: 2011-2013, 1994). These somatic tissues can be protected from immune attack by various means. Tumor antigen can also be “neo-antigens” expressed in cancer cells because of somatic mutations that alter protein sequence or create fusion proteins between two unrelated sequences (e.g., bcr-abl in the Philadelphia chromosome), or idiotype from B cell tumors. Other tumor vaccines can include the proteins from viruses implicated in human cancers such a Human Papilloma Viruses (HPV), Hepatitis Viruses (HBV and HCV) and Kaposi's Herpes Sarcoma Virus (KHSV). Another form of tumor specific antigen which can be used in conjunction with antibodies to activated integrin receptor is purified heat shock proteins (HSP) isolated from the tumor tissue itself. These heat shock proteins contain fragments of proteins from the tumor cells and these HSPs are highly efficient at delivery to antigen presenting cells for eliciting tumor immunity (Suot et al., Science, 269: 1585-1588, 1995; Tamura et al., Science, 278: 117-120, 1997.

Dendritic cells (DC) are potent antigen presenting cells that can be used to prime antigen-specific responses to activated integrin receptors on metastatic tumor cells. DC's can be produced ex vivo and loaded with various protein and peptide antigens as well as tumor cell extracts (Nestle et al., Nature Medicine, 4: 328-332, 1998). DCs can also be transduced by genetic means to express these tumor antigens as well. DCs have also been fused directly to tumor cells for the purposes of immunization (Kugler et al., Nature Medicine, 6: 332-336, 2000). As a method of vaccination, DC immunization can be effectively boosted with antibodies to activated integrin receptor to activate more potent anti-tumor responses.

Another type of anti-tumor vaccine that can be combined with antibodies to activated integrin receptor is a vaccine prepared from a melanoma cell line lysate, in conjunction with an immunological adjuvant, such as the MELACINE™ vaccine, a mixture of lysates from two human melanoma cell lines plus DETOX™ immunological adjuvant. Vaccine treatment can be boosted with anti-activated integrin receptor antibodies, with or without additional chemotherapeutic treatment.

An antibody-cytotoxin conjugate comprising antibodies to activated integrin receptor can also be used to boost immunity induced through standard cancer treatments. In these instances, it can be possible to reduce the dose of chemotherapeutic reagent administered (Mokyr et al., Cancer Research, 58: 5301-5304, 1998). The scientific rationale behind the combined use of antibodies to activated integrin receptor and chemotherapy is that cell death, a consequence of the cytotoxic action of most chemotherapeutic compounds, should result in increased levels of tumor antigen in the antigen presentation pathway. Thus, antibodies to activated integrin receptor can boost an immune response primed to chemotherapy release of tumor cells. Examples of chemotherapeutic agents combined with treatment with antibodies to activated integrin receptor can include, but are not limited to, Actinomycetes or Streptomyces antibiotics, duocarmycin, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, duocarmycin, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol™), pilocarpine, prochloroperazine, rituximab, saproin, tamoxifen, taxol, topotecan hydrochloride, trastuzumab, vinblastine, vincristine and vinorelbine tartrate. For prostate cancer treatment, a preferred chemotherapeutic agent with which anti-activated integrin receptor can be combined is paclitaxel (Taxol™). For melanoma cancer treatment, a preferred chemotherapeutic agent with which anti-activated integrin receptor can be combined is dacarbazine (DTIC).

Other combination therapies that can result in immune system priming through cell death are radiation, surgery, and hormone deprivation (Kwon et al., Proc. Natl. Acad. Sci. U.S.A., 96: 15074-9, 1999. Each of these protocols creates a source of tumor antigen in the host. For example, any manipulation of the tumor at the time of surgery can greatly increase the number of cancer cells in the blood (Schwartz et al., Principles of Surgery 1984. 4^{th} ed. p. 338).

Angiogenesis inhibitors can also be combined with antibodies to activated integrin receptor. Inhibition of angiogenesis leads to tumor cell death which can feed tumor antigen into host antigen presentation pathways. All of these cause tumor release and possible immune system priming that antibodies to activated integrin receptor can boost.

“Treating” or “treatment” includes the administration of the antibody compositions, antibody-cytotoxin conjugate molecule compounds or agents of the present invention to prevent or delay the onset of the symptoms, complications, or biochemical indicia of a disease, alleviating the symptoms or arresting or inhibiting further development of the disease, condition, or disorder (e.g., cancer or metastatic cancer). “Treating” or “treatment” of cancer or metastatic cancer using the methods of the present invention refers to any indicia of success in the treatment or amelioration or prevention of an cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with neoplastic disease. The term “therapeutic effect” refers to the reduction, elimination, or prevention of the disease, symptoms of the disease, or side effects of the disease in the subject.

“In combination with”, “combination therapy” and “combination products” refer, in certain embodiments, to the concurrent administration to a patient of a first therapeutic and the compounds as used herein. When administered in combination, each component can be administered at the same time or sequentially in any order at different points in time. Thus, each component can be administered separately but sufficiently closely in time so as to provide the desired therapeutic effect.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

Antibody Therapeutics

When used in vivo for therapy, the antibodies of the subject invention are administered to the patient in therapeutically effective amounts (i.e., amounts that have desired therapeutic effect). They will normally be administered parenterally. The dose and dosage regimen will depend upon the degree of the infection, the characteristics of the particular antibody or immunotoxin used, e.g., its therapeutic index, the patient, and the patient's history. Advantageously the antibody or immunotoxin is administered continuously over a period of 1-2 weeks, intravenously to treat cells in the vasculature and subcutaneously and intraperitoneally to treat regional lymph nodes. Optionally, the administration is made during the course of adjunct therapy such as combined cycles of radiation, chemotherapeutic treatment, or administration of tumor necrosis factor, interferon or other cytoprotective or immunomodulatory agent.

For parenteral administration the antibodies will be formulated in a unit dosage injectable form (solution, suspension, emulsion) in association with a pharmaceutically acceptable parenteral vehicle. Such vehicles are inherently nontoxic, and non-therapeutic. Examples of such vehicle are water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability, e.g., buffers and preservatives. The antibodies will typically be formulated in such vehicles at concentrations of about 1 mg/ml to 10 mg/ml.

Use of IgM antibodies can be preferred for certain applications, however IgG molecules by being smaller can be more able than IgM molecules to localize to certain types of infected cells.

There is evidence that complement activation in vivo leads to a variety of biological effects, including the induction of an inflammatory response and the activation of macrophages (Unanue and Benecerraf, Textbook of Immunology, 2nd Edition, Williams & Wilkins, p. 218 (1984)). The increased vasodilation accompanying inflammation can increase the ability of various agents to localize in infected cells. Therefore, antigen-antibody combinations of the type specified by this invention can be used therapeutically in many ways. Additionally, purified antigens (Hakomori, Ann. Rev. Immunol. 2:103, 1984) or anti-idiotypic antibodies (Nepom et al., Proc. Natl. Acad. Sci. USA 81: 2864, 1985; Koprowski et al., Proc. Natl. Acad. Sci. USA 81: 216, 1984) relating to such antigens could be used to induce an active immune response in human patients. Such a response includes the formation of antibodies capable of activating human complement and mediating ADCC and by such mechanisms cause infected cell destruction.

Optionally, the antibodies of this invention are useful as antibody-cytotoxin conjugate molecules, as exemplified by the administration for treatment of neoplastic disease.

The antibody compositions used in therapy are formulated and dosages established in a fashion consistent with good medical practice taking into account the disorder to be treated, the condition of the individual patient, the site of delivery of the composition, the method of administration and other factors known to practitioners. The antibody compositions are prepared for administration according to the description of preparation of polypeptides for administration, infra.

As is well understood in the art, biospecific capture reagents include antibodies, binding fragments of antibodies which bind to activated integrin receptors on metastatic cells (e.g., single chain antibodies, Fab′ fragments, F(ab)′₂ fragments, and scFv proteins and affibodies (Affibody, Teknikringen 30, floor 6, Box 700 04, Stockholm SE-10044, Sweden; See U.S. Pat. No. 5,831,012, incorporated herein by reference in its entirety and for all purposes)). Depending on intended use, they also can include receptors and other proteins that specifically bind another biomolecule.

The hybrid antibodies and hybrid antibody fragments include complete antibody molecules having full length heavy and light chains, or any fragment thereof, such as Fab, Fab′, F(ab′)₂, Fd, scFv, antibody light chains and antibody heavy chains. Chimeric antibodies which have variable regions as described herein and constant regions from various species are also suitable. See for example, U.S. Application No. 20030022244.

Initially, a predetermined target object is chosen to which an antibody can be raised. Techniques for generating monoclonal antibodies directed to target objects are well known to those skilled in the art. Examples of such techniques include, but are not limited to, those involving display libraries, xeno or humab mice, hybridomas, and the like. Target objects include any substance which is capable of exhibiting antigenicity and are usually proteins or protein polysaccharides. Examples include receptors, enzymes, hormones, growth factors, peptides and the like. It should be understood that not only are naturally occurring antibodies suitable for use in accordance with the present disclosure, but engineered antibodies and antibody fragments which are directed to a predetermined object are also suitable.

Antibodies (Abs) that can be subjected to the techniques set forth herein include monoclonal and polyclonal Abs, and antibody fragments such as Fab, Fab′, F(ab′)₂, Fd, scFv, diabodies, antibody light chains, antibody heavy chains and/or antibody fragments derived from phage or phagemid display technologies. To begin with, an initial antibody is obtained from an originating species. More particularly, the nucleic acid or amino acid sequence of the variable portion of the light chain, heavy chain or both, of an originating species antibody having specificity for a target antigen is needed. The originating species is any species which was used to generate the antibodies or antibody libraries, e.g., rat, mice, rabbit, chicken, monkey, human, and the like. Techniques for generating and cloning monoclonal antibodies are well known to those skilled in the art. After a desired antibody is obtained, the variable regions (V_{H} and V_{L}) are separated into component parts (i.e., frameworks (FRs) and CDRs) using any possible definition of CDRs (e.g., Kabat alone, Chothia alone, Kabat and Chothia combined, and any others known to those skilled in the art). Once that has been obtained, the selection of appropriate target species frameworks is necessary. One embodiment involves alignment of each individual framework region from the originating species antibody sequence with variable amino acid sequences or gene sequences from the target species.

The term “diabodies” refers to small antibody fragments with two antigen-binding sites, which fragments comprise a heavy-chain variable domain (VH) connected to a light-chain variable domain (VL) in the same polypeptide chain (VH VL). By using a linker that is too short to allow pairing between the two domains on the same chain, the domains are forced to pair with the complementary domains of another chain and create two antigen binding sites. Diabodies are described more fully in, for example, EP 404,097; WO 93/11161; and 30 Hollinger et al., Proc. Natl. Acad. Sci. USA, 90:6444-6448 (1993).

After selecting suitable frame work region candidates from the same family and/or the same family member, either or both the heavy and light chain variable regions are produced by grafting the CDRs from the originating species into the hybrid framework regions. Assembly of hybrid antibodies or hybrid antibody fragments having hybrid variable chain regions with regard to either of the above aspects can be accomplished using conventional methods known to those skilled in the art. For example, DNA sequences encoding the hybrid variable domains described herein (i.e., frameworks based on the target species and CDRs from the originating species) can be produced by oligonucleotide synthesis and/or PCR. The nucleic acid encoding CDR regions can also be isolated from the originating species antibodies using suitable restriction enzymes and ligated into the target species framework by ligating with suitable ligation enzymes. Alternatively, the framework regions of the variable chains of the originating species antibody can be changed by site-directed mutagenesis.

Since the hybrids are constructed from choices among multiple candidates corresponding to each framework region, there exist many combinations of sequences which are amenable to construction in accordance with the principles described herein. Accordingly, libraries of hybrids can be assembled having members with different combinations of individual framework regions. Such libraries can be electronic database collections of sequences or physical collections of hybrids.

Assembly of a physical antibody or antibody fragment library is preferably accomplished using synthetic oligonucleotides. In one example, oligonucleotides are designed to have overlapping regions so that they could anneal and be filled in by a polymerase, such as with polymerase chain reaction (PCR). Multiple steps of overlap extension are performed in order to generate the V_{L} and V_{H} gene inserts. Those fragments are designed with regions of overlap with human constant domains so that they could be fused by overlap extension to produce full length light chains and Fd heavy chain fragments. The light and heavy Fd chain regions can be linked together by overlap extension to create a single Fab library insert to be cloned into a display vector. Alternative methods for the assembly of the humanized library genes can also be used. For example, the library can be assembled from overlapping oligonucleotides using a Ligase Chain Reaction (LCR) approach. Chalmers et al., Biotechniques, 30-2: 249-252, 2001.

Various forms of antibody fragments can be generated and cloned into an appropriate vector to create a hybrid antibody library or hybrid antibody fragment library. For example variable genes can be cloned into a vector that contains, in-frame, the remaining portion of the necessary constant domain. Examples of additional fragments that can be cloned include whole light chains, the Fd portion of heavy chains, or fragments that contain both light chain and heavy chain Fd coding sequence. Alternatively, the antibody fragments used for humanization can be single chain antibodies (scFv).

Any selection display system can be used in conjunction with a library according to the present disclosure. Selection protocols for isolating desired members of large libraries are known in the art, as typified by phage display techniques. Such systems, in which diverse peptide sequences are displayed on the surface of filamentous bacteriophage have proven useful for creating libraries of antibody fragments (and the nucleotide sequences that encode them) for the in vitro selection and amplification of specific antibody fragments that bind a target antigen. Scott et al., Science, 249: 386, 1990. The nucleotide sequences encoding the VH and VL regions are linked to gene fragments which encode leader signals that direct them to the periplasmic space of E. coli and as a result the resultant antibody fragments are displayed on the surface of the bacteriophage, typically as fusions to bacteriophage coat proteins (e.g., pIII or pVIII). Alternatively, antibody fragments are displayed externally on lambda phage or T7 capsids (phagebodies). An advantage of phage-based display systems is that, because they are biological systems, selected library members can be amplified simply by growing the phage containing the selected library member in bacterial cells. Furthermore, since the nucleotide sequence that encode the polypeptide library member is contained on a phage or phagemid vector, sequencing, expression and subsequent genetic manipulation is relatively straightforward. Methods for the construction of bacteriophage antibody display libraries and lambda phage expression libraries are well known in the art. McCafferty et al., Nature, 348: 552, 1990; Kang et al., Proc. Natl. Acad. Sci. U.S.A., 88:4363, 1991.

Nucleic Acids and Polypeptides

The terms “identical” or percent “identity”, in the context of two or more nucleic acids or polypeptide sequences, refers to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region (e.g., nucleotide sequence encoding an antibody described herein or amino acid sequence of an antibody described herein), when compared and aligned for maximum correspondence over a comparison window or designated region) as measured using a BLAST or BLAST 2.0 sequence comparison algorithms with default parameters described below, or by manual alignment and visual inspection (see, e.g., NCBI web site). Such sequences are then said to be “substantially identical.” This term also refers to, or can be applied to, the compliment of a test sequence. The term also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math., 1981, 2:482, by the homology alignment algorithm of Needleman & Wunsch, J. Mol. Biol., 1970, 48:443, by the search for similarity method of Pearson & Lipman, Proc. Nat'l. Acad. Sci. USA, 1988, 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology, Ausubel et al., eds. 1995 supplement)).

Programs for searching for alignments are well known in the art, e.g., BLAST and the like. For example, if the target species is human, a source of such amino acid sequences or gene sequences (germline or rearranged antibody sequences) can be found in any suitable reference database such as Genbank, the NCBI protein databank (http://ncbi.nlm.nih.gov/BLAST/), VBASE, a database of human antibody genes (http://www.mrc-cpe.cam.ac.uk/imt-doc), and the Kabat database of immunoglobulins (http://www.immuno.bme.nwu.edu) or translated products thereof. If the alignments are done based on the nucleotide sequences, then the selected genes should be analyzed to determine which genes of that subset have the closest amino acid homology to the originating species antibody. It is contemplated that amino acid sequences or gene sequences which approach a higher degree homology as compared to other sequences in the database can be utilized and manipulated in accordance with the procedures described herein. Moreover, amino acid sequences or genes which have lesser homology can be utilized when they encode products which, when manipulated and selected in accordance with the procedures described herein, exhibit specificity for the predetermined target antigen. In certain embodiments, an acceptable range of homology is greater than about 50%. It should be understood that target species can be other than human.

A preferred example of algorithm that is suitable for determining percent sequence identity and sequence similarity are the BLAST and BLAST 2.0 algorithms, which are described in Altschul et al., Nuc. Acids Res., 1977, 25:3389-3402 and Altschul et al., J. Mol. Biol., 1990, 215:403-410, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 1989, 89:10915) alignments (B) of 50, expectation (E) of 10, M=5, N=−4, and a comparison of both strands.

The terms “polypeptide”, “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids can be referred to herein by either their commonly known three letter symbols or by the one-letter symbols recommended by the IUPAC-IUB Biochemical Nomenclature Commission. Nucleotides, likewise, can be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations”, which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains, e.g., enzymatic domains, extracellular domains, transmembrane domains, pore domains, and cytoplasmic tail domains. Domains are portions of a polypeptide that form a compact unit of the polypeptide and are typically 15 to 350 amino acids long. Exemplary domains include domains with enzymatic activity, e.g., a kinase domain. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A particular nucleic acid sequence also implicitly encompasses “splice variants.” Similarly, a particular protein encoded by a nucleic acid implicitly encompasses any protein encoded by a splice variant of that nucleic acid. “Splice variants”, as the name suggests, are products of alternative splicing of a gene. After transcription, an initial nucleic acid transcript can be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The phrase “stringent hybridization conditions” refers to conditions under which a probe will hybridize to its target subsequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions can also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., Ausubel et al, supra.

For PCR, a temperature of about 36° C. is typical for low stringency amplification, although annealing temperatures can vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min. Protocols and guidelines for low and high stringency amplification reactions are provided, e.g., in Innis et al. PCR Protocols, A Guide to Methods and Applications, Academic Press, Inc. N.Y. (1990).

Fusion Proteins

Antibodies to activated integrin receptor can be used to generate fusion proteins. For example, the antibodies of the present invention, when fused to a second protein, can be used as an antigenic tag. Antibodies raised against activated integrin receptor can be used to indirectly detect the second protein by binding to the polypeptide. Moreover, because secreted proteins target cellular locations based on trafficking signals, the integrin receptor can be used as a targeting molecule once fused to other proteins.

Examples of domains that can be fused to polypeptides include not only heterologous signal sequences, but also other heterologous functional regions. The fusion does not necessarily need to be direct, but can occur through linker sequences.

Moreover, fusion proteins can also be engineered to improve characteristics of the polypeptide. For instance, a region of additional amino acids, particularly charged amino acids, can be added to the N-terminus of the polypeptide to improve stability and persistence during purification from the host cell or subsequent handling and storage. Also, peptide moieties can be added to the polypeptide to facilitate purification. Such regions can be removed prior to final preparation of the polypeptide. The addition of peptide moieties to facilitate handling of polypeptides are familiar and routine techniques in the art.

Moreover, antibody compositions or cell surface receptors, or integrin receptors, including fragments, and specifically epitopes, can be combined with parts of the constant domain of immunoglobulins (IgG), resulting in chimeric polypeptides. These fusion proteins facilitate purification and show an increased half-life in vivo. One reported example describes chimeric proteins consisting of the first two domains of the human CD4-polypeptide and various domains of the constant regions of the heavy or light chains of mammalian immunoglobulins. EP A 394,827; Traunecker et al., Nature, 331: 84-86, 1988. Fusion proteins having disulfide-linked dimeric structures (due to the IgG) can also be more efficient in binding and neutralizing other molecules, than the monomeric secreted protein or protein fragment alone. Fountoulakis et al., J. Biochem. 270: 3958-3964, 1995.

Similarly, EP-A-O 464 533 (Canadian counterpart 2045869) discloses fusion proteins comprising various portions of constant region of immunoglobulin molecules together with another human protein or part thereof. In many cases, the Fc part in a fusion protein is beneficial in therapy and diagnosis, and thus can result in, for example, improved pharmacokinetic properties. (EP-A 0232 262.) Alternatively, deleting the Fc part after the fusion protein has been expressed, detected, and purified, would be desired. For example, the Fc portion can hinder therapy and diagnosis if the fusion protein is used as an antigen for immunizations. In drug discovery, for example, human proteins, such as hIL-5, have been fused with Fc portions for the purpose of high-throughput screening assays to identify antagonists of hIL-5. Bennett et al., J. Molecular Recognition 8: 52-58, 1995; K. Johanson et al., J. Biol. Chem., 270: 9459-9471 1995.

Moreover, the polypeptides can be fused to marker sequences, such as a peptide which facilitates purification of the fused polypeptide. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86: 821-824, 1989, for instance, hexa-histidine provides for convenient purification of the fusion protein. Another peptide tag useful for purification, the “HA” tag, corresponds to an epitope derived from the influenza hemagglutinin protein. Wilson et al., Cell 37: 767, 1984.

Thus, any of these above fusions can be engineered using the polynucleotides or the polypeptides of the present invention.

Expression of Recombinant Antibodies

Chimeric, humanized and human antibodies to cell surface receptor, e.g., activated integrin receptor on metastatic cells, are typically produced by recombinant expression. Recombinant polynucleotide constructs typically include an expression control sequence operably linked to the coding sequences of antibody chains, including naturally-associated or heterologous promoter regions. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the crossreacting antibodies. See U.S. Application No. 20020199213 incorporated herein by reference in its entirety and for all purposes.

These expression vectors are typically replicable in the host organisms either as episomes or as an integral part of the host chromosomal DNA. Commonly, expression vectors contain selection markers, e.g., ampicillin-resistance or hygromycin-resistance, to permit detection of those cells transformed with the desired DNA sequences.

E. coli is one prokaryotic host particularly useful for cloning the DNA sequences of the present invention. Microbes, such as yeast are also useful for expression. Saccharomyces is a preferred yeast host, with suitable vectors having expression control sequences, an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

Mammalian cells are a preferred host for expressing nucleotide segments encoding immunoglobulins or fragments thereof. See Winnacker, From Genes To Clones, (VCH Publishers, NY, 1987). A number of suitable host cell lines capable of secreting intact heterologous proteins have been developed in the art, and include Chinese hamster ovary (CHO) cell lines, various COS cell lines, HeLa cells, L cells and myeloma cell lines. Preferably, the cells are nonhuman. Expression vectors for these cells can include expression control sequences, such as an origin of replication, a promoter, an enhancer, and necessary processing information sites, such as ribosome binding sites, RNA splice sites, polyadenylation sites, and transcriptional terminator sequences. Queen et al., Immunol. Rev. 89: 49, 1986. Preferred expression control sequences are promoters derived from endogenous genes, cytomegalovirus, SV40, adenovirus, bovine papillomavirus, and the like. Co et al., J Immunol. 148: 1149, 1992.

Alternatively, antibody coding sequences can be incorporated in transgenes for introduction into the genome of a transgenic animal and subsequent expression in the milk of the transgenic animal. See, e.g., U.S. Pat. Nos. 5,741,957, 5,304,489, and 5,849,992, each incorporated herein by reference in their entirety and for all purposes. Suitable transgenes include coding sequences for light and/or heavy chains in operable linkage with a promoter and enhancer from a mammary gland specific gene, such as casein or beta lactoglobulin.

The vectors containing the DNA segments of interest can be transferred into the host cell by well-known methods, depending on the type of cellular host. For example, calcium chloride transfection is commonly utilized for prokaryotic cells, whereas calcium phosphate treatment, electroporation, lipofection, biolistics or viral-based transfection can be used for other cellular hosts. Other methods used to transform mammalian cells include the use of polybrene, protoplast fusion, liposomes, electroporation, and microinjection (see generally, Sambrook et al., Molecular Cloning). For production of transgenic animals, transgenes can be microinjected into fertilized oocytes, or can be incorporated into the genome of embryonic stem cells, and the nuclei of such cells transferred into enucleated oocytes.

Once expressed, collections of antibodies are purified from culture media and host cells. Antibodies can be purified according to standard procedures of the art, including HPLC purification, column chromatography, gel electrophoresis and the like. Usually, antibody chains are expressed with signal sequences and are thus released to the culture media. However, if antibody chains are not naturally secreted by host cells, the antibody chains can be released by treatment with mild detergent. Antibody chains can then be purified by conventional methods including ammonium sulfate precipitation, affinity chromatography to immobilized target, column chromatography, gel electrophoresis and the like (see generally Scopes, Protein Purification (Springer-Verlag, N.Y., 1982)).

The above methods result in libraries of nucleic acid sequences encoding antibody chains having specific affinity for a chosen target. The libraries of nucleic acids typically have at least 5, 10, 20, 50, 100, 1000, 10⁴, 10⁵, 10⁶, 10⁷, 10⁸, or 10⁹ different members. Usually, no single member constitutes more than 25 or 50% of the total sequences in the library. Typically, at least 25, 50%, 75, 90, 95, 99 or 99.9% of library members encode antibody chains with specific affinity for the target molecules. In the case of double chain antibody libraries, a pair of nucleic acid segments encoding heavy and light chains respectively is considered a library member. The nucleic acid libraries can exist in free form, as components of any vector or transfected as a component of a vector into host cells.

The nucleic acid libraries can be expressed to generate polyclonal libraries of antibodies having specific affinity for a target. The composition of such libraries is determined from the composition of the nucleotide libraries. Thus, such libraries typically have at least 5, 10, 20, 50, 100, 1000, 10⁴, 10⁵, 10⁶, 10⁷, 10⁸, or 10⁹ members with different amino acid composition. Usually, no single member constitutes more than 25 or 50% of the total polypeptides in the library. The percentage of antibody chains in an antibody chain library having specific affinity for a target is typically lower than the percentage of corresponding nucleic acids encoding the antibody chains. The difference is due to the fact that not all polypeptides fold into a structure appropriate for binding despite having the appropriate primary amino acid sequence to support appropriate folding. In some libraries, at least 25, 50, 75, 90, 95, 99 or 99.9% of antibody chains have specific affinity for the target molecules. Again, in libraries of multi-chain antibodies, each antibody (such as a Fab or intact antibody) is considered a library member. The different antibody chains differ from each other in terms of fine binding specificity and affinity for the target. Some such libraries comprise members binding to different epitopes on the same antigen. Some such libraries comprises at least two members that bind to the same antigen without competing with each other.

Polyclonal libraries of human antibodies resulting from the above methods are distinguished from natural populations of human antibodies both by the high percentages of high affinity binders in the present libraries, and in that the present libraries typically do not show the same diversity of antibodies present in natural populations. The reduced diversity in the present libraries is due to the nonhuman transgenic animals that provide the source materials not including all human immunoglobulin genes. For example, some polyclonal antibody libraries are free of antibodies having lambda light chains. Some polyclonal antibody libraries of the invention have antibody heavy chains encoded by fewer than 10, 20, 30 or 40 V_{H} genes. Some polyclonal antibody libraries of the invention have antibody light chains encoded by fewer than 10, 20, 30 or 40 V_{L} genes.

Modified Antibodies

Also included in the invention are modified antibodies to cell surface receptors, e.g., activated integrin receptors, on metastatic cells.

“Modified antibody” refers to antibodies, such as monoclonal antibodies, chimeric antibodies, and humanized antibodies which have been modified by, e.g., deleting, adding, or substituting portions of the antibody. For example, an antibody can be modified by deleting the constant region and replacing it with a constant region meant to increase half-life, e.g., serum half-life, stability or affinity of the antibody.

The antibody conjugates of the invention can be used to modify a given biological response or create a biological response (e.g., to recruit effector cells). The drug moiety is not to be construed as limited to classical chemical therapeutic agents. For example, the drug moiety can be a protein or polypeptide possessing a desired biological activity. Such proteins can include, for example, an enzymatically active toxin, or active fragment thereof, such as abrin, ricin A, pseudomonas exotoxin, or diphtheria toxin; a protein such as tumor necrosis factor or interferon-alpha; or, biological response modifiers such as, for example, lymphokines, interleukin-1 (“IL-1”), interleukin-2 (“IL-2”), interleukin-6 (“IL-6”), granulocyte macrophage colony stimulating factor (“GM-CSF”), granulocyte colony stimulating factor (“G-CSF”), or other growth factors.

In certain preferred embodiments of the invention, the antibodies and antibody compositions of the invention, for example, can be coupled or conjugated to one or more therapeutic or cytotoxic moieties. As used herein, “cytotoxic moiety” simply means a moiety that inhibits cell growth or promotes cell death when proximate to or absorbed by a cell. Suitable cytotoxic moieties in this regard include radioactive agents or isotopes (radionuclides), chemotoxic agents such as differentiation inducers, inhibitors and small chemotoxic drugs, toxin proteins and derivatives thereof, as well as nucleotide sequences (or their antisense sequence). Therefore, the cytotoxic moiety can be, by way of non-limiting example, a chemotherapeutic agent, a photoactivated toxin or a radioactive agent.

In general, therapeutic agents can be conjugated to the antibodies and antibody compositions of the invention, for example, by any suitable technique, with appropriate consideration of the need for pharmokinetic stability and reduced overall toxicity to the patient. A therapeutic agent can be coupled to a suitable antibody moiety either directly or indirectly (e.g., via a linker group). A direct reaction between an agent and an antibody is possible when each possesses a functional group capable of reacting with the other. For example, a nucleophilic group, such as an amino or sulfhydryl group, can be capable of reacting with a carbonyl-containing group, such as an anhydride or an acid halide, or with an alkyl group containing a good leaving group (e.g., a halide). Alternatively, a suitable chemical linker group can be used. A linker group can function as a spacer to distance an antibody from an agent in order to avoid interference with binding capabilities. A linker group can also serve to increase the chemical reactivity of a substituent on a moiety or an antibody, and thus increase the coupling efficiency. An increase in chemical reactivity can also facilitate the use of moieties, or functional groups on moieties, which otherwise would not be possible.

Suitable linkage chemistries include maleimidyl linkers and alkyl halide linkers (which react with a sulfhydryl on the antibody moiety) and succinimidyl linkers (which react with a primary amine on the antibody moiety). Several primary amine and sulfhydryl groups are present on immunoglobulins, and additional groups can be designed into recombinant immunoglobulin molecules. It will be evident to those skilled in the art that a variety of bifunctional or polyfunctional reagents, both homo- and hetero-functional (such as those described in the catalog of the Pierce Chemical Co., Rockford, Ill.), can be employed as a linker group. Coupling can be affected, for example, through amino groups, carboxyl groups, sulfhydryl groups or oxidized carbohydrate residues (see, e.g., U.S. Pat. No. 4,671,958).

As an alternative coupling method, cytotoxic agents can be coupled to the antibodies and antibody compositions of the invention, for example, through an oxidized carbohydrate group at a glycosylation site, as described in U.S. Pat. Nos. 5,057,313 and 5,156,840. Yet another alternative method of coupling the antibody and antibody compositions to the cytotoxic or imaging moiety is by the use of a non-covalent binding pair, such as streptavidin/biotin, or avidin/biotin. In these embodiments, one member of the pair is covalently coupled to the antibody moiety and the other member of the binding pair is covalently coupled to the cytotoxic or imaging moiety.

Where a cytotoxic moiety is more potent when free from the antibody portion of the immunoconjugates of the present invention, it can be desirable to use a linker group which is cleavable during or upon internalization into a cell, or which is gradually cleavable over time in the extracellular environment. A number of different cleavable linker groups have been described. The mechanisms for the intracellular release of a cytotoxic moiety agent from these linker groups include cleavage by reduction of a disulfide bond (e.g., U.S. Pat. No. 4,489,710), by irradiation of a photolabile bond (e.g., U.S. Pat. No. 4,625,014), by hydrolysis of derivatized amino acid side chains (e.g., U.S. Pat. No. 4,638,045), by serum complement-mediated hydrolysis (e.g., U.S. Pat. No. 4,671,958), and acid-catalyzed hydrolysis (e.g., U.S. Pat. No. 4,569,789).

It can be desirable to couple more than one therapeutic, cytotoxic and/or imaging moiety to an antibody or antibody composition of the invention. By poly-derivatizing the antibodies of the invention, several cytotoxic strategies can be simultaneously implemented, an antibody can be made useful as a contrasting agent for several visualization techniques, or a therapeutic antibody can be labeled for tracking by a visualization technique. In one embodiment, multiple molecules of a cytotoxic moiety are coupled to one antibody molecule. In another embodiment, more than one type of moiety can be coupled to one antibody. For instance, a therapeutic moiety, such as an polynucleotide or antisense sequence, can be conjugated to an antibody in conjunction with a chemotoxic or radiotoxic moiety, to increase the effectiveness of the chemo- or radiotoxic therapy, as well as lowering the required dosage necessary to obtain the desired therapeutic effect. Regardless of the particular embodiment, immunoconjugates with more than one moiety can be prepared in a variety of ways. For example, more than one moiety can be coupled directly to an antibody molecule, or linkers that provide multiple sites for attachment (e.g., dendrimers) can be used. Alternatively, a carrier with the capacity to hold more than one cytotoxic moiety can be used.

As explained above, a carrier can bear the agents in a variety of ways, including covalent bonding either directly or via a linker group, and non-covalent associations. Suitable covalent-bond carriers include proteins such as albumins (e.g., U.S. Pat. No. 4,507,234), peptides, and polysaccharides such as aminodextran (e.g., U.S. Pat. No. 4,699,784), each of which have multiple sites for the attachment of moieties. A carrier can also bear an agent by non-covalent associations, such as non-covalent bonding or by encapsulation, such as within a liposome vesicle (e.g., U.S. Pat. Nos. 4,429,008 and 4,873,088). Encapsulation carriers are especially useful in chemotoxic therapeutic embodiments, as they can allow the therapeutic compositions to gradually release a chemotoxic moiety over time while concentrating it in the vicinity of the target cells.

Preferred radionuclides for use as cytotoxic moieties are radionuclides which are suitable for pharmacological administration. Such radionuclides include ¹²³I, ¹²⁵I, ¹³¹I, ⁹⁰Y, ²¹¹At, ⁶⁷Cu, ¹⁸⁶Re, ¹⁸⁸Re, ²¹²Pb, and ²¹²Bi. Iodine and astatine isotopes are more preferred radionuclides for use in the therapeutic compositions of the present invention, as a large body of literature has been accumulated regarding their use. ¹³¹I is particularly preferred, as are other .beta.-radiation emitting nuclides, which have an effective range of several millimeters. ¹²³I, ¹²⁵I, ¹³¹I, or ²¹¹At can be conjugated to antibody moieties for use in the compositions and methods utilizing any of several known conjugation reagents, including Iodogen, N-succinimidyl 3-[²¹¹At]astatobenzoate, N-succinimidyl 3-[¹³¹I]iodobenzoate (SIB), and, N-succinimidyl 5-[¹³¹I]iodob-3-pyridinecarboxylate (SIPC). Any iodine isotope can be utilized in the recited iodo-reagents. Other radionuclides can be conjugated to the antibody or antibody compositions of the invention by suitable chelation agents known to those of skill in the nuclear medicine arts.

Preferred chemotoxic agents include small-molecule drugs such as methotrexate, and pyrimidine and purine analogs. Preferred chemotoxin differentiation inducers include phorbol esters and butyric acid. Chemotoxic moieties can be directly conjugated to the antibody or antibody compositions of the invention via a chemical linker, or can encapsulated in a carrier, which is in turn coupled to the antibody or antibody compositions of the invention.

Preferred toxin proteins for use as cytotoxic moieties include Actinomycetes or Streptomyces antibiotics, such as duocarmycin. Preferred toxin proteins for use as cytotoxic moieties further include ricin, abrin, diphtheria toxin, cholera toxin, gelonin, Pseudomonas exotoxin, Shigella toxin, pokeweed antiviral protein, and other toxin proteins known in the medicinal biochemistry arts. As these toxin agents can elicit undesirable immune responses in the patient, especially if injected intravascularly, it is preferred that they be encapsulated in a carrier for coupling to the antibody and antibody compositions of the invention.

The cytotoxic moiety of the immunotoxin can be a cytotoxic drug or an enzymatically active toxin of bacterial or plant origin, or an enzymatically active fragment (“A chain”) of such a toxin. Enzymatically active toxins and fragments thereof used are diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolacca americana proteins (PAPI, PAPII, and PAP-S), momordica charantia inhibitor, curcin, crotin, sapaonaria officinalis inhibitor, gelonin, mitogellin, restrictocin, phenomycin, and enomycin. In another embodiment, the antibodies are conjugated to small molecule anticancer drugs. Conjugates of the monoclonal antibody and such cytotoxic moieties are made using a variety of bifunctional protein coupling agents. Examples of such reagents are SPDP, IT, bifunctional derivatives of imidoesters such a dimethyl adipimidate HCl, active esters such as disuccinimidyl suberate, aldehydes such as glutaraldehyde, bis-azido compounds such as bis (p-azidobenzoyl) hexanediamine, bis-diazonium derivatives such as bis-(p-diazoniumbenzoyl)-ethylenediamine, diisocyanates such as tolylene 2,6-diisocyanate, and bis-active fluorine compounds such as 1,5-difluoro-2,4-dinitrobenzene. The lysing portion of a toxin can be joined to the Fab fragment of antibodies.

Advantageously, the antibodies and antibody compositions of the invention specifically binding the external domain of the target receptor, e.g., the activated α₃β₁ integrin receptor, can be conjugated to ricin A chain. Most advantageously the ricin A chain is deglycosylated and produced through recombinant means. An advantageous method of making the ricin immunotoxin is described in Vitetta et al., Science 238: 1098 (1987) which is incorporated by reference in its entirety.

The term “contacted” when applied to a cell is used herein to describe the process by which an antibody, antibody composition, cytotoxic agent or moiety, gene, protein and/or antisense sequence, is delivered to a target cell or is placed in direct proximity with the target cell. This delivery can be in vitro or in vivo and can involve the use of a recombinant vector system.

In another aspect, the present invention features an antibody or antibody composition of the invention, or a fragment thereof, conjugated to a therapeutic moiety, such as a cytotoxin, a drug (e.g., an immunosuppressant) or a radiotoxin. Such conjugates are referred to herein as “immunoconjugates”. Immunoconjugates which include one or more cytotoxins are referred to as “immunotoxins.” A cytotoxin or cytotoxic agent includes any agent that is detrimental to (e.g., kills) cells. Examples include Actinomycetes or Streptomyces antibiotics, duocarmycin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin didne, mitoxantrone, mithramycin, actinomycin D, 1-dehydrotestosterone, glucocorticoids, procaine, tetracaine, lidocaine, propranolol, and puromycin and analogs or homologs thereof.

Suitable therapeutic agents for forming immunoconjugates of the invention include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). In a preferred embodiment, the therapeutic agent is a cytotoxic agent or a radiotoxic agent. In another embodiment, the therapeutic agent is an immunosuppressant. In yet another embodiment, the therapeutic agent is GM-CSF. In a preferred embodiment, the therapeutic agent is doxorubicin (adriamycin), cisplatin bleomycin sulfate, carmustine, chlorambucil, cyclophosphamide hydroxyurea or ricin A.

Antibodies and antibody compositions of the invention also can be conjugated to a radiotoxin, e.g., radioactive iodine, to generate cytotoxic radiopharmaceuticals for treating, for example, a cancer.

Techniques for conjugating such therapeutic moiety to antibodies are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., “The Preparation And Cytotoxic Properties Of Antibody-Toxin Conjugates”, Immunol. Rev., 62: 119-58 (1982).

Uses of Polypeptides or Antibody Compositions

Each of the polypeptides or antibody compositions, e.g., antibodies to cell surface receptors, antibody cytotoxin conjugates, cell surface receptors, such as, activated integrin receptor on a metastatic cell, identified herein can be used in numerous ways. The following description should be considered exemplary and utilizes known techniques.

A polypeptide or antibody composition of the present invention can be used to assay protein levels in a biological sample using antibody-based techniques. For example, protein expression in tissues can be studied with classical immunohistological methods. Jalkanen, M. et al., J. Cell. Biol. 101: 976-985, 1985; Jalkanen, M. et al., J. Cell. Biol. 105: 3087-3096, 1987. Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes or other radioactive agent, such as iodine (¹²⁵I, ¹²¹I), carbon (¹⁴C), sulfur (³⁵S), tritium (³H), indium (¹¹²In), and technetium (⁹⁹mTc), and fluorescent labels, such as fluorescein and rhodamine, and biotin.

In addition to assaying secreted protein levels in a biological sample, proteins or antibody compositions can also be detected in vivo by imaging. Antibody labels or markers for in vivo imaging of protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which can be incorporated into the antibody by labeling of nutrients for the relevant scFv clone.

A protein-specific antibody or antibody fragment which has been labeled with an appropriate detectable imaging moiety, such as a radioisotope (for example, ¹³¹I, ¹¹²In, ⁹⁹mTc), a radio-opaque substance, or a material detectable by nuclear magnetic resonance, is introduced (for example, parenterally, subcutaneously, or intraperitoneally) into the mammal. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹ mTc. The labeled antibody or antibody fragment will then preferentially accumulate at the location of cells which contain the specific protein. In vivo tumor imaging is described in S. W. Burchiel et al., Tumor Imaging: The Radiochemical Detection of Cancer 13, 1982.

Thus, the invention provides a diagnostic method of a disorder, which involves (a) assaying the expression of a polypeptide by measuring binding of an antibody composition of the present invention in cells or body fluid of an individual; (b) comparing the level of gene expression with a standard gene expression level, whereby an increase or decrease in the assayed polypeptide gene expression level compared to the standard expression level is indicative of a disorder.

The ability of a molecule to bind to activated integrin receptor can be determined, for example, by the ability of the putative ligand to bind to activated integrin receptor immunoadhesin coated on an assay plate. Specificity of binding can be determined by comparing binding to non-activated integrin receptor.

In one embodiment, antibody binding to activated integrin receptor can be assayed by either immobilizing the ligand or the receptor. For example, the assay can include immobilizing activated integrin receptor fused to a His tag onto Ni-activated NTA resin beads. Antibody can be added in an appropriate buffer and the beads incubated for a period of time at a given temperature. After washes to remove unbound material, the bound protein can be released with, for example, SDS, buffers with a high pH, and the like and analyzed.

Moreover, polypeptides or antibody compositions of the present invention can be used to treat disease. For example, patients can be administered a polypeptide or antibody compositions of the present invention in an effort to replace absent or decreased levels of the polypeptide (e.g., insulin), to supplement absent or decreased levels of a different polypeptide (e.g., hemoglobin S for hemoglobin B), to inhibit the activity of a polypeptide (e.g., an oncogene), to activate the activity of a polypeptide (e.g., by binding to a receptor), to reduce the activity of a membrane bound receptor by competing with it for free ligand (e.g., soluble TNF receptors used in reducing inflammation), or to bring about a desired response (e.g., blood vessel growth).

Similarly, antibody compositions of the present invention can also be used to treat disease. For example, administration of an antibody directed to a polypeptide of the present invention can bind and reduce overproduction of the polypeptide. Similarly, administration of an antibody can activate the polypeptide, such as by binding to a polypeptide bound to a membrane receptor.

Diagnostic Uses

Human antibodies and antibody compositions of the invention for use in diagnostic methods to identify metastatic tumor cells, e.g., malignant melanoma, are preferably produced using the methods described above. The methods result in virtually unlimited numbers of antibodies and antibody compositions of the invention of any epitope binding specificity and very high binding affinity to any desired antigen. In general, the higher the binding affinity of an antibody for its target, the more stringent wash conditions can be performed in an immunoassay to remove nonspecifically bound material without removing target antigen. Accordingly, antibodies and antibody compositions of the invention used in the above assays usually have binding affinities of at least 10⁸, 10⁹, 10¹⁰, 10¹¹ or 10¹² M⁻¹. Further, it is desirable that antibodies used as diagnostic reagents have a sufficient on-rate to reach equilibrium under standard conditions in at least 12 hours, preferably at least five hours and more preferably at least one hour.

Antibodies and antibody compositions of the invention used in the claimed methods preferably have a high immunoreactivity, that is, percentages of antibodies molecules that are correctly folded so that they can specifically bind their target antigen. Such can be achieved by expression of sequences encoding the antibodies in E. coli as described above. Such expression usually results in immunoreactivity of at least 80%, 90%, 95% or 99%.

Some methods of the invention employ polyclonal preparations of antibodies and antibody compositions of the invention as diagnostic reagents, and other methods employ monoclonal isolates. The use of polyclonal mixtures has a number of advantages with respect to compositions made of one monoclonal antibody. By binding to multiple sites on a target, polyclonal antibodies or other polypeptides can generate a stronger signal (for diagnostics) than a monoclonal that binds to a single site. Further, a polygonal preparation can bind to numerous variants of a prototypical target sequence (e.g., allelic variants, species variants, strain variants, drug-induced escape variants) whereas a monoclonal antibody can bind only to the prototypical sequence or a narrower range of variants thereto. However, monoclonal antibodies are advantageous for detecting a single antigen in the presence or potential presence of closely related antigens.

In methods employing polyclonal human antibodies prepared in accordance with the methods described above, the preparation typically contains an assortment of antibodies with different epitope specificities to the intended target antigen. In some methods employing monoclonal antibodies, it is desirable to have two antibodies of different epitope binding specificities. A difference in epitope binding specificities can be determined by a competition assay.

Although human antibodies can be used as diagnostic reagents for any kind of sample, they are most useful as diagnostic reagents for human samples. Samples can be obtained from any tissue or body fluid of a patient. Preferred sources of samples include, whole blood, plasma, semen, saliva, tears, urine, fecal material, sweat, buccal, skin and hair. Samples can also be obtained from biopsies of internal organs or from cancers. Samples can be obtained from clinical patients for diagnosis or research or can be obtained from undiseased individuals, as controls or for basic research.

The methods can be used for detecting any type of target antigen. Exemplary target antigens including bacterial, fungal and viral pathogens that cause human disease, such as. HIV, hepatitis (A, B, & C), influenza, herpes, Giardia, malaria, Leishmania, Staphylococcus aureus, Pseudomonas aeruginosa. Other target antigens are human proteins whose expression levels or compositions have been correlated with human disease or other phenotype. Examples of such antigens include adhesion proteins, hormones, growth factors, cellular receptors, autoantigens, autoantibodies, and amyloid deposits. Other targets of interest include tumor cell antigens, such as carcinoembryonic antigen. Other antigens of interest are class I and class II MHC antigens.

Human antibodies can be used to detect a given target in a variety of standard assay formats. Such formats include immunoprecipitation, Western blotting, ELISA, radioimmunoassay, and immunometric assays. See Harlow & Lane, supra; U.S. Pat. Nos. 3,791,932; 3,839,153; 3,850,752; 3,879,262; 4,034,074, 3,791,932; 3,817,837; 3,839,153; 3,850,752; 3,850,578; 3,853,987; 3,867,517; 3,879,262; 3,901,654; 3,935,074; 3,984,533; 3,996,345; 4,034,074; and 4,098,876, each incorporated herein by reference in their entirety and for all purposes.

Immunometric or sandwich assays are a preferred format. See U.S. Pat. Nos. 4,376,110, 4,486,530, 5,914,241, and 5,965,375, each incorporated herein by reference in their entirety and for all purposes. Such assays use one antibody or population of antibodies immobilized to a solid phase, and another antibody or population of antibodies in solution. Typically, the solution antibody or population of antibodies is labeled. If an antibody population is used, the population typically contains antibodies binding to different epitope specificities within the target antigen. Accordingly, the same population can be used for both solid phase and solution antibody. If monoclonal antibodies are used, first and second monoclonal antibodies having different binding specificities are used for the solid and solution phase. Solid phase and solution antibodies can be contacted with target antigen in either order or simultaneously. If the solid phase antibody is contacted first, the assay is referred to as being a forward assay. Conversely, if the solution antibody is contacted first, the assay is referred to as being a reverse assay. If target is contacted with both antibodies simultaneously, the assay is referred to as a simultaneous assay. After contacting the target with antibody, a sample is incubated for a period that usually varies from about 10 min to about 24 hr and is usually about 1 hr. A wash step is then performed to remove components of the sample not specifically bound to the antibody being used as a diagnostic reagent. When solid phase and solution antibodies are bound in separate steps, a wash can be performed after either or both binding steps. After washing, binding is quantified, typically by detecting label linked to the solid phase through binding of labeled solution antibody. Usually for a given pair of antibodies or populations of antibodies and given reaction conditions, a calibration curve is prepared from samples containing known concentrations of target antigen. Concentrations of antigen in samples being tested are then read by interpolation from the calibration curve. Analyte can be measured either from the amount of labeled solution antibody bound at equilibrium or by kinetic measurements of bound labeled solution antibody at a series of time points before equilibrium is reached. The slope of such a curve is a measure of the concentration of target in a sample

Suitable supports for use in the above methods include, for example, nitrocellulose membranes, nylon membranes, and derivatized nylon membranes, and also particles, such as agarose, a dextran-based gel, dipsticks, particulates, microspheres, magnetic particles, test tubes, microtiter wells, SEPHADEX™. (Amersham Pharmacia Biotech, Piscataway N.J., and the like. Immobilization can be by absorption or by covalent attachment. Optionally, antibodies can be joined to a linker molecule, such as biotin for attachment to a surface bound linker, such as avidin.

Labels

The particular label or detectable group used in the assay is not a critical aspect of the invention, so long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., Dynabeads™), fluorescent dyes (e.g., fluorescein isothiocyanate, Texas red, rhodamine, and the like), radiolabels (e.g., ³H, ¹⁴C, ³⁵S, ¹²⁵I, ¹²¹I, ¹¹²In, ⁹⁹mTc), other imaging agents such as microbubbles (for ultrasound imaging), ¹⁸F, ¹¹C, ¹⁵O, (for Positron emission tomography), ⁹⁹mTC, ¹¹¹In (for Single photon emission tomography), enzymes (e.g., horse radish peroxidase, alkaline phosphatase and others commonly used in an ELISA), and calorimetric labels such as colloidal gold or colored glass or plastic (e.g., polystyrene, polypropylene, latex, and the like) beads. Patents that described the use of such labels include U.S. Pat. Nos. 3,817,837; 3,850,752; 3,939,350; 3,996,345; 4,277,437; 4,275,149; and 4,366,241, each incorporated herein by reference in their entirety and for all purposes. See also Handbook of Fluorescent Probes and Research Chemicals (6^{th} Ed., Molecular Probes, Inc., Eugene Oreg.).

The label can be coupled directly or indirectly to the desired component of the assay according to methods well known in the art. As indicated above, a wide variety of labels can be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to an anti-ligand (e.g., streptavidin) molecule which is either inherently detectable or covalently bound to a signal system, such as a detectable enzyme, a fluorescent compound, or a chemiluminescent compound. A number of ligands and anti-ligands can be used. Where a ligand has a natural anti-ligand, for example, biotin, thyroxine, and cortisol, it can be used in conjunction with the labeled, naturally occurring anti-ligands. Alternatively, any haptenic or antigenic compound can be used in combination with an antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidoreductases, particularly peroxidases. Fluorescent compounds include fluorescein and its derivatives, rhodamine and its derivatives, dansyl, umbelliferone, and the like Chemiluminescent compounds include luciferin, and 2,3-dihydrophthalazinediones, e.g., luminol. For a review of various labeling or signal producing systems which can be used, see, U.S. Pat. No. 4,391,904, incorporated herein by reference in its entirety and for all purposes.

Means of detecting labels are well known to those of skill in the art. Thus, for example, where the label is a radioactive label, means for detection include a scintillation counter or photographic film as in autoradiography. Where the label is a fluorescent label, it can be detected by exciting the fluorochrome with the appropriate wavelength of light and detecting the resulting fluorescence. The fluorescence can be detected visually, by means of photographic film, by the use of electronic detectors such as charge coupled devices (CCDs) or photomultipliers and the like. Similarly, enzymatic labels can be detected by providing the appropriate substrates for the enzyme and detecting the resulting reaction product. Finally simple calorimetric labels can be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

Frequently, the activated integrin receptor or α₃β₁ integrin receptor proteins and antibodies to activated integrin receptor will be labeled by joining, either covalently or non-covalently, a substance which provides for a detectable signal.

Treatment Regimes

The invention provides pharmaceutical compositions comprising one or a combination of antibodies, e.g., antibodies to activated integrin receptor (monoclonal, polyclonal or single chain Fv; intact or binding fragments thereof) or antibody cytotoxin conjugates formulated together with a pharmaceutically acceptable carrier. Some compositions include a combination of multiple (e.g., two or more) monoclonal antibodies or antigen-binding portions thereof of the invention. In some compositions, each of the antibodies or antigen-binding portions thereof of the composition is a monoclonal antibody or a human sequence antibody that binds to a distinct, pre-selected epitope of an antigen.

In prophylactic applications, pharmaceutical compositions or medicaments of antibody cytotoxin conjugates are administered to a patient susceptible to, or otherwise at risk of a disease or condition (i.e., an immune disease) in an amount sufficient to eliminate or reduce the risk, lessen the severity, or delay the outset of the disease, including biochemical, histologic and/or behavioral symptoms of the disease, its complications and intermediate pathological phenotypes presenting during development of the disease. In therapeutic applications, compositions or medicants are administered to a patient suspected of, or already suffering from such a disease in an amount sufficient to cure, or at least partially arrest, the symptoms of the disease (biochemical, histologic and/or behavioral), including its complications and intermediate pathological phenotypes in development of the disease. An amount adequate to accomplish therapeutic or prophylactic treatment is defined as a therapeutically- or prophylactically-effective dose. In both prophylactic and therapeutic regimes, agents are usually administered in several dosages until a sufficient immune response has been achieved. Typically, the immune response is monitored and repeated dosages are given if the immune response starts to wane.

Antibody compositions that specifically binds to an activated integrin receptor on a metastatic tumor cell, antibody cytotoxin conjugates, ligand mimetics, derivatives and analogs thereof, useful in the present compositions and methods can be administered to a human patient per se, in the form of a stereoisomer, prodrug, pharmaceutically acceptable salt, hydrate, solvate, acid salt hydrate, N-oxide or isomorphic crystalline form thereof, or in the form of a pharmaceutical composition where the compound is mixed with suitable carriers or excipient(s) in a therapeutically effective amount, for example, cancer or metastatic cancer.

Pharmaceutically acceptable carriers are determined in part by the particular composition being administered, as well as by the particular method used to administer the composition. Accordingly, there is a wide variety of suitable formulations of pharmaceutical compositions for administering the antibody compositions (see, e.g., Remington's Pharmaceutical Sciences, Mack Publishing Co., Easton, Pa. 18^{th} ed., 1990, incorporated herein by reference). The pharmaceutical compositions generally comprise a differentially expressed protein, agonist or antagonist in a form suitable for administration to a patient. The pharmaceutical compositions are generally formulated as sterile, substantially isotonic and in full compliance with all Good Manufacturing Practice (GMP) regulations of the U.S. Food and Drug Administration.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

For example, “pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g., sodium and potassium, magnesium, calcium, and aluminum. Suitable organic salts include those formed with organic bases such as the amine bases, e.g., ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C₁₋₆ alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention can be present in more than one stereoisomeric form, and the naming of such compounds is intended to include all single stereoisomers and all mixtures (whether racemic or otherwise) of such stereoisomers.

A “therapeutically effective amount” means the amount that, when administered to a subject for treating a disease, is sufficient to effect treatment for that disease.

Except when noted, the terms “subject”, “patient” or “mammal” are used interchangeably and refer to mammals such as human patients and non-human primates, as well as experimental animals such as rabbits, rats, and mice, and other animals. Animals include all vertebrates, e.g., mammals and non-mammals, such as sheep, dogs, cows, chickens, amphibians, and reptiles. Accordingly, the term “subject” or “patient” as used herein means any mammalian patient or subject to which the compositions of the invention can be administered. In some embodiments of the present invention, the patient will be suffering from a condition that causes lowered resistance to disease, e.g., HIV. In an exemplary embodiment of the present invention, to identify subject patients for treatment with a pharmaceutical composition comprising one or more antibody-cytotoxin conjugate molecule according to the methods of the invention, accepted screening methods are employed to determine the status of an existing disease or condition in a subject or risk factors associated with a targeted or suspected disease or condition. These screening methods include, for example, screening examinations to determine whether a subject is suffering from a neoplastic disease. These and other routine methods allow the clinician to select subjects in need of therapy.

“Concomitant administration” of a known cancer therapeutic drug with a pharmaceutical composition of the present invention means administration of the antibody-cytotoxin conjugate molecule composition at such time that both the known drug and the composition of the present invention will have a therapeutic effect. Such concomitant administration can involve concurrent (i.e., at the same time), prior, or subsequent administration of the antimicrobial drug with respect to the administration of a compound of the present invention. A person of ordinary skill in the art, would have no difficulty determining the appropriate timing, sequence and dosages of administration for particular drugs and compositions of the present invention.

Effective Dosages

Effective doses of the antibody compositions of the present invention, e.g., antibodies to activated integrin receptor or antibody cytotoxin conjugates, for the treatment of immune-related conditions and diseases, e.g., metastatic cancer, described herein vary depending upon many different factors, including means of administration, target site, physiological state of the patient, whether the patient is human or an animal, other medications administered, and whether treatment is prophylactic or therapeutic. Usually, the patient is a human but nonhuman mammals including transgenic mammals can also be treated. Treatment dosages need to be titrated to optimize safety and efficacy.

For administration with an antibody, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months. In some methods, two or more monoclonal antibodies with different binding specificities are administered simultaneously, in which case the dosage of each antibody administered falls within the ranges indicated. Antibody is usually administered on multiple occasions. Intervals between single dosages can be weekly, monthly or yearly. Intervals can also be irregular as indicated by measuring blood levels of antibody in the patient. In some methods, dosage is adjusted to achieve a plasma antibody concentration of 1-1000 μg/ml and in some methods 25-300 μg/ml. Alternatively, antibody can be administered as a sustained release formulation, in which case less frequent administration is required. Dosage and frequency vary depending on the half-life of the antibody in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

Doses for nucleic acids encoding immunogens range from about 10 ng to 1 g, 100 ng to 100 mg, 1 μg to 10 mg, or 30-300 μg DNA per patient. Doses for infectious viral vectors vary from 10-100, or more, virions per dose.

Routes of Administration

Antibody compositions for inducing an immune response, e.g., antibodies to activated integrin receptor or antibody cytotoxin conjugates, for the treatment of immune-related conditions and diseases, e.g., metastatic cancer, can be administered by parenteral, topical, intravenous, oral, subcutaneous, intraarterial, intracranial, intraperitoneal, intranasal or intramuscular means for prophylactic as inhalants for antibody preparations targeting brain lesions, and/or therapeutic treatment. The most typical route of administration of an immunogenic agent is subcutaneous although other routes can be equally effective. The next most common route is intramuscular injection. This type of injection is most typically performed in the arm or leg muscles. In some methods, agents are injected directly into a particular tissue where deposits have accumulated, for example intracranial injection. Intramuscular injection on intravenous infusion are preferred for administration of antibody. In some methods, particular therapeutic antibodies are injected directly into the cranium. In some methods, antibodies are administered as a sustained release composition or device, such as a Medipad™ device.

Agents of the invention can optionally be administered in combination with other agents that are at least partly effective in treating various diseases including various immune-related diseases. In the case of tumor metastasis to the brain, agents of the invention can also be administered in conjunction with other agents that increase passage of the agents of the invention across the blood-brain barrier (BBB).

Formulation

Antibody compositions for inducing an immune response, e.g., antibodies to activated integrin receptor or antibody cytotoxin conjugates, for the treatment of immune-related conditions and diseases, e.g., metastatic cancer, are often administered as pharmaceutical compositions comprising an active therapeutic agent, i.e., and a variety of other pharmaceutically acceptable components. See Remington's Pharmaceutical Science, 1990 supra. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation can also include other carriers, adjuvants, or nontoxic, nontherapeutic, nonimmunogenic stabilizers and the like.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

For parenteral administration, compositions of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions. Antibodies can be administered in the form of a depot injection or implant preparation which can be formulated in such a manner as to permit a sustained release of the active ingredient. An exemplary composition comprises monoclonal antibody at 5 mg/mL, formulated in aqueous buffer consisting of 50 mM L-histidine, 150 mM NaCl, adjusted to pH 6.0 with HCl.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. These compositions take the form of solutions, suspensions, tablets, pills, capsules, sustained release formulations or powders and contain 10%-95% of active ingredient, preferably 25%-70%.

Topical application can result in transdermal or intradermal delivery. Topical administration can be facilitated by co-administration of the agent with cholera toxin or detoxified derivatives or subunits thereof or other similar bacterial toxins. Glenn et al., Nature 391: 851, 1998. Co-administration can be achieved by using the components as a mixture or as linked molecules obtained by chemical crosslinking or expression as a fusion protein.

Alternatively, transdermal delivery can be achieved using a skin patch or using transferosomes. Paul et al., Eur. J. Immunol. 25: 3521-24, 1995; Cevc et al., Biochem. Biophys. Acta 1368: 201-15, 1998.

The pharmaceutical compositions are generally formulated as sterile, substantially isotonic and in full compliance with all Good Manufacturing Practice (GMP) regulations of the U.S. Food and Drug Administration.

Toxicity

Preferably, a therapeutically effective dose of the antibody compositions or antibody cytotoxin conjugates described herein will provide therapeutic benefit without causing substantial toxicity.

Toxicity of the proteins described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch. 1).

Kits

Also within the scope of the invention are kits comprising the compositions (e.g., antibody cytotoxin conjugates, monoclonal antibodies, human sequence antibodies, human antibodies, multispecific and bispecific molecules) of the invention and instructions for use. The kit can further contain a least one additional reagent, or one or more additional human antibodies of the invention (e.g., a human antibody having a complementary activity which binds to an epitope in the antigen distinct from the first human antibody). Kits typically include a label indicating the intended use of the contents of the kit. The term label includes any writing, or recorded material supplied on or with the kit, or which otherwise accompanies the kit.

### EXEMPLARY EMBODIMENTS

The experiments described herein may also be found in Lilo et al. Chemistry & Biology 11:897, 2004, the contents of which are hereby incorporated by reference.

### Example 1

Expression and purification of Pan10. E. coli B834(DE3) (Novagen, Madison, Wis.) was selected as the expression host for transformation with plasmids pETflag-Pan10. Mao et al., Proc Natl Acad Sci USA, 96: 6953-6958, 1999. E. coli B834(DE3)/pETflag-Pan10 were grown in SB medium (30% peptone, 20% yeast extract, 10% MOPS) supplemented with 100 μM carbenicillin (RPI Corp., Mount Prospect, Ill.) at 37° C. to mid-log phase (OD₆₀₀ 0.65). Protein expression was induced by addition of 0.5 mM IPTG (RPI Corp.). The cultures were incubated for an additional 1 h at 37° C. and for 15 h at 26° C. A 4 L culture of IPTG-induced E. coli B834/pETflag-Pan10 was harvested by centrifugation. The resultant cell pellet was lysed using BugBuster Protein Extraction Reagent (Novagen) according to the vendor's instructions, while the supernatant was concentrated to ˜200 mL (EasyLoad, Masterflex from Millipore, Bedford, Mass.). Upon filtration through a 0.20M filter (Nalgene, Rochester, N.Y.), the cell-free lysate (˜100 mL) or the concentrated supernatant was loaded at a flow rate of 1 mL/min onto an Anti-Flag M2 affinity column (1.7×5 cm from Sigma, St. Louis, Mo.) previously equilibrated with PBS (phosphate-buffered saline). After washing with 100 mL of PBS, the flag-tagged Pan10 was eluted from the column with ˜20 mL of glycine buffer (0.1 M glycine pH 2.5) at a flow rate of 3 mL/min. The eluate was neutralized with ˜1 mL 1 M Tris Base. The level of purity was assessed by SDS-PAGE (10% Bis-Tris from Bio-Rad, Hercules, Calif.). A 4 L culture of IPTG-induced E. coli B834(DE3)/pETflag-Pan10 usually afforded 3.5-5 mg of purified protein, 60% of which was derived from the cell pellet.

Cell lines. The human pancreatic adenocarcinoma cell line SW1990 (ATCC, Manassas, Va.) was grown in Leibovitz's L-15 medium supplemented with 10% FCS (fetal calf serum). The normal human dermal fibroblasts (HdFa) from adult skin (Cascade Biologics, Portland, Oreg.) were grown in Medium 106 supplemented with low serum growth supplement.

SW1990-binding assay by whole-cell ELISA. SW1990 cells were trypsinized and resuspended in PBS to a concentration of 10⁶ cells/mL. 150 μL aliquots were poured in the wells of a 96-well ELISA plate (tissue culture treated, flat bottom from Corning Incorporated, Canton, N.Y.) and incubated at 37° C. to complete evaporation (note that two rows of wells contained medium only). The plate was then washed four times with 0.025% Tween 20 (Sigma, St. Louis, Mo.) in PBS, blocked with 1% BSA (bovine serum albumin from Sigma) in PBS, washed once with deionized water and pat-dried. 100 mL aliquots of serially diluted Pan10 (0.1-0 mg/mL, free or conjugated) in 1% BSA/PBS were added to the plate. One cell-free row was incubated with Pan10 while the other lacked Pan10. The plate was then incubated for 1 h at 37° C. and subsequently washed ten times with distilled water. 30 μL aliquots of M2 anti-flag/HRP (1.1 μg/mL, Sigma) in 1% BSA/PBS were added to all the wells and the plate was incubated for 1 h at 37° C. Finally, following extensive washing with distilled water, the plate was developed in the presence of TMB and H₂O₂ (Pierce, Rockford, Ill.) and read at 450 nm with a Spectra Max 250 plate reader (Molecular Devices, Sunnyvale, Calif.).

Pan10 mutation. Mutants Pan10S73C and Pan10S131C were generated by site-directed mutagenesis on template pETflag-Pan10 using standard PCR techniques. The PCR conditions used to introduce the mutations are as follows: denaturation at 95° C. for 10 min; 30 cycles of amplification; extension 2 min, 72° C.; denaturation, 95° C., 30 sec; annealing 60° C., 1 min and polishing, 72° C., 7 min. In the second stage the two halves of the mutated genes were overlapped (temperature program: denaturation at 95° C. for 10 min; 20 cycles of amplification; extension 2 min, 72° C.; denaturation, 95° C., 30 sec; annealing 50° C., 1 min and polishing, 72° C., 7 min). Finally in the third stage the product of overlap PCR was amplified using the two end primers (temperature program: denaturation at 95° C. for 10 min; 30 cycles of amplification; extension 2 min, 72° C.; denaturation, 95° C., 30 sec; annealing 55° C., 1 min and polishing, 72° C., 7 min). The amplified products were purified with PCR purification kit (Qiagen), digested with SfiI (New England BioLabs, Beverly, Mass.), purified and ligated (T4 DNA ligase, New England BioLabs) to SfiI-digested and purified pETflag. The sequence the Pan10 mutants was confirmed by full-length DNA sequencing (The Protein and Nucleic Acids Core Facility at The Scripps Research Institute, La Jolla, Calif.) using the end-primers.

Pan10 thiolation. Pan10 (4 mg/mL) in 50 mM triethanolamine, 1 mM EDTA and 150 mM NaCl, (pH 8.7) was incubated in the presence of a 10-fold stoichiometric excess of Traut's reagent (Pierce) for 5 h at 4° C., under constant agitation. The resultant mixture was desalted using PD-10 columns (Pharmacia, Peapack, N.J.), eluted with 50 mM Hepes pH 8 and concentrated by centrifugal ultrafiltration (YM 10,000 filter, Millipore). The concentration of free thiol in the desalted scFv solution was determined by Ellman's assay.

Ellman's assay. A 75% methanol solution of ˜30 μM thiolated scFv or standard dithiotreitol (DTT, ICN, Costa Mesa, Calif.) and 600 mM 5,5′-dithio-bis-(2-nitrobenzoic acid) (Ellman's reagent, Sigma) was centrifuged at 13000 rpm for 5 min. The supernatant was transferred to a 96-well ELISA plate (Fisher, Ottawa, Ontario) and the Abs₄₁₂ was read in a Spectra Max 25 plate reader (Molecular Devices). The concentration of free thiols was extrapolated from a standard curve obtained by plotting known concentrations of DTT versus the corresponding Abs₄₁₂.

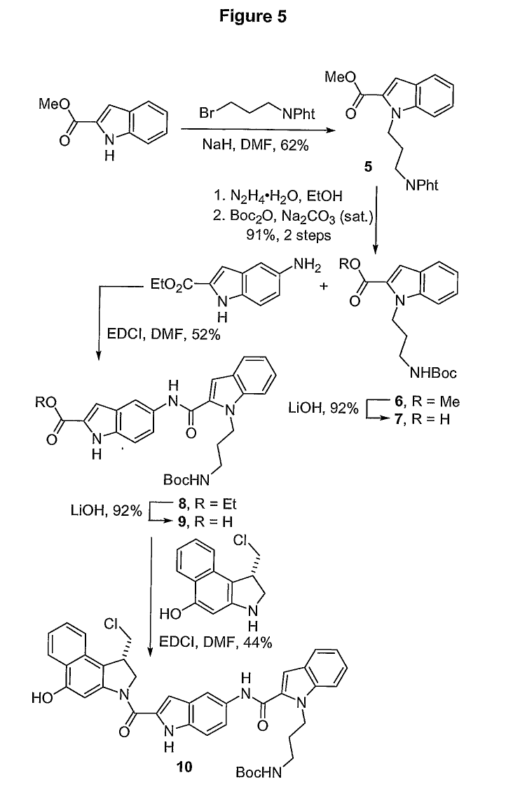

Synthesis of the analogs of duocarmycin SA (see FIG. 5 (all numbers appearing below in parentheses refer to FIG. 5)). All the chemicals utilized were purchased from Aldrich (St. Louis, Mo.). The synthesis of 3-(5-acetylindole-2-carbonyl)-1-(S)-(chloromethyl)-5-hydroxy-1,2-dihydro-3H-benz[e]indole (1) has been previously reported. Parrish et al., Bioorg Med Chem, 11: 3815-3838, 2003. The synthesis of the Boc-protected 3-[5-(1-(3-aminopropyl)indole-2-carbonyl)aminoindole-2-carbonyl]-1-(chloromethyl)-5-hydroxy-1,2-dihydro-3H-benz[e]indole (10) is as follows:

Methyl 1-(3-Phthalimidopropyl)indole-2-carboxylate (5). A solution of methyl indole-2-carboxylate (550 mg, 3.14 mmol) in dimethylformamide (31 mL) at 0° C. was treated with sodium hydride (60% suspension in mineral oil, 167 mg, 4.18 mmol) and allowed to warm at 25° C. over 30 min. The reaction mixture was cooled to 0° C. and treated with N-(3-bromopropyl)phthalimide (1.26 g, 4.71 mmol). The mixture was allowed to warm at 25° C. over 30 min and warmed at 55° C. for 30 min before being cooled and quenched with the addition of H₂O (30 mL). The reaction mixture was extracted with ethyl acetate (2×40 mL), and the combined organic layers were washed with water (40 mL), dried (Na₂SO₄), and concentrated in vacuo. Flash chromatography (silica gel, 0-50% ethyl acetate/hexane) afforded 5 in 62% yield.

Methyl 1-[3-(t-Butyloxycarbonyl)aminopropyl]indole-2-carboxylate (6). A suspension of 5 (500 mg, 1.39 mmol) in ethanol (14 mL) at 0° C. was treated with hydrazine (200 μL, 4.14 mmol). The reaction mixture was stirred at 0° C. for 1 h and then allowed to warm to 25° C. over 3 h before being concentrated in vacuo. The residue dissolved in chloroform (10 mL) was treated with t-butoxycarbonyl anhydride (602 mg, 2.76 mmol) and saturated aqueous sodium carbonate (10 mL). The reaction mixture was stirred at 25° C. for 12 h before being extracted with chloroform (3×100 mL). The combined organic layers were dried (Na₂SO₄), and concentrated in vacuo. Flash chromatography (silica gel, 10-30% ethyl acetate/hexane) afforded 6 in 91% yield.

Ethyl 5-(1-{3-[N-(t-Butyloxycarbonyl)amino]propyl}indole-2-carbonyl)-aminoindole-2-carboxylate (8). A solution of 6 (332 mg, 1.0 mmol) in 10 mL dioxane/H₂O (4:1) was treated with 4 N LiOH (1 mL) and the mixture was stirred at 25° C. for 15 h. 1 N Aqueous HCl (10 mL) was added, and the mixture was extracted with ethyl acetate (3×50 mL). The combined organic layers were dried (Na₂SO₄), and concentrated in vacuo to give 7 in 92% yield.

A solution of 7 (63.6 mg, 0.2 mmol) and ethyl 5-aminoindole-2-carboxylate (61.3 mg, 0.3 mmol) in dimethylformamide (4 mL) was treated with 1-(3-dimethylaminopropyl)-3-ethylcarbodiimide hydrochloride (115 mg, 0.6 mmol). The reaction mixture was stirred at 25° C. for 18 h and quenched with the addition of 15% aqueous citric acid (10 mL). The reaction mixture was extracted with ethyl acetate (75 mL and 2×25 mL), the combined organic layers were washed with saturated aqueous NaCl (3×10 mL), dried (Na₂SO₄), and concentrated in vacuo. Flash chromatography (silica gel, 33% ethyl acetate/hexane) afforded 8 in 52% yield.

5-(1-{3-[N-(t-Butyloxycarbonyl)amino]propyl}indole-2-carbonyl)indole-2-carboxylic Acid (9). A solution of 8 (50.5 mg, 0.1 mmol) in 2 mL dioxane/H₂O (4:1) was treated with 4 N LiOH (200 μL) and the mixture was stirred at 25° C. for 18 h. 0.5 N Aqueous HCl (5 mL) was added, and the mixture was extracted with ethyl acetate (2×30 mL). The combined organic layers were dried (Na₂SO₄) and concentrated in vacuo. Crystallization from tetrahydrofuran/hexane afforded 9 in 92% yield.

3-[5-(1-{3-[N-(t-Butyloxycarbonyl)amino]propyl}indole-2-carbonyl)aminoindole-2-carbonyl]-1-(chloromethyl)-5-hydroxy-1,2-dihydro-3H-benz[e]indole (10). A solution of (−)-seco-N-Boc-CBI (25 mg, 75 μmol, natural enantiomer) in 10 mL 4 N HCl (ethyl acetate) was stirred for 1 h at 25° C. before the solvent was removed under a stream of N₂. Boger et al., J Org Chem, 55: 5823-5832, 1990. The residue was dried under high vacuum for 3 h and 9 (39.5 mg, 83 μmol) was added. A solution of 1-(3-dimethylaminopropyl)-3-ethylcarbodiimide hydrochloride (43 mg, 225 μmol) in dimethylformamide (2 mL) was added and the reaction mixture was stirred for 14 h at 25° C. before the reaction mixture was concentrated in vacuo. Flash chromatography (silica gel, 20% tetrahydrofuran/hexane) afforded 10 in 44% yield.

Synthesis of 3. A mixture of 1 (3.0 mg, 7.2 μmol), maleimidopropionic acid hydrazide tetrahydrofuran salt (6 mg, 20 μmol), tetrahydrofuran (1 μL) and crushed 3 Å molecular sieves in 0.2 mL dimethylformamide was stirred overnight. Upon solvent evaporation the residue was dissolved in dichloromethane and purified by silica gel thin layer chromatography. 3 was obtained in 47% yield.

Synthesis of 4. Compound 10 (5 mg, 7.2 μmol) was treated with 50% trifluoroacetic acid in dichloromethane for 30 min. Upon trifluoroacetic acid evaporation the crude free amine was dissolved in 0.1 mL dimethylformamide and added to a dimethylformamide solution containing maleimidopropionic acid (2.0 mg, 12 μmol), O-benzotriazol-1-yl-N,N,N′,N′-tetramethyluronium hexafluoro-phosphate (4.2 mg, 11 mmol) and N-methylmorpholine (3.2 μL, 29 μmol). The mixture was stirred for 2 h and the solvent was evaporated. The residue was purified by silica gel thin layer chromatography. 4 was obtained in 57% yield.

Conjugation of thiolated Pan10. 3×1 μL aliquots of 20 mM fluorescein maleimide (Molecular Probes, Eugene, Oreg.), 3 or 4 in DMSO were added to 50 μL of Pan10 (˜4 mg/mL in 50 mM Hepes) at intervals of 2 min. The resultant reaction mixture was incubated on a shaker for 10 h at 4° C. Free dye or free drug were separated from the mixture of conjugated Pan10 and free Pan10 by size exclusion chromatography (PD-10 column, Pharmacia). The percentage yield of Pan10 conjugation to fluorescein was calculated by fitting the AbS492 nm and AbS280 nm (Ultrospec 2000, Pharmacia) of the desalted mixture into equation 1:

(Abs₄₉₂/59880^{a}×100)/((Abs₂₈₀−(0.2×Abs₄₉₂)/1.35^{b})/scFv-FM MW) Equation 1

^{a}: ε₄₉₂ experimentally determined for fluorescein-maleimide

^{b}: ε₂₈₀ typically adopted for IgG

The ratio of 3 or 4 to scFv was indirectly determined by calculating the amount of residual free scFv after the drug conjugation step. The mixture of Pan10-drug conjugate and free Pan10 was reacted with fluorescein-maleimide and the amount of fluorescein-Pan10 (determined as described above) was assumed to correspond to the entire amount of Pan10 not bound to the drug. The percentage yield of Pan10 conjugation to 3 or 4 was calculated by fitting the Abs_{492 nm} and Abs₂₈₀ₙₘ of the desalted mixture obtained after the conjugation of scFv-drug+free scFv to the maleimide derivative of fluorescein into equation 2:

100−((Abs₄₉₂/59000^{a}×100)/((Abs₂₈₀−(0.2×Abs₄₉₂)/1.35^{b})/scFv-FM MW)) Equation 2

Mass spectrometry. Matrix-assisted laser desorption/ionization mass spectrometry (MALDI-MS) was performed on a Voyager DE Biospectrometry Workstation (PerSeptive Biosystems, Framingham, Mass.) in the linear mode using a nitrogen laser (337 nm) and sinapinic acid (Sigma) as matrix. Matrix solutions were prepared fresh daily as saturated solutions in a 1:1 mixture of acetonitrile and 0.1% aqueous trifluoroacetic acid. Samples were prepared for MALDI-MS analysis by diluting the desalted protein solution 1:10 with matrix and depositing 0.7 μL the resulting suspension directly onto a stainless steel MALDI target well. The obtained masses were calibrated using two-point external calibration with equine cytochrome C and rabbit muscle aldolase (Sigma). All spectra were collected in positive ion mode with 140-ns delayed extraction and summed over approximately 50 laser shots.

One-pot antibody conjugation. Pan10 (4 mg/mL) in 50 mM triethanolamine, 1 mM EDTA and 150 mM NaCl, (pH 8.7) was incubated 8 hours at 4° C., under constant agitation, in the presence of a 10-fold stoichiometric excess of Traut's reagent (Pierce) and an equal excess of either fluorescein maleimide (Molecular Probes), 3 or 4. The resultant mixture was desalted using PD-10 columns, eluted with PBS and concentrated by centrifugal ultrafiltration (YM 10,000 filter, Millipore) to ˜4 mg/mL.

Confocal microscopy. SW1990 or HdFa cells were trypsinized, resuspended in PBS and counted. 10⁴-10⁵ cells were seeded into the wells of a chamber slide (Nunc, Naperville, Ill.) and allowed to attach for 24 h at 37° C. Upon changing the medium (500 μL/well), 10 μL of ˜3 mg/mL concentrated Pan10-fluorescein or 92H2-fluorescein (negative control) was added and the cells were incubated for 30 min, 1, 2 or 3 h at 37° C. The cells were then washed 10 times with their respective medium and once with PBS, then they were fixed and permeabilized with 95% ethanol for 5 min, washed once with PBS, stained with propidium iodine (Sigma, 1:50 diluted in PBS) for 1 min, washed five times with PBS and sealed with a coverslip upon addition of antifade solution (Slow Fade, Molecular Probes). The slides were observed with a laser scanning confocal microscope (MRC1024, Bio-Rad).

FACS analysis. SW1990 or HdFa cells were trypsinized, washed in cold PBS and aliquoted (˜5×10⁵ cells/tube). The primary antibody (either W6/32, Novus Biologicals, Littleton, Colo.; P1B5, Chemicon, Temecula, Calif.; or P5D2, Chemicon) was then added (final concentration 10 μg/mL) and the incubation carried on for 45 min on ice. The cells were then washed with cold PBS and incubated in the presence of FITC-labeled goat anti-mouse Ab (Pierce, Rockford, Ill.) on ice for 45 min. A final wash with cold PBS was followed by PI counter stain and analysis (FACScan, Becton Dickinson, Franklin Lakes, N.J.).

Inverted microscopy. SW1990 cells were trypsinized, resuspended in PBS and counted. 10⁴-10⁵ cells in 500 μL of growth medium were seeded into the wells of a chamber slide (Nunc) and allowed to attach for 24 h at 37° C. The old medium was than replaced by medium containing 400 nM of either Pan10-3, Pan10-4, Pan10-FM or wt-Pan10. Cells were then observed with an inverted microscope (Zeiss 1 mm, Thornwood, N.Y.) every day for 7 days.

Cell proliferation assay. The cytotoxicity of scFv-drug or free drug was quantified by using the Vybrant MTT cell proliferation assay kit (Molecular Probes). Assays were performed using 48-well microtiter plates containing 2×10⁴ SW1990 (HdFa) cells/well in 300 μL of phenol-free growth medium. Cells were allowed to attach to the wells for 12 hours. For determination of IC₅₀, cells were incubated for 3 or 12 h at 37° C. with various concentrations of Pan10-drug conjugates, maleimide derivatives or free drugs. Then the incubation was continued in conjugate/drug-free medium and the MTT assay was performed at the end of the seventh day. Medium was replaced with 100 μL of fresh medium containing 1.2 mM MTT and the incubation continued for three more hours. The cells were then lysed by adding 100 μL of a 10 mM solution of HCL containing SDS (100 mg/mL). The cell lysis was allowed to proceed for a period of 8 hours at the end of which the plate was centrifuged at 3000 rpm for 3 min and the supernatant transferred in a 96-well plate and read at 570 nM. Each assay included a negative control of cells treated with free Pan10 and a positive control lacking cells. All assays were performed at least twice. A set of 8 data points was obtained with various concentration of cytotoxicity agent. In order to obtain the IC₅₀ values, data points from each set were fit to the sigmoidal dose-response curve defined by Equation 3 using Grafit5 (Leatherbarrow R. J. 2003. Grafit version 5.08, Erithieus Software Ltd, Staines, England).

y^{a}=min y+[(max y−min y)/(1+(IC₅₀/x^{b})^{slope})] (Equation 3)

^{a}y=% of live cells

^{b}x=concentration of drug (drug-scF)

Data points which were outliers (typically 1-2 per experiment) were discarded.

### Example 2

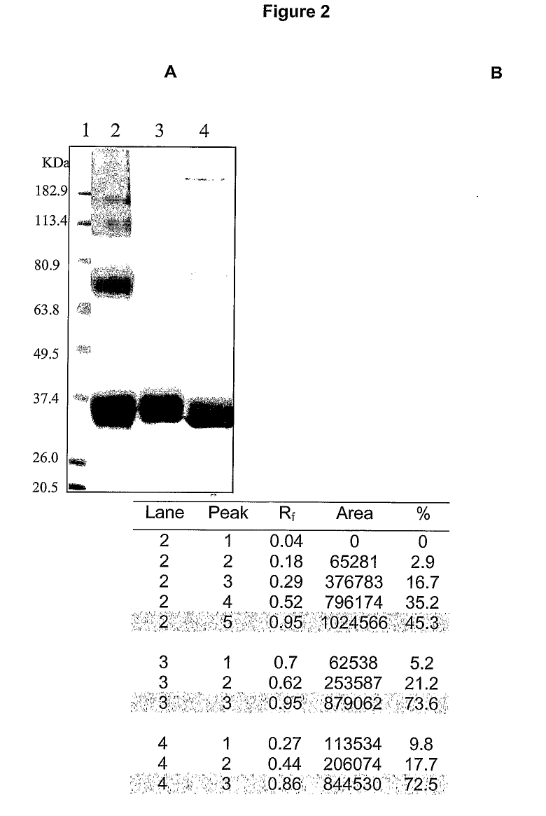

Pan10 expression, purification and site-directed mutagenesis. To use Pan10 as a tool for the delivery of duocarmycin analogs to malignant cancer cells, phage-free Pan10 was expressed as a scFv of 27,868 kDa (Table 1) and purified to homogeneity (FIG. 2, lane 4). Since typical V_{L} and V_{H} domains each possess a buried single disulfide linkage, but no free cysteines, we investigated several strategies intended to make available free thiol groups on the surface of Pan10 and to conjugate the modified scFv to maleimide-derivatized drugs. Padlan, In Molecular Biology Intelligence Unit: Antibody-Antigen complexes (Austin, Tex., Landes, R. G.), 17-30, 1994.

TABLE 1
Results of mass spectrometry analysis

| | | | Molar ratios^{a} |
|---|---|---|---|
| | Calculated | Experimental | (Pan10:FM/drug- |
| Analyte | MW (g/mol) | mass (m/z) | maleimide) |
| Pan10 | 27610 | 27868 | — |
| Pan10-FM | 28216 | 28388 | 0.997 |
| Pan10-3 | 28297 | 28634 | 1.003 |
| Pan10-4 | 28456 | 28545 | 0.994 |

^{a}Measured from MR = MW_{conjugate} − MW_{Pan10}/MW_{FM/drug-maleimide} using MALDI-measured MWs

An initial approach was aimed at single site-specific conjugation using a cysteine incorporated into the wild-type Pan10 sequence by site-directed mutagenesis. In an attempt to preserve the scFv binding affinity, the cysteine residue was first introduced into the linker region of Pan10. Additionally, a commercially available maleimide-derivatized fluorescein (FM) was used as a sensitive reagent to optimize and quantify conjugation protocols. When any one of the linker residues S131, G130, G128 or G127 was mutated to cysteine, the efficiency of conjugation of the mutants with FM was only similar to the wild-type Pan10, which was likely due to the linker region and cysteine residue being sequestered within the Pan10 structure. Several other residues were chosen that appeared surface exposed, according to a WAM (Web Antibody Modeling c/o University of Bath At Swindon, Oakfield Campus, Marlowe Avenue, Walcot Swindon Wilts, UK) theoretical structure of Pan10. To preserve the tumor cell binding and internalizing ability of Pan10, only framework residues were considered.

Improved FM conjugation was achieved when the more exposed residues S73 or S197 were mutated, the improvement likely due to the accessibility of the linker residues. The efficiency of conjugation achieved was at best 68%.

Chemical modification of scFv Pan10. Insertion of free cysteines by site-directed mutagenesis, presents several drawbacks: if the mutated residue is solvent accessible it will likely undergo oxidation or induce dimerization, requiring an additional reduction-purification step, the subsequent reactions of which must be carried out in an inert atmosphere. Yang et al., Protein Eng, 16, 761-770, 2003. The chemical addition of thiol groups onto Pan10 was instead investigated.

Initially, the thiolation and conjugation to maleimide-derivatized molecules were performed in two separate steps: the free thiol groups were introduced by reacting Pan10 with 2-iminothiolane (Traut's reagent), an amine scavenger that reacts with lysine residues. The presence of twelve lysines in the Pan10 sequence had the potential to lead to a massive and potentially harmful modification; however, because ten of the lysines were in the framework regions, therefore their modification would unlikely affect Pan10 binding to integrin α₃β₁. Whole-cell ELISA (enzyme-linked immunosorbent assay) revealed that binding of wild-type Pan10 and thiolated Pan10 were virtually indistinguishable. Non-reducing SDS-PAGE (sodium dodecyl sulphate polyacrylamide gel electrophoresis) analysis of the thiolated Pan10 (FIG. 2) revealed time-dependent formation of dimers and higher polymers (FIG. 2A, lane 2), which caused a progressive reduction of the number of free thiol groups available for drug conjugation. To avoid this problem, the subsequent drug conjugation step was performed immediately after thiolation, thereby improving the coupling efficiency and reducing dimerization. A further improvement was obtained with the use of a one-step procedure, where thiolation and conjugation occurred in one pot. SDS-PAGE analysis of Pan10 modified using this method revealed only a negligible formation of dimers (FIG. 2A, lane 3). Other scFvs may utilize this procedure.

Maleimide-derivatized drugs. The maleimide moiety was attached to duocarmycin SA analog 1 via an acid-labile hydrazone linkage to give the maleimide derivative 3 (FIG. 1). The hydrazone linkage method is widely utilized in antibody-drug conjugates as a way of controlled release of the cytotoxic drug upon internalization into lysosomes where the pH value is slightly lower (pH=5.0-5.5) then in the cytosol. Kaneko et al., Bioconjug Chem, 2: 133-141, 1991. This strategy has proven clinically effective in many instances such as in the development of BR96-DOX by Bristol-Myers Squibb and the design of Mylotarg by Wyeth. As a comparison, we derivatized duocarmycin analog 2 through a pH-insensitive amide bond linkage, producing the maleimide derivative 4 (FIG. 1).

Pan10 conjugation to maleimide-derivatized molecules. As noted above, thiolated Pan10 was initially conjugated to FM to give Pan10-FM in order to test and directly visualize internalization by pancreatic cancer cells. Subsequently, the thiolated Pan10 was conjugated to maleimide derivatives 3 and 4 obtaining conjugates Pan10-3 and Pan10-4, respectively. The ratio of either fluorescein (conjugation efficiency measured by UV/Vis spectrometry and matrix-assisted laser desorption/ionization mass spectrometry (MALDI-MS)), 3 or 4 (conjugation efficiency measured by MALDI-MS) to Pan10 was found to be approximately 1:1 using our two-step coupling procedure (Table 1). When conjugation and thiolation were performed in a single step, the ratio of fluorescein to Pan10 was 2:1 (conjugation efficiency measured by UV/Vis spectrometry only). The difference in conjugation efficiency is possibly due to the polymerization of the thiolated Pan10 in the absence of thiol-quenching small molecules. Indeed, several additional higher molecular weight species were detected by SDS-PAGE (FIG. 2) and size exclusion chromatography of Pan10-drug conjugates obtained in two separate steps.

The one-pot scFv conjugation method has been tested on other scFvs (αᵥβ₃ specific antibodies Bc-12 and Bc-15 [Felding-Habermann et al. Manuscript submitted for publication] and cocaine-specific antibody 92H2) affording a maximum ratio of fluorescein:protein of 3:1 without loss of antigen-binding activity (data not shown). Redwan et al., Biotechnol Bioeng, 82: 612-618, 2003. Such a conjugation method is applicable to a vast array of scFvs.

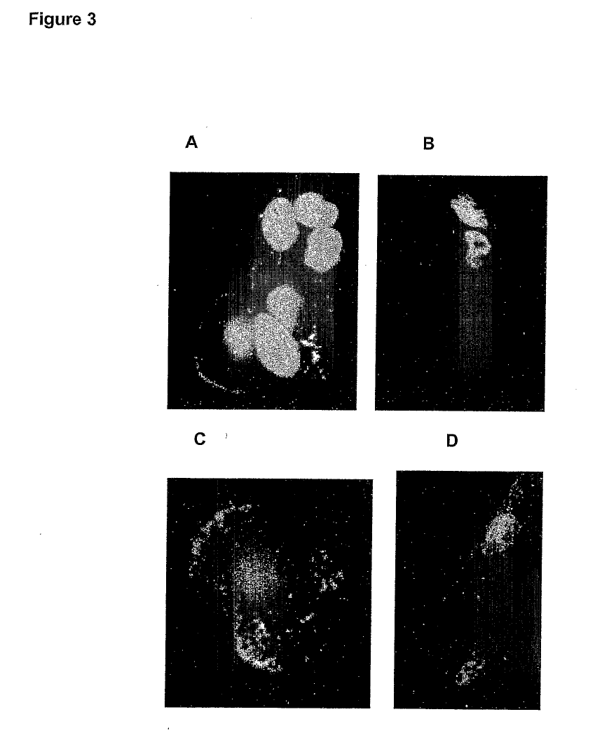

Biological activity of Pan10 conjugates. Several methods were employed to explore the biological activity of our Pan10 conjugates. Confocal microscopy analysis was used to investigate the specificity of the interaction of the Pan10-FM with SW1990 cells versus the normal human dermal fibroblast cell line (HdFa). Our results showed that Pan10-FM was internalized by SW1990 cells in a time-dependent fashion (FIG. 3). Moreover, after the second hour of incubation, internalization in these cancerous cells was much more pronounced than in non-cancerous HdFa. These findings confirm that the Pan10-FM conjugate retains the wild-type activity of Pan10 and provide evidence that in pancreatic cancer cells the overexpression of integrin α₃β₁ allows some selectivity versus HdFa used as a model for a non-cancer cell type.

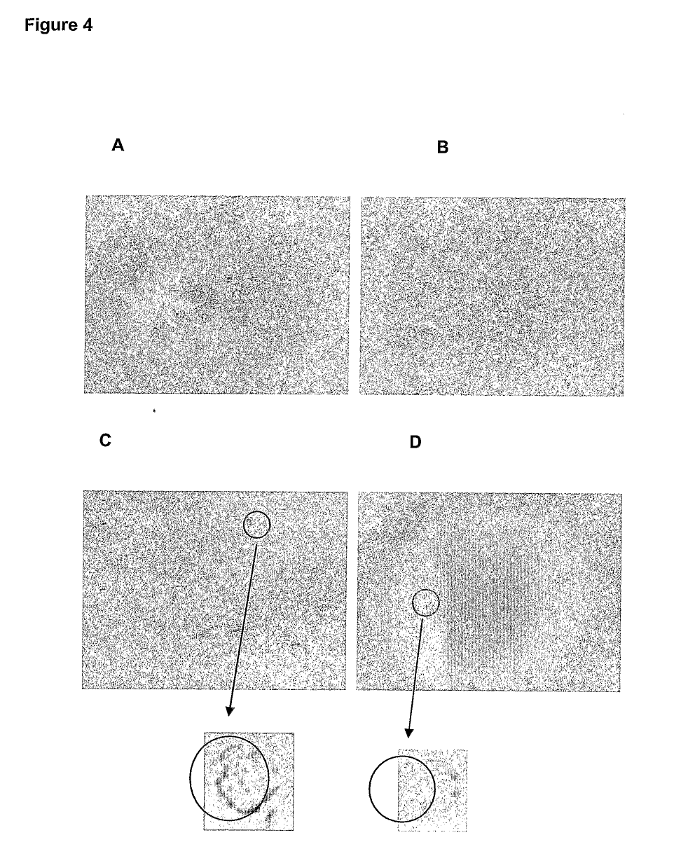

SW1990 cells treated with Pan10, Pan10-FM, Pan10-3 or Pan10-4 were examined by inverted microscopy for a qualitative determination of the effect of the drug conjugates on the cell viability. After seven days in culture, the cells treated with Pan10 or Pan10-FM had expanded into healthy colonies, whereas the cells treated with Pan10-drug conjugates had either died or showed excessive vacuolization, indicating advanced apoptosis (FIG. 4). The cytotoxic effect of the Pan10-drug conjugates in comparison with the toxicity of the free drugs was then quantified by the MTT (3-(4,5-dimethylthiazol-2-yl)-2,5,-diphenyltetrazolium bromide) cell-proliferation assay. Liu et al., J Neurochem, 69: 581-593, 1997; Berridge et al., Arch Biochem Biophys, 303: 474-482, 1993; Vistica et al., Cancer Res, 51: 2515-2520, 1991. SW1990 pancreatic carcinoma cells were seeded and allowed to attach in growth medium overnight. The cultures were then treated for either 3 or 12 h with increasing concentrations of free drugs or Pan10-drug conjugates.

After seven days, the number of viable cells indicated a clear cytostatic/cytotoxic effect of Pan10-drug conjugates, especially after the 12 h drug exposure time (Table 2). The IC₅₀ (inhibitory concentration 50%) values measured for the free drugs (Table 2) were two to three orders of magnitude higher than the previously obtained values (2=30 pM and 1=2 pM [obtained as for 2]). Parrish et al., Bioorg Med Chem, 11: 3815-3838, 2003. This inconsistency is probably due to a difference in the cell line used, duration of drug exposure, and cytotoxicity assay chosen. In the present study, the free drugs had a more potent cytotoxic effect than the corresponding Pan10-drug conjugates, especially after a short exposure time. This effect is likely due to a more immediate availability of the free drug in the nucleus, where DNA is the site of action, upon diffusion through the plasma and nuclear membranes. Further evidence of this came from the observation that the difference in efficacy between free drug and Pan10-drug conjugate was significantly reduced when the incubation time was extended. After a 12 h incubation period, the Pan10-4 conjugate was as effective as the free compound 2.

TABLE 2
Results of MTT assay on SW1990 cells

| Cytotoxic agent | IC₅₀ (nM) | IC₅₀ (nM)^{d} |
|---|---|---|
| 1 | 1.4 ± 0.2^{b} | 0.43 ± 0.17 |
| Pan10-3 (1:1)^{a} | 94.3 ± 3.6^{c} | 2.7 ± 0.3 |
| Pan10-3 (1:2)^{a} | 251.3 ± 75.8^{b} | 22.6 ± 3.8 |
| 2 | 32.1 ± 13.1^{b} | 4.3 ± 0.2 |
| Pan10-4 (1:1)^{a} | 97.9 ± 38.6^{c} | 4.4 ± 0.7 |
| Pan10-4 (1:2)^{a} | 1528 ± 369.4^{b} | 180.8 ± 30.6 |

^{a}ratio scFv to drug
^{b}average of four experiments. 3 h incubation with drug
^{c}average of two experiments. 3 h incubation with drug
^{d}average of two experiments. 12 h incubation with drug

Pan10-3 (scFv:drug=1:1) displayed a cytotoxicity similar to Pan10-4 (scFv:drug=1:1). This result, together with the lower cytotoxicity observed for conjugates carrying two drug molecules per molecule of scFv, suggests that there is no advantage in derivatizing the drug through the hydrazone linkage. The results also imply that the mechanism of endocytosis of the Pan 10 conjugates can not involve transfer into a low pH environment and that upon cell internalization the drug remains linked either to the intact Pan10 or to peptides derived from the intracellular proteolysis of Pan10. The residual activity of such hypothetical complexes is not surprising since the tether used between the scFv and the drug is probably long enough to allow for interaction with the DNA target and preservation of cytotoxicity. Indeed, a conjugate in which drug release from the scFv/scFv-derived peptides is not required for cytotoxic action could be advantageous, particularly within the context of a cell internalization mechanism. In this way, the scFv/peptide-drug compared to free drug might be trapped more effectively within the cell through reduced passive (diffusional) and active efflux processes. Overall, this mechanism would lead to the time-dependent accumulation of high intracellular concentrations of drug, affording the potential for efficient cancer cell killing, an excellent therapeutic index and a decreased likelihood of acquiring drug resistance.

Finally, in testing the Pan10-3 and the Pan10-4 conjugates on the normal HdFa cells, cytotoxicity was observed with an IC₅₀˜3 and 5-fold higher, respectively, than those against the SW1990 cancer cells, whereas the free 1 had roughly the same IC₅₀ values against both cells lines. There can be a correlation between the result and a measurement by FACS (fluorescence-activated cell sorting) that showed a 5-fold greater level of α₃ integrin expression on SW1990 cell compared to HdFa cells.

### Example 3

Chemically modified anti-integrin α₃β₁ scFv Pan10 containing free thiols can be conjugated to maleimide-derivatized analogs of the potent cytotoxic agent duocarmycin SA. Antibody Pan10 conjugates conserve the ability to penetrate cells expressing integrin α₃β₁. In particular Pan10-drug conjugates show excellent cytotoxic effects on pancreatic carcinoma cells in vitro. This first step is important considering the unique advantage of the scFv conjugates compared to the free drugs described herein, which are extremely potent but not clinically viable anticancer agents. The conjugates can deliver these drug molecules more specifically to the interior of cancer cells which overexpress integrin α₃β₁ and efficient delivery of antibody drug conjugates should allow for reduced therapeutic drug exposure and enhanced efficacy. Using such a strategy, experiments will further elaborate the potential for scFv-drug designs in cancer treatment.

All publications and patent applications cited in this specification are herein incorporated by reference in their entirety for all purposes as if each individual publication or patent application were specifically and individually indicated to be incorporated by reference for all purposes.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

We claim:

1. A method for treating a neoplastic disease in a mammal comprising:
providing an antibody-cytotoxin conjugate with an acid-stable covalent linkage between an antibody and a cytotoxin, and
administering said antibody-cytotoxin conjugate to the mammal so that said conjugate is internalized within a cell of the mammal to treat said neoplastic disease.

2. The method of claim 1 wherein said cytotoxin is an antitumor antibiotic, duocarmycin, duocarmycin A, duocarmycin SA, or an analog thereof.

3. The method of claim 1 wherein said acid-stable linkage is an amide linkage.

4. The method of claim 3 wherein said amide linkage is an N-substituted amide linkage.

5. The method of claim 1 wherein the antibody portion of said conjugate specifically binds to an activated integrin receptor.

6. The method of claim 5 wherein said activated integrin receptor is differentially produced on a cell in a metastatic state as compared to a similar, non-metastatic cell.

7. The method of claim 6 wherein said activated integrin receptor is an α₃β₁ integrin receptor or an αvβ3 integrin receptor.

8. The method of claim 1 wherein the antibody portion of said conjugate is a single chain Fv antibody.

9. The method of claim 1 wherein said neoplastic disease is selected from solid tumor, hematological malignancy, leukemia, colorectal cancer, benign or malignant breast cancer, uterine cancer, uterine leiomyomas, ovarian cancer, endometrial cancer, polycystic ovary syndrome, endometrial polyps, squamous cell carcinoma, squamous cell carcinoma of the head and neck, hepatocellular carcinoma, intrahepatic metastasis of hepatocellular carcinoma, prostate cancer, prostatic hypertrophy, pituitary cancer, adenomyosis, adenocarcinoma, pancreatic adenocarcinoma, meningioma, melanoma, bone cancer, multiple myeloma, CNS cancer, glioma, or astroblastoma.

10. A method for treating a neoplastic disease in a mammal comprising:
providing an antibody-cytotoxin conjugate with an acid-labile covalent linkage between an antibody and a cytotoxin, and
administering said antibody-cytotoxin conjugate to the mammal so that said conjugate is internalized within a cell of the mammal to treat said neoplastic disease.

11. The method of claim 10 wherein said cytotoxin is an antitumor antibiotic, duocarmycin, duocarmycin A, duocarmycin SA, or an analog thereof.

12. The method of claim 10 wherein said acid-labile covalent linkage is a hydrazone linkage.

13. The method of claim 12, further comprising administering said antibody-antitumor antibiotic conjugate so that said conjugate is internalized within a cell of the mammal by cleavage of an acid-labile hydrazone linkage.

14. The method of claim 10 wherein said antibody specifically binds to an activated integrin receptor.

15. The method of claim 14 wherein said activated integrin receptor is differentially produced on a cell in a metastatic state as compared to a similar, non-metastatic cell.

16. The method of claim 15 wherein said activated integrin receptor is an α₃β₁ integrin receptor or an αvβ3 integrin receptor.

17. The method of claim 10 wherein the antibody portion of said conjugate is a single chain Fv antibody.

18. The method of claim 10 wherein said neoplastic disease is selected from solid tumor, hematological malignancy, leukemia, colorectal cancer, benign or malignant breast cancer, uterine cancer, uterine leiomyomas, ovarian cancer, endometrial cancer, polycystic ovary syndrome, endometrial polyps, squamous cell carcinoma, squamous cell carcinoma of the head and neck, hepatocellular carcinoma, intrahepatic metastasis of hepatocellular carcinoma, prostate cancer, prostatic hypertrophy, pituitary cancer, adenomyosis, adenocarcinoma, pancreatic adenocarcinoma, meningioma, melanoma, bone cancer, multiple myeloma, CNS cancer, glioma, or astroblastoma.

19. A method of synthesizing an antibody-cytotoxin conjugate molecule comprising:
introducing into a single vessel an antibody, a thiolating reagent, and a maleimide-derivatized cytotoxin;
contacting said antibody with said thiolating reagent to form a thiolated antibody; and
contacting said thiolated antibody with said maleimide-derivatized cytotoxin to form an antibody-cytotoxin conjugate molecule.

20. The method of claim 19 wherein said maleimide-derivatized cytotoxin comprises an acid-labile hydrazone linkage between maleimide and the cytotoxin.

21. The method of claim 19 wherein said maleimide-derivatized cytotoxin comprises an amide bond linkage between maleimide and the cytotoxin.

22. The method of claim 19, wherein said cytotoxin is an antitumor antibiotic, duocarmycin, duocarmycin A, duocarmycin SA, or an analog thereof.

23. The method of claim 22 wherein said antitumor antibiotic is a carbonyl-substituted CBI-indole analog of duocarmycin SA.

24. The method of claim 22 wherein said antitumor antibiotic is an amide-substituted CBI-indole analog of duocarmycin SA.

25. The method of claim 23 wherein said maleimide-derivatized cytotoxin is 1-[3-(N′-{1-[2-(1-Chloromethyl-5-hydroxy-1,2-dihydro-3H-benzo[e]indole-3-carbonyl)-1H-indol-5-yl]-ethylidene}-hydrazino)-3-oxo-1-propyl]maleimide.

26. The method of claim 24 wherein said maleimide-derivatized cytotoxin is 3-[5-[1-{3-[3-(2,5-dioxo-2,5-dihydropyrrol-1-yl)propionylamino]propyl}indole-2-carbonyl]aminoindole-2-carbonyl]-(1-chloromethyl)-5-hydroxy-1,2-dihydro-3H-benzo[e]indole.

27. The method of claim 19, wherein said thiolating reagent is 2-iminothiolane.

28. The method of claim 19, wherein said antibody is a single chain Fv antibody.

29. A compound 3-[5-(1-(3-aminopropyl)indole-2-carbonyl)aminoindole-2-carbonyl]-1-(chloromethyl)-5-hydroxy-1,2-dihydro-3H-benz[e]indole.

30. A compound of Formula I: 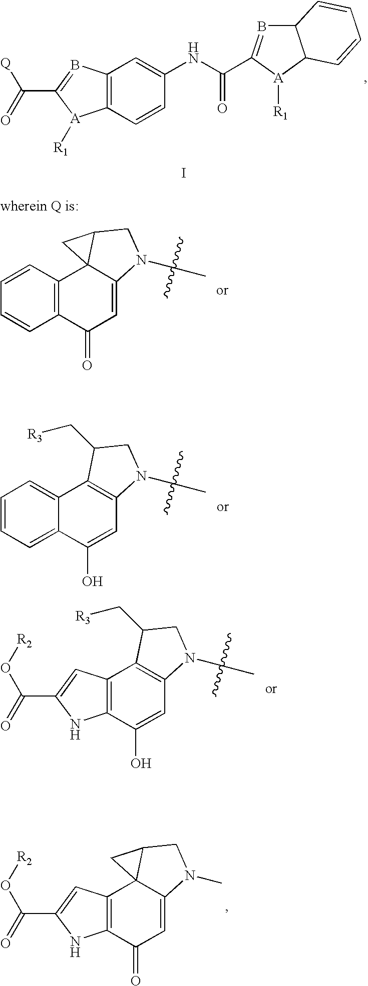
each A is independently NR₁, O or S, provided that at least one A is NR₁;
each B is independently C or N;
R₁ is independently H or —(CH₂)ₙ—N(H)R₄, provided that one R₁ is H and the other is —(CH₂)ₙ—N(H)R₅;
R₂ is alkyl;
R₃ is halogen;
R₄ is H or —C(═O)—(CH₂)ₘ—N-maleimide;
m is 2, 3, 4, 5 or 6; and
n is 2, 3, 4, 5 or 6;
or a stereoisomer, prodrug, pharmaceutically acceptable salt, hydrate, solvate, acid salt hydrate, N-oxide or isomorphic crystalline form thereof.

31. The compound of claim 30 wherein R₂ is C₁ to C₆ alkyl.

32. The compound of claim 30, wherein halogen is Cl, Br, or F.

33. The compound 3-[5-(1-(3-aminopropyl)indole-2-carbonyl)aminoindole-2-carbonyl]-1-(chloromethyl)-5-hydroxy-1,2-dihydro-3H-benz[e]indole.

34. The compound 3-[5-[1-{3-[3-(2,5-dioxo-2,5-dihydropyrrol-1-yl)propionylamino]propyl}indole-2-carbonyl]aminoindole-2-carbonyl]-(1-chloromethyl)-5-hydroxy-1,2-dihydro-3H-benzo[e]indole.

35. A pharmaceutical composition comprising at least one pharmaceutically acceptable carrier or excipient and an effective amount of the compound of claim 30, wherein the maleimide moiety is conjugated to a single chain Fv antibody.

36. The composition of claim 35 wherein said single chain Fv antibody is capable of binding to an integrin receptor.

37. The composition of claim 36 wherein said integrin receptor is an-α₃β₁ integrin receptor or an αvβ3 integrin receptor.

38. A method comprising administering to a mammal the composition of claim 36.

39. A method for alleviating a disease state in a mammal believed to be responsive to treatment with an antibody conjugated to a amide-substituted CBI-indole analog of duocarmycin SA comprising the step of administering to the mammal a therapeutic amount of the composition of claim 36.

40. The method of claim 36 wherein said compound is 3-[5-(1-(3-aminopropyl)indole-2-carbonyl)aminoindole-2-carbonyl]-1-(chloromethyl)-5-hydroxy-1,2-dihydro-3H-benz[e]indole.

41. The method of claim 39, wherein said disease state is neoplastic disease.

42. The method of claim 40 wherein the neoplastic disease is selected from solid tumor, hematological malignancy, leukemia, colorectal cancer, benign or malignant breast cancer, uterine cancer, uterine leiomyomas, ovarian cancer, endometrial cancer, polycystic ovary syndrome, endometrial polyps, squamous cell carcinoma, squamous cell carcinoma of the head and neck, hepatocellular carcinoma, intrahepatic metastasis of hepatocellular carcinoma, prostate cancer, prostatic hypertrophy, pituitary cancer, adenomyosis, adenocarcinoma, pancreatic adenocarcinoma, meningioma, melanoma, bone cancer, multiple myeloma, CNS cancer, glioma, or astroblastoma.

43. A compound of Formula II: 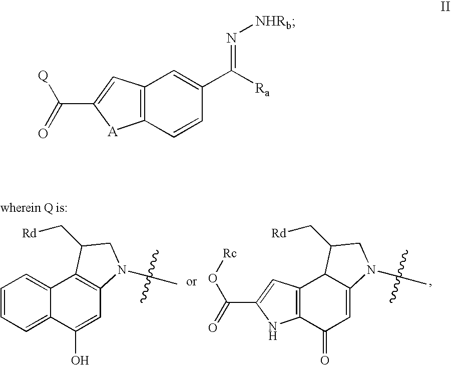
A is NH, O or S;
Rₐ is H or alkyl;
R_{b} is H, alkyl or —C(═O)—(CH₂)ᵣ—N-maleimide;
R_{c} is alkyl;
R_{d} is halogen; and
r is 2, 3, 4, 5 or 6;
or a stereoisomer, prodrug, pharmaceutically acceptable salt, hydrate, solvate, acid salt hydrate, N-oxide or isomorphic crystalline form thereof.

44. A compound 1-[3-(N′-{1-[2-(1-Chloromethyl-5-hydroxy-1,2-dihydro-3H-benzo[e]indole-3-carbonyl)-1H-indol-5-yl]-ethylidene}-hydrazino)-3-oxo-1-propyl]maleimide.

45. A pharmaceutical composition comprising at least one pharmaceutically acceptable carrier or excipient and an effective amount of the compound of claim 43, wherein the maleimide moiety is conjugated to a single chain Fv antibody.

46. The composition of claim 45 wherein said single chain Fv antibody is an antibody to an integrin receptor.

47. The composition of claim 46 wherein said integrin receptor is an-α₃β₁ integrin receptor or an αvβ3 integrin receptor.

48. A method comprising administering to a mammal the composition of claim 46.

49. A method for alleviating a disease state in a mammal believed to be responsive to treatment with an antibody conjugated to a carbonyl-substituted CBI-indole analog of duocarmycin SA comprising the step of administering to the mammal a therapeutic amount of the composition of claim 46.

50. The method of claim 49 wherein said disease state is neoplastic disease.

51. The method of claim 50 wherein the neoplastic disease is selected from solid tumor, hematological malignancy, leukemia, colorectal cancer, benign or malignant breast cancer, uterine cancer, uterine leiomyomas, ovarian cancer, endometrial cancer, polycystic ovary syndrome, endometrial polyps, squamous cell carcinoma, squamous cell carcinoma of the head and neck, hepatocellular carcinoma, intrahepatic metastasis of hepatocellular carcinoma, prostate cancer, prostatic hypertrophy, pituitary cancer, adenomyosis, adenocarcinoma, pancreatic adenocarcinoma, meningioma, melanoma, bone cancer, multiple myeloma, CNS cancer, glioma, or astroblastoma.
